(19) United States
(12) Patent Application Publication
(10) Pub. No.: US 2010/0173364 A1
(43) Pub. Date: Jul. 8, 2010

(54) Repair of Nucleic Acids for Improved Amplification

(75) Inventors: Thomas C. Evans, JR., Topsfield, MA (US); Barton Slatko, Ipswich, MA (US); Lixin Chen, Beverly, MA (US); Romaldas Vaisvila, Ipswich, MA (US); Chudi Guan, Wenham, MA (US); Rebecca Kucera, Hamilton, MA (US)

Correspondence Address: HARRIET M. STRIMPEL, D. Phil., New England Biolabs, Inc., 240 COUNTY ROAD, IPSWICH, MA, 01938-2723, US

(73) Assignee: NEW ENGLAND BIOLABS, INC., Ipswich, MA (US)

(21) Appl. No.: 12/296,827

(22) PCT Filed: Apr. 11, 2007
(86) PCT No.: PCT/US07/08792

§ 371 Date: Mar. 24, 2010

Related U.S. Application Data
(60) Provisional application No. 60/791,056, filed on Apr. 11, 2006.

(51) Int. Cl. C12P 19/34; C12N 9/00; C12N 9/10

(52) U.S. Cl. 435/91.2; 435/91.52; 435/183; 435/193

(57) ABSTRACT

Methods and compositions are provided for repairing a polynucleotide so that it can be copied with improved fidelity and/o yield in, for example, an amplification reaction. This involves the use of a reaction mixture that includes a DNA ligase and an effective amount of at least one endonuclease as well as a cofactor selected from NAD⁺ or ATP.

### BACKGROUND

Copying of polynucleotides, more particularly amplification, is commonly used in molecular biology for studying, for example, the properties of genes. Problems in copying arise when the polynucleotide is damaged in some way.

By way of illustration, U.S. Pat. No. 5,035,996 describes a process for controlling contamination of polymerase chain reaction (PCR) amplification reactions that uses the modified nucleotide, dUTP, in the amplification reaction. This process uses uracil DNA glycosylase (UDG) to eliminate those PCR products containing uracil to prevent contaminating subsequent PCR reactions. U.S. patent publication No. 2004-0067559 A1 also relies on modified bases in primer DNA prior to amplification and uses, for example, dUTP for incorporation into the amplicon. The amplicon can then be fragmented by adding, for example, UDG and endonuclease IV.

One amplification methodology referred to as hot start nucleic acid amplification has been used to lower mis-priming during PCR. In one type of hot start amplification, prevention of extension by the polymerase relies on the presence of a PCR primer with a blocked 3′ terminus in the PCR reaction (see for example U.S. Publication No. 2003-0119150). The primer is unblocked by a thermostable 3′-5′ exonuclease that is active at a temperature of greater than 37° C. Therefore, the DNA polymerase will only extend the PCR primers once the exonuclease unblocks the 3′ end at temperatures greater than 37° C. Alternatively the Thermus aquaticus (Taq) polymerase is blocked and then activated at amplification temperatures.

Barnes, W. M. Proc. Natl. Acad. Sci. USA 91:2216-2220 (1994) describes the use of Vent® polymerase and Taq polymerase as an improvement over the use of Taq polymerase only in amplification. Ghadessy et al. reported a mutant Taq polymerase that is not halted by damaged or abasic sites (Ghadessy et al. Nature Biotechnol. 22(6):755-9 (2004)).

It has been reported that conventional amplification techniques are compromised if the DNA is substantially damaged (Di Bernardo et al. Nucl. Acids Res. 30:e16 (2002)). Degradation and/or fragmentation of DNA resulting from exposure to the environment and microorganisms which contain DNA nucleases is a frequent problem in forensics, diagnostic tests and routine amplification and affects fidelity and yield of the amplification product. In addition, the problem of degraded DNA is also faced by researchers who are analyzing the DNA obtained from extinct or preserved organisms.

Fromenty, B., et al. Nucl. Acids Res. 28(11):e50 (2000) and International Publication No. WO 01/051656 reported that treatment with exonuclease III improved yields of long PCR. However, Fromenty also reported decreased yields of amplicon for DNA less than 500 by when exonuclease III was used. One of the problems associated with the use of exonuclease III at the concentrations described is that it degrades template and primers. The use of exonuclease III for DNA repair is also described by Walker et al. in U.S. Publication No. 05/0026147 in an enzyme blend that also consists of AccuTaq LA DNA polymerase and DTT (Example 2 of the reference). The reference describes a requirement for an additional heat inactivation step to inactivate the exonuclease III prior to adding oligonucleotide primers. Preparations are sold commercially by Sigma, St. Louis, Mo. and Qbiogene, now MP Biomedicals, Irvine, Calif. for use with DNA prior to DNA amplification although the content of these preparations is not specified by the supplier. A limitation of the approach described in these references is a reported need for a denaturation step after repair and prior to amplification. It would be desirable to accomplish repair in a single step and to merely add reagents to accomplish amplification without an additional separation or denaturation step.

Di Benardo et al. Nucl. Acids Res. 30(4):e16 (2002) described the use of T4 DNA ligase (T4 DNA ligase) and an E. coli polymerase as a pretreatment to amplify short regions of single-stranded DNA between cross-linked regions of double-stranded DNA.

Another approach to amplification of damaged DNA has been described in U.S. Publication No. 2003-0077581. Degraded nucleic acid was hybridized to non-degraded nucleic acid having a sequence homologous to the degraded nucleic acid. Regions of the degraded nucleic acid were then filled in with nucleotide precursors. The fragmented strands were then covalently linked using a polymerizing and/or ligating enzyme.

Others report the use of a combination of E. coli DNA pol I and T4 DNA ligase for pre-amplification repair (Pusch, et al., Nucl. Acids Res. 26:857 (1998)). However, according to Pusch et al., the pre-amplification product must be purified before initiation of amplification. Eschoo (US publication 2006/0014154) also describes the need for a purification step prior to amplification.

### SUMMARY

In an embodiment of the invention, a method is provided for enhancing at least one of fidelity and yield of a copied or amplified product by repairing a damaged polynucleotide such as but not limited to DNA. The method includes incubating the polynucleotide in a reaction mixture comprising an effective amount of at least one AP endonuclease, a DNA ligase and at least one of NAD⁺ or ATP as a cofactor. An NAD⁺-dependent DNA ligase is selected for certain uses of the method such as PCR amplification or whole genome amplification. Where ATP is utilized, a concentration of less than 500 μM ATP may be used that minimizes the negative effect on subsequent amplification of DNA.

Repair of the polynucleotide in the reaction mixture may be accomplished at a single temperature (within the limits of temperature fluctuations of a standard incubator) prior to amplification or copying. For example, the isothermal temperature may be selected from the range of 4° C. to 52° C. for an incubation time in the range of 1 minute to 12 hours.

In the embodiments of the invention described herein, a temperature or other denaturation step during or after repair and prior to copying or amplification is not required. Nor is a purification step required between repair and copying or amplification. Repair and amplification or copying can therefore be achieved in a single step.

Amplification can be achieved by PCR amplification, helicase-dependent amplification, strand-displacement amplification, rolling circle amplification, whole genome amplification or other amplification protocol known in the art.

In embodiments of the method, the polynucleotide is obtained from a source selected from the group consisting of: a natural source, preserved biological material, forensic evidence, ancient material of biological origin, a tissue biopsy and chemical synthesis. The type of damage to the polynucleotide include: apurinic/apyrimidinic (AP) sites, mutagenized nucleotides, modified nucleotides, nicks, gaps, DNA-DNA or DNA-protein cross-links, and DNA-RNA crosslinks.

The DNA ligase in the reaction mixture may be a thermostable ligase. For example, an ATP-dependent ligase such as 9° N ligase or an NAD⁺-dependent ligase such as Taq DNA ligase may be used. Alternatively, a mesophilic ligase may be used such as E. coli DNA ligase where the required cofactor is NAD⁺.

The one or more AP endonucleases with an effective amount of specific AP endonuclease activity may be obtained from a bacterium such as E. coli, a mammal such as human, an archaea such as Thermococcus, or a virus such as African swine fever virus.

The reaction mixture may further include a Family A, B or Y DNA polymerase such as a Taq DNA polymerase, an E. coli DNA polymerase, a Bst DNA polymerase, a phage T4 DNA polymerase or a phage T7 DNA polymerase, E. coli pol IV, E. coli pol V, human pol kappa, human pol eta, Sso Dpo4, Sac Dbh, Sce pol zeta and human pol iota.

In an additional embodiment, a reaction mixture is provided that further includes T4 pyrimidine dimer glycosylase (PDG) and/or formamidopyrimidine [fapy]-DNA glycosylase (Fpg), and/or at least one of UvrA, UvrB, and UvrC and optionally UvrD or Cho. Optionally, the reaction mixture may further include T7 endonuclease I or a mutant thereof, such as described in U.S. publication number 2007/0042379.

In an additional embodiment, the reaction mixture may further include at least one of endonuclease VIII, endonuclease V or endonuclease III, UDG and alkyl adenine DNA glycosylase (Aag).

In an embodiment of the invention, a kit is provided that includes: two or more enzymes wherein at least one of the enzymes is a DNA ligase and at least one of the enzymes is an AP endonuclease having a concentration of 0.0001 units/μl to 100 units/μl of reaction mixture, the two or more enzymes being formulated for addition to a damaged polynucleotide preparation to enhance repair of the polynucleotide; and instructions for its use.

In another embodiment of the invention, a polynucleotide repair mixture is provided that includes a DNA ligase, a DNA polymerase, and an effective amount of at least one AP endonuclease, in a buffer suitable for (1) addition to an amplification mix; and (2) permitting enhancement of at least one of yield and fidelity of a copied or amplified polynucleotide compared with a copied or amplified polynucleotide in the absence of the polynucleotide repair mixture.

The DNA polymerase may be a Bst DNA polymerase. The polynucleotide repair mixture may additionally include a T4 PDG. In addition, the polynucleotide repair mixture may include an E. coli Fpg. The polynucleotide repair mixture may further include at least one of UvrA, UvrB, UvrC and optionally UvrD or Cho. UvrA, UvrB, UvrC, UvrD and Cho may be obtained from bacteria such as E. coli, or eukaryotic equivalents may be used. The polynucleotide repair mixture may further include at least one of endonuclease VIII, endonuclease V or endonuclease III. The polynucleotide repair mixture may further include at least one of UDG and Aag. The composition may further include a PDG, a UDG, an endonuclease VIII and/or an Fpg.

In embodiments of the invention, the polynucleotide repair mixture includes one or more of the DNA ligase, DNA polymerase, AP endonuclease, PDG, UDG, endonuclease VIII and Fpg obtained from E. coli. For example, the AP endonuclease, endonuclease VIII, UDG, and Fpg in the polynucleotide repair mixture can all be obtained from E. coli. In these embodiments, the PDG can be T4 PDG, the DNA ligase can be Taq DNA ligase, and the DNA polymerase can be Bst DNA polymerase.

In embodiments of the invention, the enzyme concentration in the polynucleotide repair mixture is in the range described below: T4 PDG in a concentration range of 0.0001 units/μl to 4 units/μl. Taq DNA ligase in a concentration range of 0.00001 units/μl to 100 units/μl. Bst DNA polymerase in a concentration range of 0.00001 units/μl to 2 units/μl, E. coli endonuclease IV in the range of 0.0001 units/μl to 100 units/μl, endonuclease VIII in the range of 0.00001 units/μl to 20 units/μl, UDG in the range of 0.00001 units/μl to 20 units/μl, and Fpg in the range of 0.000001 units/μl to 0.1 units/μl.

In an embodiment of the invention, a method for cloning or sequencing a polynucleotide fragment is provided that includes: repairing sequence errors in the polynucleotide fragment by means of a polynucleotide repair mixture described above and cloning or sequencing the polynucleotide fragment. The polynucleotide repair mixture may cause blunt ending of the polynucleotide for cloning into a vector.

In an embodiment of the invention, a method is provided for enhancing the yield of a copied or amplified polynucleotide, that includes: (a) obtaining at least a first pair and a second pair of primers wherein the second pair of primers is nested within the first set of primers when hybridized to the polynucleotide; (b) subjecting the polynucleotide to a repair mixture described above; (c) amplifying the polynucleotide with the first set of primers; (d) amplifying the product of (c) with the second set of primers; and (e) obtaining an enhanced yield of amplified polynucleotide.

In the method of cloning and the method for enhancing yield of a copied or amplified polynucleotide described above, the composition may contain a DNA ligase such as Taq DNA ligase, a DNA polymerase such as Bst DNA polymerase, a PDG such as T4 PDG, and an endonuclease IV, an endonuclease VIII, Fpg and optionally UDG such as those derived from E. coli.

In an embodiment of the invention, a method is provided for sequencing a polynucleotide, including: (a) contacting the polynucleotide with a composition that includes an effective amount of a DNA ligase, a DNA polymerase and a concentration of an AP endonuclease lacking substantial non-specific DNA degradative activity in a buffer that is compatible with a sequencing reaction; and (b) sequencing the polynucleotide.

In another embodiment of the invention, a method for copying or amplifying a fragmented DNA, is provided that includes: (a) contacting the fragmented DNA with a composition that contains an effective amount of a DNA ligase, a DNA polymerase and a concentration of an AP endonuclease lacking substantial exonuclease activity in a buffer that is compatible with an amplifying or copying reaction; (b) optionally adding a recombination-competent protein such as E. coli recA or phage lambda beta protein; and (c) amplifying or copying the fragmented DNA.

### BRIEF DESCRIPTION OF THE DRAWINGS

FIGS. 1A-1D show enhanced amplicon yield from heat-damaged lambda DNA after pre-incubation with specified enzymes.

FIG. 1A shows DNA template damaged to differing extents by heat and the effect of this damage on amplification of a 5 kb segment of lambda DNA where 5 ng, 2 ng and 1 ng of heat-treated lambda DNA were amplified after prior damage by 99° C. heat treatment for 0 sec, 30 sec, 60 sec, 90 sec, 120 sec or 180 sec. The damaged DNA was not subjected to enzyme treatment prior to amplification. The amount of amplification was determined after electrophoresis and was found to be substantially reduced by 120 sec heat treatment. The first lane on the gel contains 1 μg of a 2-log ladder size standard ((NEB#N3200, New England Biolabs, Inc., (NEB), Ipswich, Mass.)).

FIG. 1B shows enhanced amplicon yields from heat-damaged lambda DNA using Taq DNA ligase, E. coli endonuclease IV and E. coli pol I on amplification of a 5 kb segment of lambda DNA. DNA was heat-damaged as described in FIG. 1A but the damaged DNA was subjected to enzyme treatment prior to amplification. The results of amplification are shown after a 10-minute pretreatment reaction with Taq DNA ligase, E. coli endonuclease IV and E. coli pol I. The amplicon yield was increased throughout but was especially noticeable with 120 sec and 180 sec heat-damaged DNA. First and last lanes on the gel contain 1 μg of a 2-log ladder size standard (NEB#N3200, NEB, Ipswich, Mass.).

FIG. 1C shows enhanced amplicon yields from heat-damaged lambda DNA using Taq DNA ligase, Thermus thermophilus (Tth) endonuclease IV and E. coli pol I. The amplification was performed according to FIG. 1B but the enzyme treatment prior to amplification contained Tth endonuclease IV in place of E. coli endonuclease IV. The results of amplification are shown after a 10-minute pretreatment reaction with Taq DNA ligase, Tth endonuclease IV and E. coli pol I. The amplicon yield was increased throughout but was especially noticeable with 120 sec and 180 sec heat-damaged DNA. Only the first lane contains the molecular weight marker ladder.

FIG. 1D shows enhanced amplicon yields from heat-damaged lambda DNA using E. coli DNA ligase, E. coli endonuclease IV and E. coli DNA pol I. The amplification was performed according to FIG. 1B but the enzyme treatment prior to amplification contained E. coli DNA ligase in place of Taq DNA ligase. The lambda DNA subjected to 99° C. for 180 sec was used as a template. The amount of template DNA used is indicated above each lane. The yield of 5 kb amplicon is enhanced for each of the template amounts by enzyme pretreatment.

FIGS. 2A and 2B show the effect of citrate buffer pH 5 treatment of template DNA on amplicon yield.

FIG. 2A shows the results of amplification of a 5 kb segment of lambda DNA where lambda DNA was heated to 70° C. in citrate buffer pH 5 for 0, 20, 40, 80, 120, and 160 minutes. 50 ng, 10 ng and 5 ng of each citrate treated sample were amplified and the resulting products were visualized on a gel to determine the extent of amplification. The DNA was not treated with selected enzymes prior to amplification. The last lane on the right contains 1 μg of 2-log ladder.

FIG. 2B shows the increase in yield of a 5 kb amplicon of lambda DNA regardless of which DNA polymerase was used in the enzyme mixture. 120-minute citrate-damaged lambda DNA was treated with various enzymes prior to amplification.

Lane 1: 1 μg 2-log ladder (NEB# N3200, NEB, Ipswich, Mass.).

Lane 2: no pretreatment.

Lane 3: Pretreatment with Taq DNA ligase, Taq DNA polymerase and E. coli endonuclease IV.

Lane 4: Pretreatment with Taq DNA ligase, E. coli pol I, and E. coli endonuclease IV.

Lane 5: Pretreatment with Taq DNA ligase, Taq:Vent® DNA polymerase mix, and E. coli endonuclease IV.

FIG. 3 shows the results of amplification of a 200 bp segment of krill genome that has been extracted from an ethanol stored sample of krill and pretreated with an enzyme mixture containing one of various DNA polymerases, a DNA ligase and an AP endonuclease that enhances amplification yields.

Lane 1: No pretreatment of krill DNA with enzymes.

Lane 2: Pretreatment of krill DNA with Taq DNA ligase, E. coli endonuclease IV, and Taq DNA polymerase.

Lane 3: Pretreatment of krill DNA with Taq DNA ligase, E. coli endonuclease IV, and Vent® polymerase.

Lane 4: Pretreatment of krill DNA with Taq DNA ligase, E. coli endonuclease IV, and 50:1 Taq:Vent® DNA polymerase.

FIG. 4 shows an increase in yield of a 10 kb amplicon from heat-damaged DNA. 180 sec heat-damaged DNA was pretreated with an enzyme mixture and then amplified.

Lane 1: 1 μg of a 2-log ladder size standard (NEB#N3200, NEB, Ipswich, Mass.).

Lane 2: Pre-treatment with Taq DNA ligase, E. coli endonuclease IV, and E. coli pol I.

Lane 3: Pre-treatment with Taq DNA ligase and E. coli endonuclease IV.

Lane 4: Pretreatment with Taq DNA ligase.

Lane 5: Control—untreated DNA.

FIG. 5 shows that DNA ligase pretreatment increases amplicon yield from environmental DNA (soil sample extract).

Lane 1: A 2-log ladder size standard (NEB# N3200, NEB, Ipswich, Mass.).

Lane 1: No enzyme pretreatment.

Lane 2: Pre-treatment with T4 DNA ligase.

Lane 3: No enzyme pre-treatment.

Lane 4: Pretreatment with Taq DNA ligase.

FIGS. 6A-1-6A-9 and 6B-1-6B-2: Blast P search at NCBI using E. coli DNA ligase (A) and T4 DNA ligase (B).

FIG. 7 shows the DNA sequence of Tth endonuclease IV (SEQ ID NO:11).

FIGS. 8A, 8B and 8C show the effect of UV light on amplicon yield using lambda DNA.

FIG. 8A: Lambda DNA is subjected to UV-irradiation for up to 50 sec and a slight reduction in yield of a 2 kb amplicon produced is shown.

FIG. 8B: Lambda DNA is subjected to UV-irradiation for up to 50 seconds and the reduction in yield of a 5 kb amplicon is shown.

FIG. 8C: The effect of various reaction mixtures added to lambda DNA on yield of a 5 kb amplicon after UV-irradiation is shown.

Lanes 2-7 are controls in the absence of a reaction mixture.

Lanes 8-13 show the increased beneficial effect of adding ligase, DNA polymerase and AP endonuclease plus 10 units of T4 pdg.

Lanes 14-19 show the increased beneficial effect of adding DNA ligase, DNA polymerase and AP endonuclease plus 80 units of T4 pdg.

Lanes 1 and 20: A 2-log ladder size standard (NEB#N3200, NEB, Ipswich, Mass.).

FIGS. 9A and 9B show that adding DNA ligase to T7 endonuclease I expands the useful range of the EndoI:DNA ratio in which the product is not degraded. Taq DNA ligase and T7 endonuclease I were added to supercoiled DNA in varying amounts as indicated for each lane.

FIG. 9A is the control in which no Taq DNA ligase has been added but increasing amounts of T7 endonuclease I were used. The supercoiled DNA is predominantly cleaved into fragments of various sizes with 12.5-25 units of T7 endonuclease I.

FIG. 9B shows how the addition of 100 units of Taq DNA ligase protects DNA from non-specific cleavage in the presence of T7 endonuclease I such that even at 200 units of T7 endonuclease I, there is a clear band corresponding to linear DNA not present in the absence of DNA ligase.

FIGS. 10A and 10B show the effect of repair enzyme treatment on amplicon yield from oxidatively damaged DNA or undamaged template.

FIG. 10A shows that the addition of repair enzymes to an undamaged template, pWB407 has no effect on amplicon yield.

FIG. 10B shows that the addition of Fpg to a damaged template, plasmid pWB407, which was previously incubated in the presence of methylene blue, gives inconsistent effects on yield. The addition of Taq DNA ligase, E. coli DNA polymerase, and E. coli endonuclease IV in the presence or absence of Fpg consistently increases amplicon yield.

FIG. 11 shows increased PCR reaction fidelity from damaged DNA after treatment with repair enzymes. Repair enzyme treatment of undamaged template, plasmid pWB407, prior to PCR has no significant effect on fidelity. Treatment of a damaged template, plasmid pWB407 incubated with methylene blue, with Fpg alone or also with Taq DNA ligase, E. coli DNA polymerase I, and E. coli endonuclease increases the fidelity of PCR. The measure of fidelity is the number of white colonies verses the number of blue colonies after cloning a lacZ-containing amplicon as discussed below. The higher the percentage of white colonies the greater the error rate.

FIG. 12 shows a flow diagram for treating damaged DNA or to increase at least of one of fidelity or yield.

FIGS. 13A and 13B show how yield of amplicon is increased for a 5 kb fragment of 30s UV-damaged DNA incubated for 15 minutes at room temperature or at 4° C. overnight with a multi-enzyme repair mix.

FIG. 13A: Room temperature incubation:

Lane 1: 2-log ladder DNA molecular weight standard.

Lanes 2 and 3: the two reactions incubated without the multi-enzyme repair mix at room temperature for 15 minutes.

Lanes 4 and 5: the reactions incubated with the repair mix at room temperature for 15 minutes have the expected 5 kb amplicon.

FIG. 13B: 4° C. incubation:

Lane 1: 2-log ladder DNA molecular weight standard.

Lanes 2 and 3: the two reactions incubated without the multi-enzyme repair mix overnight at 4° C.

Lanes 4 and 5: the reactions incubated with the repair mix overnight at 4° C. have the expected 5 kb amplicon.

FIG. 14 shows enhanced amplicon yield from a uracil-containing plasmid after treatment with a repair enzyme mix for 15 mins at room temperature and PCR amplification using an archaeal DNA polymerase. Lanes 1 and 2 shows the product of PCR amplification of pNEB0.92U using Vent® DNA polymerase. There is a weakly visible band at 920 bp. Lanes 3 and 4 show the product of PCR amplification from pNEB0.92U treated with a repair enzyme mix.

FIG. 15 shows an agarose gel on which a band corresponding to an amplified DNA of 620 base pairs is identified. The 620 by amplicon was obtained from 20 overlapping single strand oligonucleotides of 48 nucleotides or smaller.

Lane 1: 2-log DNA molecular weight standards (NEB#N3200S, NEB, Ipswich, Mass.).

Lane 2: 20 oligonucleotides incubated with 400 units Taq DNA ligase, 0.1 units E. coli pol I, 5 units T4 pdg, and 20 units endonuclease IV during the assembly step.

Lane 3: 20 oligonucleotides incubated with 400 units Taq DNA ligase, 0.1 units E. coli pol I, 5 units T4 PDG, 20 units endonuclease IV, and lambda beta protein during the assembly step.

Lane 4: 20 oligonucleotides incubated with 400 units Taq DNA ligase, 0.1 units E. coli pol I, 5 units T4 PDG, 20 units endonuclease IV, and E. coli RecA during the assembly step.

Lane 5: 20 oligonucleotides incubated with 400 units Taq DNA ligase, 0.1 units E. coli pol I, 5 units T4 PDG, 20 units endonuclease IV, lambda beta protein and RecA during the assembly step.

Lane 6: the control, 20 oligonucleotides with no added repair enzymes during the assembly step.

FIG. 16 shows the effect of DNA repair treatment on non-irradiated and irradiated DNA as determined by the number of colonies obtained when the DNA containing a selection marker is used to transform cells.

FIG. 17 shows improved yields of amplicon from ancient cave bear DNA after 2 sets of amplification reactions using different nested primer pairs. The gene map shows the location of primer pairs F1-R1, F1-R2 and F1-R4. Above the gels, a set of numbers is provided (88, 79, 10, 11, 1868 and 1314) that represent the estimated amount of mitochondrial DNA in each sample. Lanes 3A and 3B contain the most DNA. +/− indicates whether a repair mix was used prior to the first amplification using F1-R1. In Lane 3B, a sharp band corresponding to repaired cave bear DNA was observed that was not present in the absence of repair.

FIG. 18 shows the DNA sequence for plasmid pNEB0.92U (SEQ ID NO:42).

FIG. 19 shows the amplification products resulting from repaired DNA compared with controls.

Lanes 1 and 2 show control DNA and UV-damaged lambda DNA

Lanes 3 and 4 show control DNA and heat-damaged lambda DNA

Lanes 5 and 6 show control DNA and oxidized plasmid DNA

Lanes 7 and 8 show control DNA and UV-damaged human genomic DNA

Lanes 9 and 10 show control DNA and UV- and AP-damaged lambda DNA

Lanes 11 and 12 show control DNA and AP-damaged lambda DNA

FIGS. 20A-20B shows the effect of increasing concentrations of ATP on the ability to amplify a 5 kb amplicon from a lambda DNA template. The reaction was performed in triplicate at each of eight ATP concentrations tested. FIG. 20A shows the reactions that contained 0, 15, 30 and 60 μM ATP. FIG. 20B is a continuation of the titration and shows the effect of ATP at 120, 240, 480 and 960 μM. The presence of 960 μM ATP resulted in no detectable amplicon at an ATP concentration of 960 μM in the PCR reaction. The reactions were subjected to electrophoresis on a 1% agarose gel and visualized by ethidium bromide staining. The left-hand lanes in FIGS. 20A and 20B are a broad range molecular weight DNA marker.

### DETAILED DESCRIPTION OF EMBODIMENTS

Embodiments of the methods, have wide utility in molecular biology research and in solving problems in applied biology including, for example, analyzing fragmented and damaged DNA such as found in forensic analysis, in biological archeology in which it is desirable to analyze DNA from ancient sources, for taxonomy where it is desirable to analyze DNA from environmental samples such as required for the Barcode of Life Project, and for diagnostic assays including tissue biopsies to determine a disease susceptibility or status. Other uses include: high-fidelity sequencing, gene assembly, fragment analysis and copying, ligation for cloning and one-step repair and blunt-ending.

Most polynucleotides that are isolated or in vitro-replicated are damaged to some extent. Damage of a polynucleotide may result from chemical modification of individual nucleotides or disruption of the polynucleotide backbone. Polynucleotides experience damage from diverse sources such as chemicals including formaldehyde and methyl methanesulfonate, environmental factors, temperature extremes, oxidation, dessication and ultra-violet (UV) light. Various types of damage include: (a) apurinic or apyrimidinic damage caused for example by heat, and exposure to factors in the environment such as H₂O or extremes of pH; (b) modification of individual nucleotides caused for example by deamination, alkylation, and oxidation; (c) nicks and gaps caused for example by heat, and exposure to factors in the environment such as H₂O or extremes of pH; (d) cross-linking caused for example by formaldehyde, light or environmental factors; (e) mismatched DNA caused by for example misincorporation of a nucleotide by a DNA polymerase; and (f) fragmentation of DNA.

Damage is more severe in preserved tissues, dried specimens or polynucleotides that are exposed to the environment. Damage can occur as a result of the storage of the sample or its source or preparation. In addition, damage can occur during the application of a methodology for polynucleotide synthesis such as occurs during PCR amplification, which involves a high temperature step. Hence, most polynucleotides are damaged to some extent. This damage has a greater influence when longer amplicons are analyzed since the likelihood of encountering damage during amplification is increased.

Polynucleotides can sustain damage in a variety of ways. Different polynucleotide preparations experience different types of damage depending upon, for example, the storage or handling of the polynucleotide preparation in vitro, how prokaryotic cells, archaeal or eukaryotic cells containing the polynucleotides are stored and the characteristics of the cells from which the polynucleotides are extracted. Synthetic polynucleotides can sustain damage during chemical synthesis.

Embodiments of the invention provide improvements in the method for copying or amplifying damaged polynucleotides. These improvements can be achieved when the damaged polynucleotide is mixed with a reaction mixture before and/or during the copying or amplification step to enhance yield and for fidelity of the copied or amplified DNA. This is readily achieved by adding the polynucleotide to the reaction mixture containing a mixture of repair enzymes and cofactors. Enhanced yield and/or fidelity can improve the sensitivity and specificity of tests that rely on the characterization of the copied or amplified product. (“Enhanced” refers to obtaining an improved ratio of copied or amplified product to starting material with respect to yield and/or fidelity compared to the ratio observed in the absence of the repair mixture).

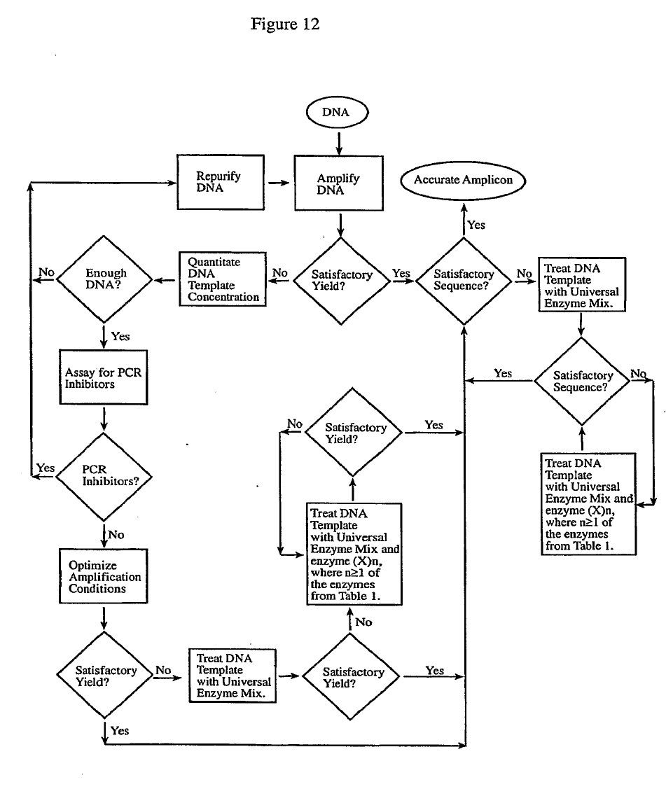

In an embodiment of the invention, the method provides for adding to a polynucleotide, a set of enzymes that can repair multiple different types of damage that commonly arise in the polynucleotides. This set of enzymes is here referred to as a universal mix. However, when a particular type of damage is targeted, a subset of the universal mix can be used providing that the subset minimally includes a DNA ligase, an AP endonuclease and a co-factor. In general, adding a plurality of enzymes to the polynucleotide in one step does not preclude adding one or more enzymes sequentially. FIG. 12 shows how an appropriate repair mixture may be selected according to whether the type of damage sustained by a polynucleotide is known or is unknown.

In various illustrative embodiments, the universal enzyme mixture contains Bst DNA polymerase, E. coli DNA polymerase I or Taq polymerase and an AP endonuclease such as a mesophilic endonuclease IV, e.g., E. coli endonuclease IV or a thermophilic endonuclease IV, e.g., Tth endonuclease IV and a DNA ligase selected from E. coli DNA ligase, Taq DNA ligase or an archaeal DNA ligase such as 9° N DNA ligase. The universal mix may further contain one or more of the following: T4 PDG, E. coli Fpg, at least one of UvrA, UvrB, UvrC and optionally UvrD or Cho, endonuclease VIII, endonuclease V, or endonuclease III, UDG and/or Aag.

The term “polynucleotide” refers in particular to double-stranded DNA, double-stranded RNA, hybrid DNA/RNA duplex, single-stranded DNA and single-stranded RNA.

A “repair enzyme” refers in particular to a psychrophilic, mesophilic or thermophilic enzyme that participates in the process of repair of a polynucleotide. For example, a repair enzyme may induce breakage of the polynucleotide at a bond, thereby facilitating removal of damaged regions of the polynucleotide or removal of single nucleotides. Enzymes with a synthetic role such as DNA ligases and DNA polymerases are also repair enzymes. However, in an embodiment of the invention, repair enzymes as used herein are not intended to include kinases. The damaged DNA is subjected to the reaction mixture so as to enhance copying and/or amplification of DNA. The repair reaction can be performed at a single temperature where a “single” temperature includes minor fluctuations in temperature that are associated with a water bath or refrigerator or other device used to set the temperature of the reaction.

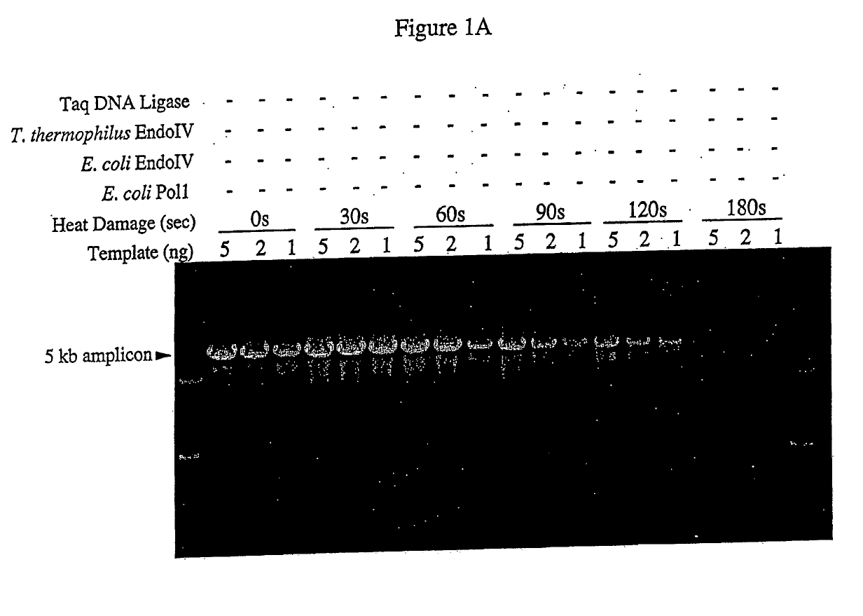

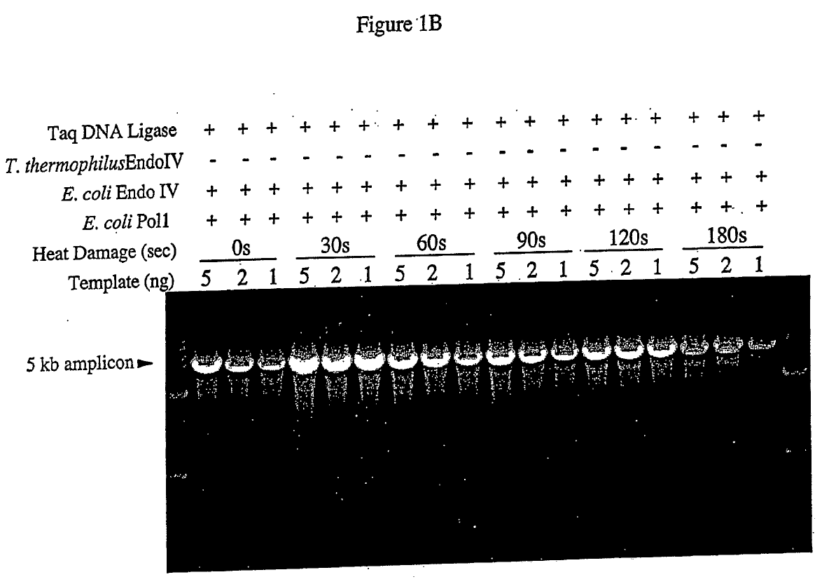

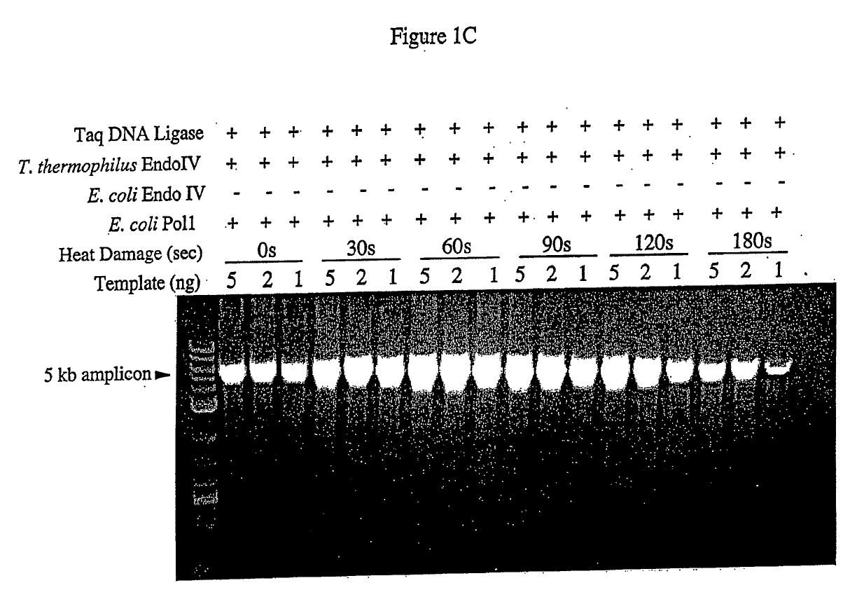

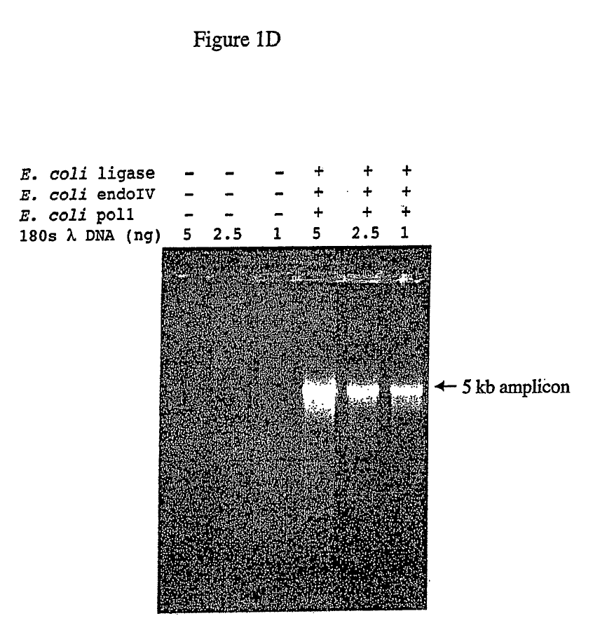

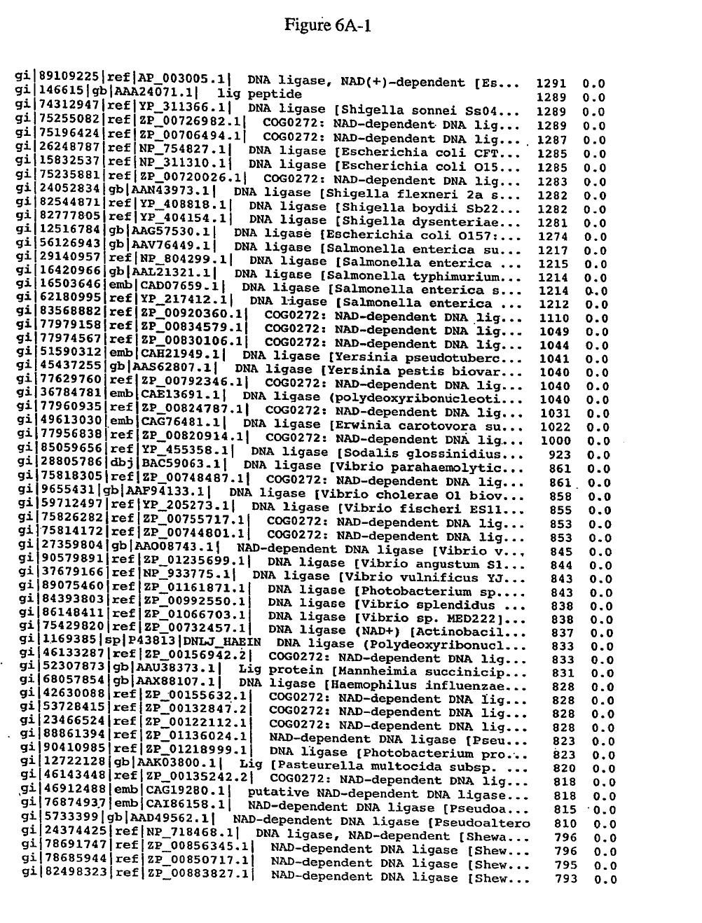

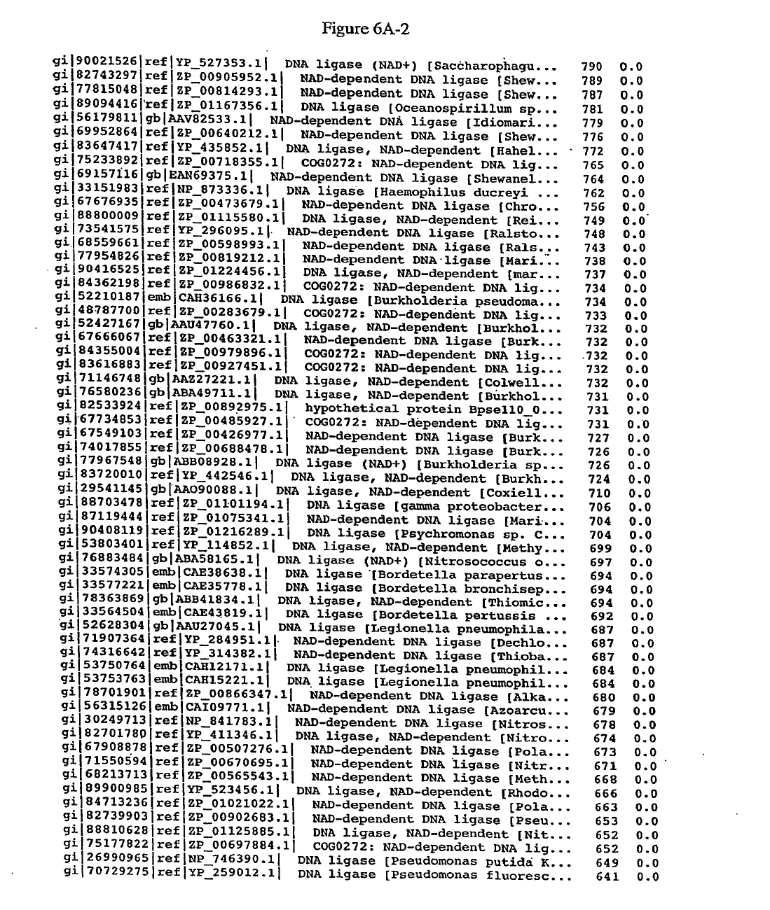

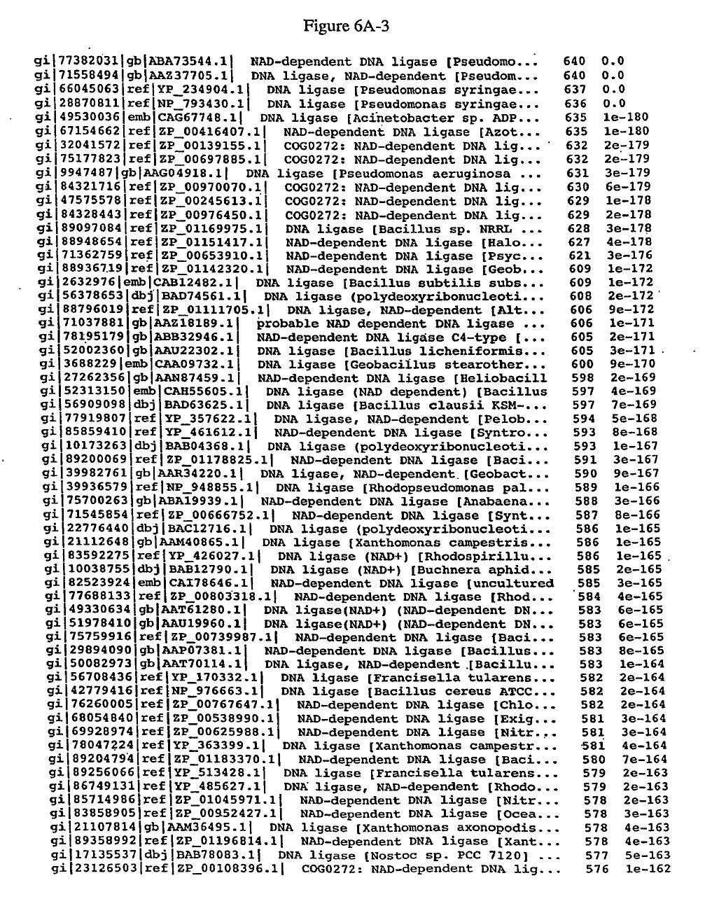

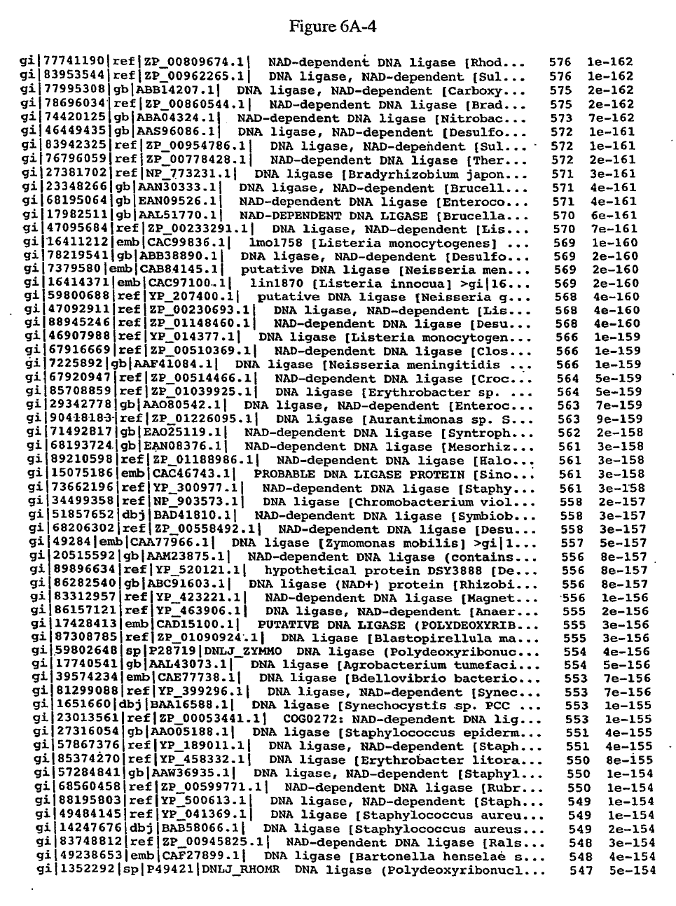

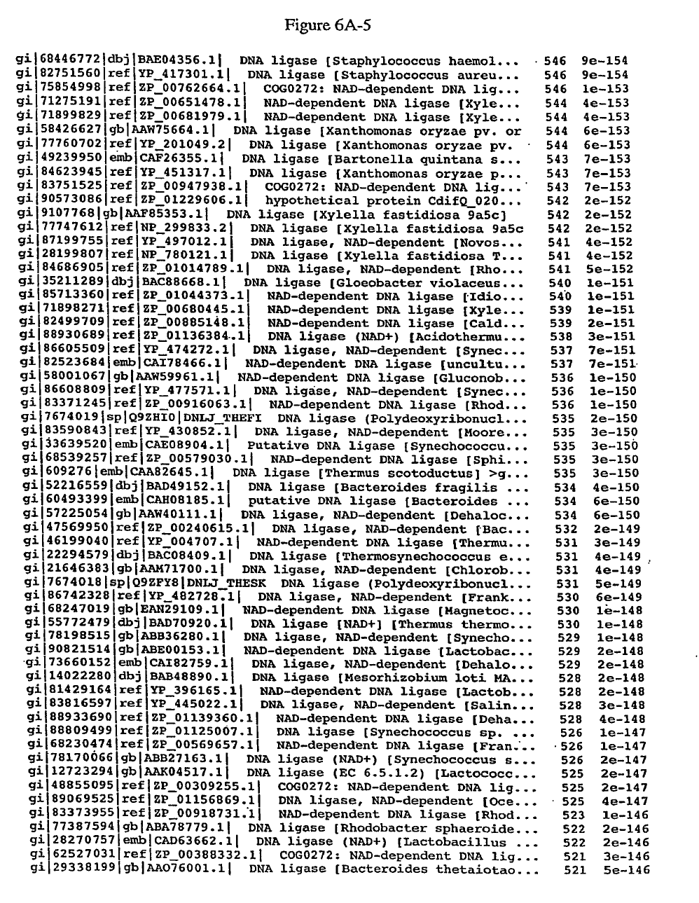

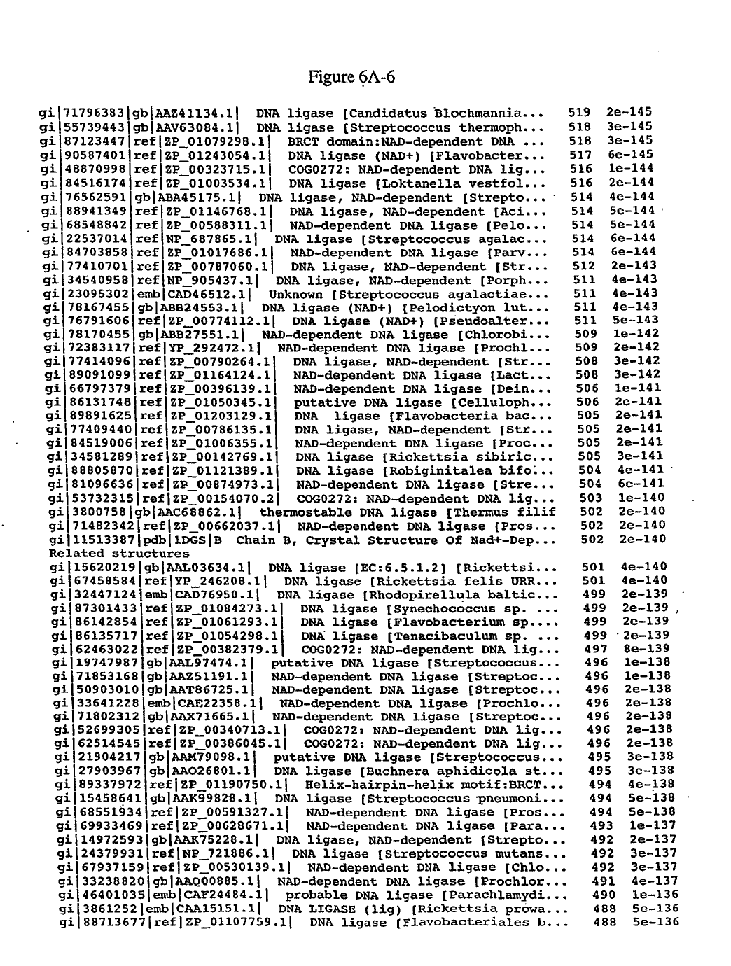

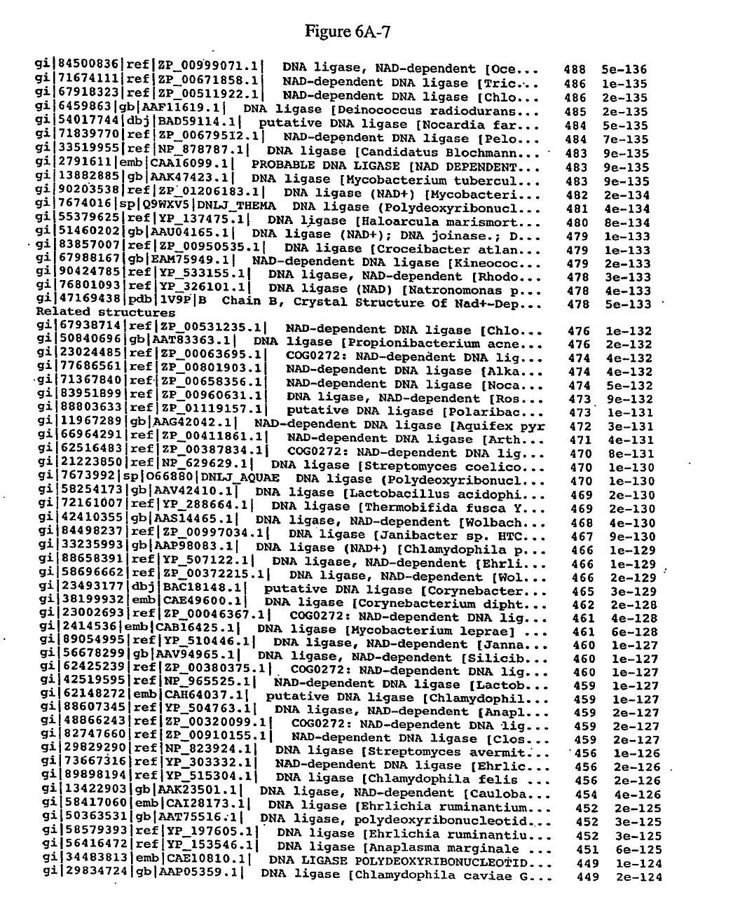

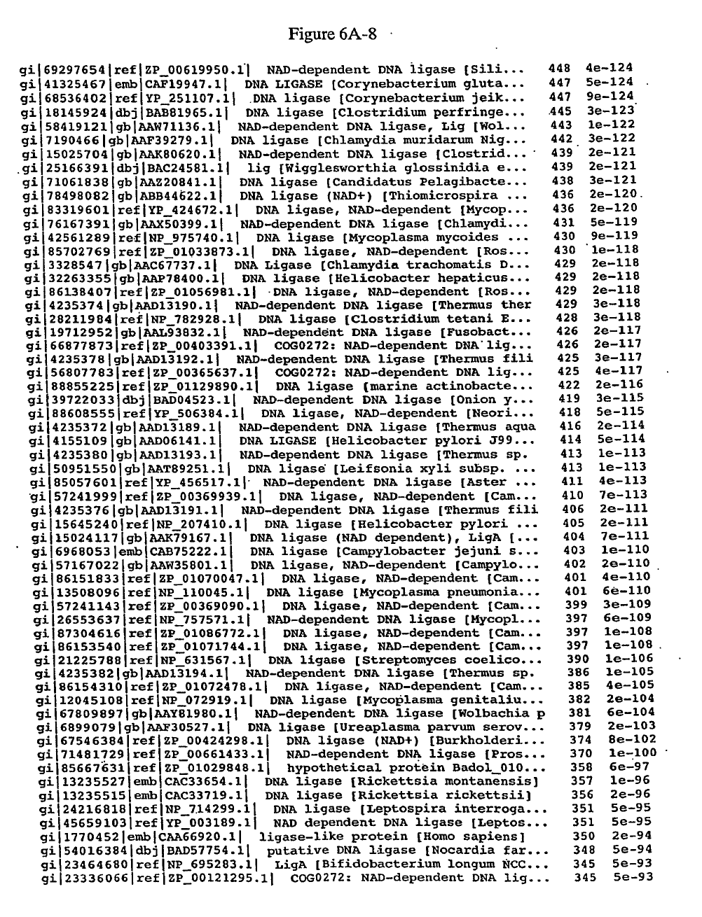

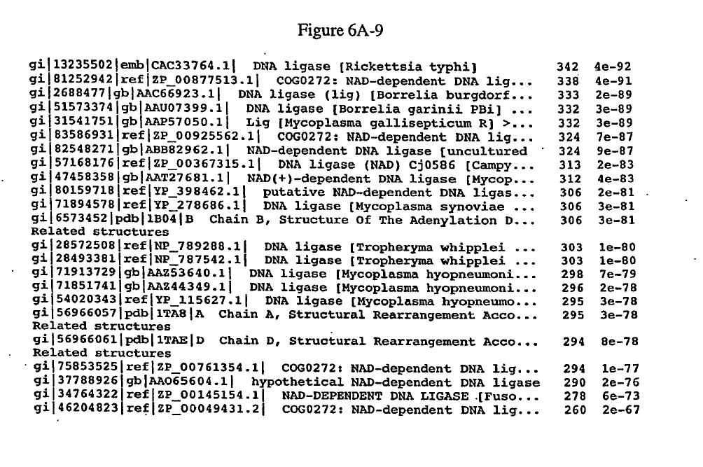

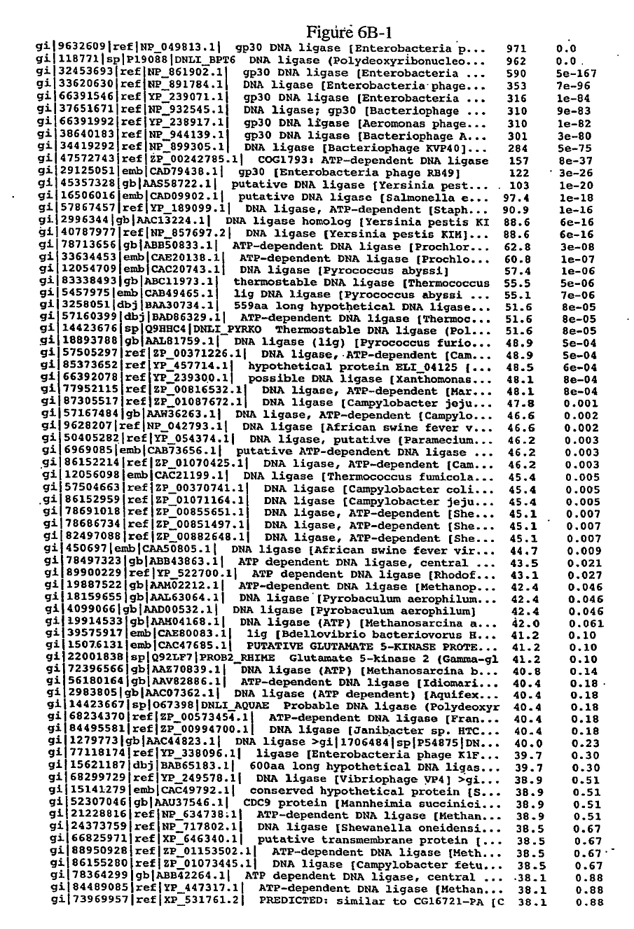

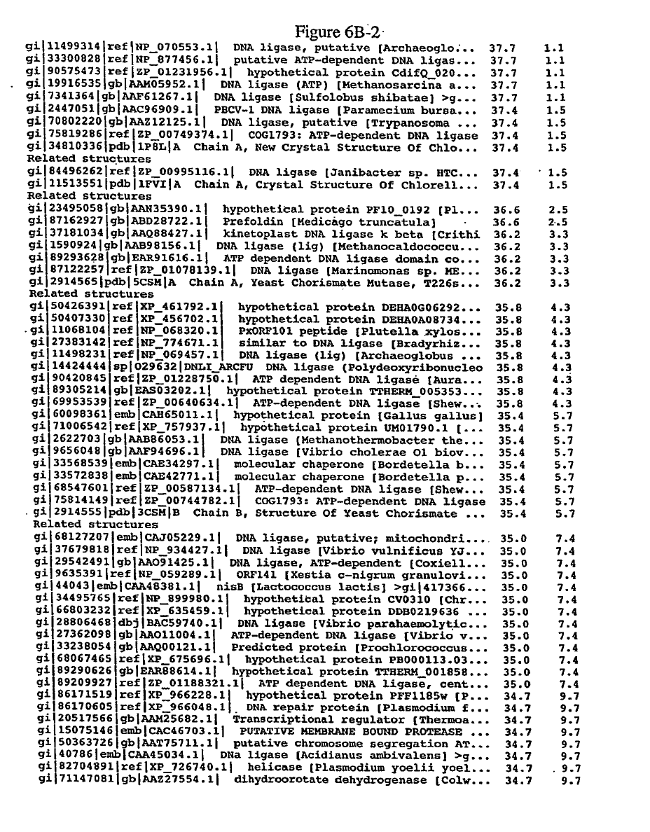

DNA repair enzymes are described in the scientific literature, for example, see Wood, R. D., et al. Mutat. Res. 577(1-2):275-83 (2005) and Eisen, J. A. and Hanawait, P. C. Mutat. Res. 435(3):171-213 (1999). A list of human repair enzymes is provided in Table 1 below. Although not described in Table 1, the homologs of the listed enzymes and other functionally related enzymes are included in the description of repair enzymes. Any of the above enzymes may be naturally occurring, recombinant or synthetic. Any of the enzymes may be a native or an in vitro-created chimeric protein with several activities. The methods of searching the databases to identify related enzymes that share conserved sequence motifs and have similar enzyme activity are known to a person of ordinary skill in the art. For example, the NCBI web site (www.ncbi.com) provides a conserved domain database. If, for example, the database is searched for homologs of endonuclease IV, 74 sequence matches are recovered. (Also see FIGS. 6A-1-6A-9 and 6B-1-6B-2 for DNA ligases).

A “polynucleotide cleavage enzyme” used in enzyme mixtures for repairing damaged DNA refers in particular to a class of repair enzymes and includes AP endonucleases, glycosylases and lyases responsible for base excision repair.

The AP endonuclease is characterized by an effective amount that contributes to repair without degrading the polynucleotide. AP endonucleases may have exonucleases associated with them. For example, exonuclease III was found to have significant degradative activity on DNA (see Fromenty et al. Nucl. Acids Res. 28(11):e50 (2000) and U.S. published application 2005-0026147. The effective amount is here defined as the amount of enzyme that cleaves specifically at AP sites on for example an oligonucleotide but does not show detectable amounts of non-specific degradation of the oligonucleotide as determined by standard gel electrophoresis. The effective amount of an AP endonuclease identified herein is in the range of 0.0001-100 units/μl. Beyond the upper limit of this range, non-specific degradation becomes a problem as determined for endonuclease VI. In the past, the activity of endonuclease VI was measured in units of exonuclease activity. The amount of exonuclease activity exceeded the upper limit of the endonuclease concentration provided herein.

A damaged base can be removed by a DNA glycosylase enzyme, which hydrolyses an N-glycosylic bond between the deoxyribose sugar moiety and the base. For example, an E. coli glycosylase and an UDG endonuclease act upon deaminated cytosine while two 3-mAde glycosylases from E. coli (TagI and TagII) act upon damage from alkylating agents.

The product of removal of a damaged base by a glycosylase is an AP site that must be correctly replaced. This can be achieved by an endonuclease, which nicks the sugar phosphate backbone adjacent to the AP site. The abasic sugar is removed and a new nucleotide is inserted by DNA polymerase/DNA ligase activity. These repair enzymes are found in prokaryotic and eukaryotic cells. In an embodiment, an AP endonuclease for use in the present universal mix should be used in the activity range specified and within this activity range, no inactivation step prior to amplification should be required. An AP endonuclease can be tested for its use in the present methods and compositions using the assay described in Example 20.

Some enzymes having applicability herein have glycosylase and AP endonuclease activity in one molecule. Abasic sites can be recognized and cleaved by AP endonucleases and/or AP lyases. Class II AP endonucleases cleave at AP sites to leave a 3′ OH that can be used in polynucleotide polymerization. Furthermore, AP endonucleases can remove moieties attached to the 3′ OH that inhibit polynucleotide polymerization. For example a 3′ phosphate can be converted to a 3′ OH by E. coli endonuclease IV. AP endonucleases can work in conjunction with glycosylases.

Examples of glycosylase substrates include Uracil, Hypoxanthine, 3-methyladenine (3-mAde), Formamidopyrimidine (FAPY), 7,8 dihydro-8-oxyguanine and Hydroxymethyluracil. The presence of uracil in DNA may occur due to mis-incorporation or deamination of cytosine by bisulfate, nitrous acids, or spontaneous deamination. Hypoxanthine generally occurs due to deamination of adenine by nitrous acids or spontaneous deamination. In this context, 3-mAde is a product of alkylating agents. FAPY (7-mGua) is a common product of methylating agents of DNA. 7,8-dihydro-8 oxoguanine is a mutagenic oxidation product of guanine. Gamma radiation produces 4,6-diamino-5-FAPY. Hydroxymethyuracil is created by ionizing radiation or oxidative damage to thymidine.

These different types of damage may be repaired using glycosylases of the sort described above and in Table 1.

Another type of repair enzyme is a lyase. This enzyme can break the phosphodiester bond in a polynucleotide.

Several enzymes have been isolated that appear to have AP endonuclease or lyase and glycosylase activities that are coordinated either in a concerted manner or sequentially.

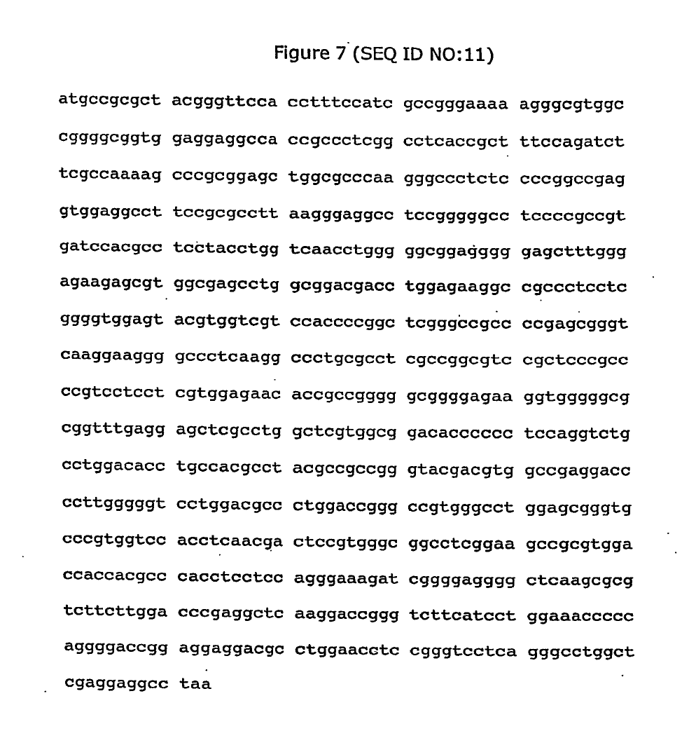

Examples of polynucleotide cleavage enzymes now found to be suitable for use in enhancing at least one of yield or fidelity in a copying or amplification reaction include the following types of enzymes derived from but not limited to any particular organism or virus: 1) AP endonucleases, such as E. coli endonuclease IV, Tth endonuclease IV (FIG. 7), and human AP endonuclease; 2) glycosylases, such as UDG, E. coli 3-methyladenine DNA glycoylase (AlkA) and human Aag; 3) glycosylase/lyases, such as E. coli endonuclease III, E. coli endonuclease VIII, E. coli Fpg, human OGG1, and T4 PDG; and 4) lyases.

Present embodiments of the method do not require inactivation of repair enzymes after repair and prior to amplification because endonuclease VI type degradation described in the prior art is avoided by using this enzyme at a lower concentration than previously described, ie in the range.

A “DNA polymerase” for present purposes refers to an enzyme that has DNA polymerase activity even though it may have other activities. A single DNA polymerase or a plurality of DNA polymerases may be used throughout the repair and copying reactions. The same DNA polymerase or set of DNA polymerases may be used at different stages of the present methods or the DNA polymerases may be varied or additional polymerase added after repair for subsequent manipulations. Polymerases include hyperthermophilic enzymes such as Vent® polymerase and Taq DNA polymerase, thermophilic enzymes such as Bst DNA polymerase and mesophilic polymerases. Polymerases from any of these three groups of enzymes may be used herein. Preferably gap filling polymerases or nick-translating polymerases in these groups are used in the present embodiments. An effective amount of DNA polymerase can be readily ascertained by titrating the DNA polymerase with a fixed concentration of DNA ligase and AP endonuclease using a know DNA such as described in the Examples.

Examples of polymerases include thermostable bacterial polymerases such as Taq DNA and Tth polymerases and archeal polymerases such as Vent®, Deep Vent™ and Pfu; less thermostable enzymes such as Bst polymerase, thermomicrobium roseum DNA polymerases and mesophilic DNA polymerases such as some phage DNA polymerases (such as phi29 DNA polymerase, T7 DNA polymerase and T4 DNA polymerase), E. coli pol I and E. coli pol II Y family DNA polymerases such as E. coli pol IV, E. coli pol V, human pol kappa, human pol eta, Sso Dpo 4, Sac Dbh, Sce pol zeta, human pol iota (MacDonald et al. Nucleic Acids Res. 34:1102-1111 (2006); Vaisman et al. DNA Repair 5:210 (2006); Ohmori et al. Mol. Cell. 8:7-8 (2001); Goodman Ann. Rev. Biochem. 71:17-50 (2002)) or mutants, derivatives or modifications therefrom. Examples of derivatives include Phusion™ enzyme (Finnzymes, Espoo, Finland) and other DNA polymerases that combine a double strand binding protein with polymerase sequences from one or several sources.

A “DNA ligase” as used in the enzyme mixtures described here refers to an enzyme that joins a 5′ end of a single strand of a polynucleotide to a 3′ end of another single strand of a polynucleotide. An effective amount of ligase is an amount generally used in biochemical applications. There are limited or no adverse consequences of using an excess of DNA ligase in a repair reaction. Such DNA ligases are found in substantially all eukaryotic, prokaryotic, and archaeal cells, and can also be found in some bacteriophages and viruses. Examples of suitable DNA ligases include 9° N DNA ligase (PCT/US06/35919), E. coli DNA ligase, and Taq DNA ligase. T4 DNA ligase may also be used under limited circumstances. This DNA ligase efficiently blunt ends DNA giving rise to undesirable chimeras during subsequent amplification or copying steps.

Other DNA ligases or DNA ligase-like proteins that may have utility herein are revealed by a Blast search using, for example, E. coli DNA ligase to search the database (see FIGS. 6A-1-6A-9 and FIGS. 6B-1 and 6B-2) in which any enzyme sharing at least 6 contiguous amino acids with these known DNA ligases may be included in a repair mixture according to embodiments of the invention.

Contrary to a published use of DNA ligase in combination with exonuclease III in the absence of any cofactors (U.S. Publication No. 2005-0026147), it has been found here that NAD⁺ or ATP is required in enzyme mixtures that include DNA ligase. More specifically, Taq DNA ligase and E. coli DNA ligase require NAD⁺ while 9° N DNA ligase and T4 DNA ligase require ATP. FIGS. 20A and 20B show how ATP at concentrations of greater than 500 μM interferes with amplification.

Certain DNA ligases, DNA polymerases and endonucleases are available from NEB, Ipswich, Mass. where pages 107-117 of the 2005-2006 catalog are incorporated by reference (pp. 102-108 for DNA ligases) and described in International Application No. PCT/US06/35919 and International Publication No. WO 2005/052124. In addition, thermostable repair enzymes can be used interchangeably with thermolabile repair enzymes in a pre-amplification mixture. Thermostable enzymes retain activity at above 40° C. or more particularly 65° C. or above.

Unit definitions of enzymes exemplified in the universal mix are as follows:

(a) Thermophilic UDG

One unit is defined as the amount of enzyme that catalyzes the release of 60 pmol of uracil per minute from double-stranded, uracil-containing DNA. Activity is measured by release of [3H]-uracil in a 50 μl reaction containing 0.2 μg DNA (104-105 cpm/μg) in 30 minutes at 65° C. (Reaction buffer: 10 mM KCl, 10 mM (NH₄)₂SO₄, 20 mM Tris-HCl, 0.1% Triton X-100, pH 8.8 at 25° C.)

(b) Mesophilic UDG

One unit is defined as the amount of enzyme that catalyzes the release of 60 pmol of uracil per minute from double-stranded, uracil-containing DNA. Activity is measured by release of [3H]-uracil in a 50 μl reaction containing 0.2 μg DNA (104-105 cpm/μg) in 30 minutes at 37° C. (Reaction buffer: 20 mM KCl, 1 mM EDTA, 1 mM DTT, pH 8.0 at 25° C.)

(c) Mesophilic or Thermophilic Endonuclease VIII

One unit is defined as the amount of enzyme required to cleave 1 pmol of a 34 mer oligonucleotide duplex containing a single AP site in a total reaction volume of 10 μl in 1 hour at 37° C. in 1×Endonuclease VIII Reaction Buffer containing 10 pmol of fluorescently labeled oligonucleotide duplex. (An AP site is created by treating 10 pmol of a 34 mer oligonucleotide duplex containing a single uracil residue with 1 unit of UDG for 2 minutes at 37° C.; reaction buffer: 10 mM Tris-HCl, 75 mM NaCl, 1 mM EDTA, pH 8.0 at 25° C.)

(d) Mesophilic or Thermophilic Endonuclease III

One unit is defined as the amount of enzyme required to cleave 1 pmol of a 34 mer oligonucleotide duplex containing a single AP site in a total reaction volume of 10 μl in 1 hour at 37° C. in 1×endonuclease III Reaction Buffer containing 10 pmol of fluorescently labeled oligonucleotide duplex. (Reaction buffer: 20 mM Tris-HCl, 1 mM EDTA, 1 mM DTT, pH 8.0 at 25° C.)

(e) Mesophilic or Thermophilic Fpg

One unit is defined as the amount of enzyme required to cleave 1 pmol of a 34 mer oligonucleotide duplex containing a single 8-oxoguanine base paired with a cytosine in a total reaction volume of 10 μl in 1 hour at 37° C. in 1× NEBuffer 1 (NEB, Ipswich, Mass.) with 10 pmol of fluorescently labeled oligonucleotide duplex. (Reaction buffer: 10 mM Bis-Tris-Propane-HCl, 10 mM MgCl2, 1 mM Dithiothreitol, pH 7.0 at 25° C., and 0.1 mg/mL BSA.)

(f) Mesophilic or Thermophilic 8-oxoguanine DNA glycosylase

One unit is defined as the amount of enzyme required to cleave 1 pmol of a 34 mer oligonucleotide duplex containing a single 8-oxoguanine base paired with a cytosine in a total reaction volume of 10 μl in 1 hour at 37° C. in 1× NEBuffer 2 (NEB, Ipswich, Mass.) containing 10 pmol of fluorescently labeled oligonucleotide duplex. (Reaction buffer: 10 mM Tris-HCl, 50 mM NaCl, 10 mM MgCl₂, 1 mM Dithiothreitol, pH 7.9 at 25° C., and 0.1 mg/mL BSA.)

(g) Mesophilic or Thermophilic PDG

One unit is defined as the amount of enzyme that catalyzes the conversion of 0.5 μg of UV-irradiated, supercoiled pUC19 DNA to greater than 95% nicked plasmid in a total reaction volume of 20 μl in 30 minutes at 37° C. Nicking is assessed by agarose gel electrophoresis. Irradiated plasmid contains an average of 3-5 pyrimidine dimers. (Reaction buffer: 100 mM NaCl, 1 mM DTT, 1 mM EDTA, 25 mM Na₂HPO₄, pH 7.2 at 25° C., and 0.1 mg/mL BSA.)

(h) E. coli Endonuclease V

One unit is defined as the amount of enzyme required to cleave 1 pmol of a 34 mer oligonucleotide duplex containing a single deoxyinosine site in a total reaction volume of 10 μl in 1 hour at 37° C. (A deoxyinosine site is synthetically prepared with a deoxyinosine in the middle of one strand of a 34 mer oligonucleotide duplex; reaction buffer: 20 mM Tris-acetate, 50 mM potassium acetate, 10 mM Magnesium Acetate, 1 mM Dithiothreitol, pH 7.9 at 25° C.)

(i) Thermophilic DNA Ligase

One unit is defined as the amount of enzyme required to give 50% ligation of 1 μg of BstE II-digested lambda DNA in a total reaction volume of 50 μl in 15 minutes at 45° C. Taq DNA ligase is available from NEB, Ipswich, Mass. (Reaction buffer: 20 mM Tris-HCl, 25 mM potassium acetate, 10 mM Magnesium Acetate, 10 mM Dithiothreitol, 0.1% Triton X-100, pH 7.6 at 25° C. Either 1 mM ATP or 0.5 mM NAD⁺ is included in the reaction depending on the co-factor requirement of the ligase.)

(j) Mesophilic DNA Ligase

One unit is defined as the amount of enzyme required to give 50% ligation of Hind III digested lambda DNA (5′ DNA termini concentration of 0.12 μM, 300 μg/ml) in a total reaction volume of 20 μl in 30 minutes at 16° C. E. coli DNA ligase is available from NEB, Ipswich, Mass. (Reaction buffer: 30 mM Tris-HCl, 4 mM MgCl2, 1 mM Dithiothreitol, 50 μg/ml BSA, pH 8.0 at 25° C. Either 1 mM ATP or 0.5 mM NAD⁺ is included in the reaction depending on the co-factor requirement of the ligase.)

(k) AP Endonuclease

One unit is defined as the amount of enzyme required to cleave 1 pmol of a 34-mer oligonucleotide duplex containing a single AP site in a total reaction volume of 10 μl in 1 hour at 37° C. (Reaction buffer: 50 mM Tris-HCl, 100 mM NaCl, 10 mM MgCl₂, 1 mM Dithiothreitol, pH 7.9 at 25° C.)

(i) Mesophilic DNA Polymerase

One unit is defined as the amount of enzyme that will incorporate 10 nmol of dNTP into acid-insoluble material in a total reaction volume of 50 μl in 30 minutes at 37° C. with 33 μM dNTPs including [³H]-dTTP and 70 μg/ml denatured herring sperm DNA. (Reaction buffer: 10 mM Tris-HCl, 50 mM NaCl, 10 mM MgCl₂, 1 mM Dithiothreitol, pH 7.9 at 25° C.)

(j) Thermophilic DNA Polymerase

One unit is defined as the amount of enzyme that will incorporate 10 nmol of dNTP into acid-insoluble material in a total reaction volume of 50 μl in 30 minutes at 75° C. with 200 μM dNTPs including [3H]-dTTP and 200 μg/ml activated Calf Thymus DNA. Thermophilic DNA polymerases-Taq polymerase and archaeal DNA polymerases are available from NEB, Ipswich, Mass.

The unit definitions for thermophilic UDG, Fpg, endonuclease III and endonuclease VIII are the same as those for the mesophilic equivalents listed (NEB catalog, NEB, Ipswich, Mass.). (Reaction buffer: 20 mM Tris-HCl, 10 mM (NH₄)₂SO₄, 10 mM KCl, 2 mM MgSO₄, 0.1% Triton X-100, pH 8.8 at 25° C.

| Gene Name | Activity | Accession Number |
|---|---|---|
| UNG | Uracil-DNA glycosylase | NM_080911 |
| SMUG1 | Uracil-DNA glycosylase | NM_014311 |
| MBD4 | Removes U or T opposite G | NM_003925 |
| | at CpG sequences | |
| TDG | Removes U, T or ethenoC | NM_003211 |
| | opposite G | |
| OGG1 | Removes 8-oxoG opposite C | NM_016821 |
| MUTYH | Removes A opposite 8-oxoG | NM_012222 |
| (MYH) | | |
| NTHL1 | Removes Ring-saturated or | NM_002528 |
| (NTH1) | fragmented pyrimidines | |
| MPG | Removes 3-meA, ethenoA, | NM_002434 |
| | hypoxanthine | |
| NEIL1 | Removes thymine glycol | NM_024608 |
| NEIL2 | Removes oxidative products | NM_145043 |
| | of pyrimidines | |
| XPC | Binds damaged DNA as complex | NM_004628 |
| RAD23B | XPC, RAD23B, CETN2 | NM_002874 |
| (HR23B) | | |
| CETN2 | | NM_004344 |
| RAD23A | Substitutes for HR23B | NM_005053 |
| (HR23A) | | |
| XPA | Binds damaged DNA in | NM_000380 |
| | preincision complex | |
| RPA1 | Binds DNA in preincision | NM_002945 |
| RPA2 | complex | NM_002946 |
| RPA3 | RPA1, RPA2, RPA3 | NM_002947 |
| ERCC5 | 3′ incision | NM_000123 |
| (XPG) | | |
| ERCC1 | 5′ incision subunit | NM_001983 |
| ERCC4 | 5′ incision subunit | NM_005236 |
| (XPF) | | |
| LIG1 | DNA joining | NM_000234 |
| CKN1 | Cockayne syndrome; Needed | NM_000082 |
| (CSA) | for | |
| ERCC6 | transcription-coupled NER | NM_000124 |
| (CSB) | CKN1, ERCC6, XAB2 | |
| XAB2 | | NM_020196 |
| (HCNP) | | |
| DDB1 | Complex defective in XP | NM_001923 |
| | group E | |
| DDB2 | DDB1, DDB2 | NM_000107 |
| MMS19L | Transcription and NER | NM_022362 |
| (MMS19) | | |
| FEN1 | Flap endonuclease | NM_004111 |
| (DNase IV) | | |
| SPO11 | endonuclease | NM_012444 |
| FLJ35220 | incision 3′ of hypoxanthine | NM_173627 |
| (ENDOV) | and uracil | |
| FANCA | Involved in tolerance or | NM_000135 |
| FANCB | repair of DNA crosslinks | NM_152633 |
| FANCC | FANCA, FANCB, FANCC, | NM_000136 |
| FANCD2 | FANCD2, FANCE, | NM_033084 |
| FANCE | FANCF, FANCG, FANCL | NM_021922 |
| FANCF | | NM_022725 |
| FANCG | | NM_004629 |
| (XRCC9) | | |
| FANCL | | NM_018062 |
| DCLRE1A | DNA crosslink repair | NM_014881 |
| (SNM1) | | |
| DCLRE1B | Related to SNM1 | NM_022836 |
| (SNM1B) | | |
| NEIL3 | Resembles NEIL1 and NEIL2 | NM_018248 |
| ATRIP | ATR-interacting protein | NM_130384 |
| (TREX1) | 5′ alternative ORF of the | |
| | TREX1/ATRIP gene | |
| NTH | Removes damaged pyrimidines | NP_416150.1 |
| NEI | Removes damaged pyrimidines | NP_415242.1 |
| NFI | Deoxyinosine 3′ endonuclease | NP_418426.1 |
| MUTM | Formamidopyrimidine DNA | NP_418092.1 |
| | glycosylase | |
| UNG | Uracil-DNA glycosylase | NP_417075.1 |
| UVRA | DNA excision repair enzyme | NP_418482.1 |
| UVRB | complex | NP_415300.1 |
| UVRC | UVRA, UVRB, UVRC | NP_416423.3 |
| DENV | Pyrimidine dimer glycosylase | NP_049733.1 |

Examples of concentrations of enzymes in a universal mixture of enzymes are: T4 PDG in a concentration range of 0.0001 units/μl to 4 units/μl, Taq DNA ligase in a concentration range of 0.00001 units/μl to 100 units/μl, Bst DNA polymerase in a concentration range of 0.00001 units/μl to 2 units/μl, E. coli endonuclease IV in the range of 0.0001 units/μl to 100 units/μl, endonuclease VIII in the range of 0.00001 units/μl to 20 units/μl, UDG in the range of 0.00001 units/μl to 20 units/μl, and Fpg in the range of 0.000001 units/μl to 0.1 units/μl.

The concentration range for endonucleases and DNA polymerases other than those specified in the universal mixture above may vary with the enzyme used and the temperature of the reaction. However, the concentration range can be readily ascertained using the assays described in the Examples. For example, a standard preparation of lambda DNA can be heat-treated according to Example 1. The DNA can then be subjected to a series of enzyme mixtures containing DNA ligase and cofactors. An additional enzyme is titrated to determine a preferred concentration for that enzyme in the mixture. In this way, DNA repair can be optimized. After amplification of each sample, the amount of the amplified DNA can be determined by gel electrophoresis revealing the preferred concentration range for the test enzyme.

As illustrated in the Examples, depending on the type of damage, it may be desirable to supplement the universal enzyme mixture with additional repair enzymes depending on the nature of the DNA damage. The utility of individual repair enzymes or mixtures of repair enzymes can be determined using the assays described in the Examples and in the Figures to determine their suitability for repairing a particular polynucleotide.

### Repair of General or Specific Damage to Polynucleotides

(a) General damage

Determining the nature of damage in a polynucleotide is time-consuming. If some form of damage to a polynucleotide is suspected, for example, the polynucleotide is poorly amplified, it is preferable not to have to identify the lesion or lesions. In these circumstances, a universal mix of enzymes such as described above may be utilized to determine whether improved amplification is obtained. If the improvement is sufficient using the universal mixture then no further action is required. If the improvement is not sufficient, additional enzymes can be added to the mixture as described herein until the preferred result is obtained. The entire assay may be achieved in a single reaction vessel such as a 96 well dish. Each micro-well in the dish is available for a different enzyme mixture including the universal mixture plus enzymes selected to address each class of damage outlined below.

The protocol for selecting enzymes for repair of general damage or unknown damage of DNA is provided in FIG. 12 (flow chart) and in the assays described in the Examples.

### (b) Specific Damage

(i) AP Sites

The loss of a base is the most common form of spontaneous DNA damage under physiological conditions. DNA polymerases and DNA polymerase-based techniques are adversely affected by the presence of these abasic sites. The effectiveness of primer extension reactions is enhanced by repairing any abasic sites found in a polynucleotide. This is achieved in one embodiment by endonuclease IV activity that cleaves the phosphate backbone at the abasic site. This leaves an extendable 3′ OH on the DNA fragment 5′ to the cleaved abasic site. It also leaves a deoxyribose-5′-phosphate (dR5P) on the DNA fragment 3′ to the cleaved abasic site. A DNA polymerase can extend from the free 3′ OH replacing the cleaved abasic site with a correct nucleotide. The dR5P may be removed by an enzyme that specifically targets dR5Ps such as mammalian pol beta or the 8 Kd N-terminal portion of mammalian pol beta (Deterding J Biol Chem 275:10463-71 (2000)), by a flap endonuclease activity present in certain DNA polymerases such as E. coli DNA polymerase I or by a separate flap endonuclease such as FENI. The removal of dR5P can also occur by cleavage downstream of this group by the flap endonuclease activity. After removal of the dR5P and the generation of a 5′ phosphate adjacent to the 3′ OH, a DNA ligase can seal this nick finishing the repair (see Examples 1-3).

(ii) Modified Nucleotides

(a) Thymidine Dimers

Light can damage DNA by inducing the formation of pyrimidine dimers. Pyrimidine dimers block the DNA extension reaction catalyzed by DNA polymerases such as Taq DNA polymerase and hence inhibit DNA amplification (Wellinger, et al. Nucleic Acids Res. 24(8):1578-79 (1996)). Consequently, it is desirable to repair pyrimidine dimers prior to or during amplification. This can be achieved by adding a pyrimidine dimer glycosylase/lyase (Vande Berg, et al. J. Biol. Chem. 273(32):20276-20284 (1998)) to the universal enzyme mixture. The DNA backbone is cleaved 5′ to the pyrimidine dimer and leaves a 3′ hydroxyl moiety that is extendable by a DNA polymerase. In certain embodiments, extension at the 3′ hydroxyl and subsequent formation and then cleavage of the lesion-containing flap generated during DNA extension results in a nick that is sealed by an enzyme capable of sealing the nick. Cleavage of the flap can be achieved by the extending DNA polymerase, for example, E. coli DNA polymerase I or by the action of a flap endonuclease ((Xu, Y., et al. J. Biol. Chem. 275(27):20949-20955 (2000), Liu, Y., et al., Annu. Rev. Biochem. 73:589-615 (2004)).

(b) Oxidative Damage, Alkylation and Deamination

Inaccuracies can be introduced into the products of DNA amplification reactions because of undesired nucleotide incorporation opposite a damaged base (Gilbert, et al. Am. J. Hum. Gen. 72:48-61 (2003); Hofreiter et al. Nucl. Acids Res. 29:4793-9 (2001)). These inaccuracies can be discovered after amplifying, cloning and sequencing the same sample many times. Inaccuracies due to base damage can also be identified by comparing sequence data before and after sample treatment with an enzyme such as UDG, which removes one of the common types of mutagenic DNA lesions (Hofreiter, et al. Nucl. Acids Res 29:4793-9 (2001)). However, treatment with UDG creates an abasic site within the DNA that inhibits DNA amplification by primer extension. This may cause DNA samples to be refractory to amplification after UDG treatment. This AP site can then be repaired by a reaction mixture containing a DNA ligase and preferably also an AP endonuclease and a DNA polymerase. Removal of a uracil enables a DNA polymerase in an amplification reaction that would normally be stopped at this site to continue amplifying the DNA. For example, Vent® DNA polymerase activity is inhibited on DNA templates containing uracil. The ability to remove the uracil permits the DNA polymerase to have enhanced effectiveness.

In contrast, it is here shown that including UDG with an enzyme mixture that includes a DNA ligase and a DNA polymerase can be successfully used to enhance the yield and fidelity of the product of polynucleotide copying or amplification. Examples 11-13 provide descriptions of various beneficial enzyme mixes that include UDG.

Modified nucleotides that are the product of oxidative damage can also be removed from the polynucleotide by Fpg or hOGG to leave a blocked polynucleotide where the blocked polynucleotide is repairable by an AP endonuclease such as endonuclease IV.

The effectiveness of enzyme pretreatment to repair oxidative damage to a polynucleotide prior to copying or amplification is illustrated in Example 9 in which improved fidelity of the copied polynucleotide product is demonstrated using an enzyme mixture containing a DNA ligase, a DNA polymerase, endonuclease IV and Fpg.

Other modified nucleotides such as alkylated bases or deaminated bases where cytosine is converted to uracil, guanine to xanthine or adenine to hypoxanthine give rise to miscoding.

Removal of these modified nucleotides is desirable. These modified bases can be removed by UDG as discussed above or by AlkA or Aag as described in Example 10.

(iii) Cross-Links

Additional nucleotide excision repair (NER) proteins (Minko et al. Biochemistry 44:3000-3009 (2005); Costa et al. Biochimie 85(11):1083-1099 (2003); Sancar Ann. Rev. Biochem 65:43-81 (1996)) can be used to repair damage resulting from formaldehyde and bulky adducts as well as damage that results in chemically-modified bases that form DNA-protein cross-links. At least one of E. coli UvrA, UvrB, mutant UvrB, UvrC, UvrD or Cho (Moolenar et al. Proc. Natl Acad. Sci. USA 99:1467-72 (2002)) can be used to make incisions at the 5′ end and optionally the 3′ end around a damaged site. Details about the properties and purification protocols of these enzymes can be obtained from Zou, Y., et al. Biochemistry 43:4196-4205 (2004). The repair process can be completed by means of a DNA polymerase, a DNA ligase and optionally a flap endonuclease.

The generation of a 3′ hydroxyl at a 5′ incision, site can be useful if the NER enzyme(s) cleave the DNA but leave a blocked 3′ end on the DNA that inhibits primer extension. An example would be if the NER enzyme(s) cleaved the DNA and left a 3′ phosphate. This would not be extendable by known DNA polymerases unless the 3′ phosphate was removed by, for example, E. coli endonuclease IV.

If the NER enzyme or enzymes cleaves 5′ and 3′ to the DNA lesion, then the damage is removed when the newly released oligonucleotide dissociates from the DNA. A DNA polymerase can simply fill in the excised region of DNA leaving a nick, which DNA ligase then seals to complete the repair. In certain cases, the DNA polymerase may fill in the DNA and then proceed to displace the remaining DNA strand. In these circumstances, an enzyme with flapase activity permits a nick to be formed that a DNA ligase can seal. In cases in which the NER enzyme or enzymes only cleaves 5′ to the damage, the DNA polymerase preferably displaces the original DNA strand until it is past the damage, at which point a flapase cleaves the DNA flap to create a ligatable nick. Preferably, the DNA polymerase and flapase activities work to eventually displace and remove the DNA lesion leaving a ligatable nick, thus repairing the DNA template. An example of the effectiveness of the above approach is provided in Example 7.

(iv) Nicks, Gaps and Mismatched Polynucleotides

Nicks and gaps in the DNA backbone can lead to truncated primer extension products and formation of chimera by undesirable hybridization of single-strand regions. Heteroduplex DNA can be a problem in multi-template PCR and in homogeneous template PCR (Lowell, J. L. & Klein, D. A. Biotechniques 28:676-681 (2000); Thompson, J. R., et al. Nucl. Acids Res. 30(9):2083-2088 (2002); Smith, J. & Modrich, P. Proc. Natl. Acad. Sci. USA 94:6847-6850 (1997)). For example, chimera can be formed at the mismatch sites. ATP-dependent ligases such as T4 DNA ligase efficiently blunt end DNA also making chimera formation more likely during amplification.

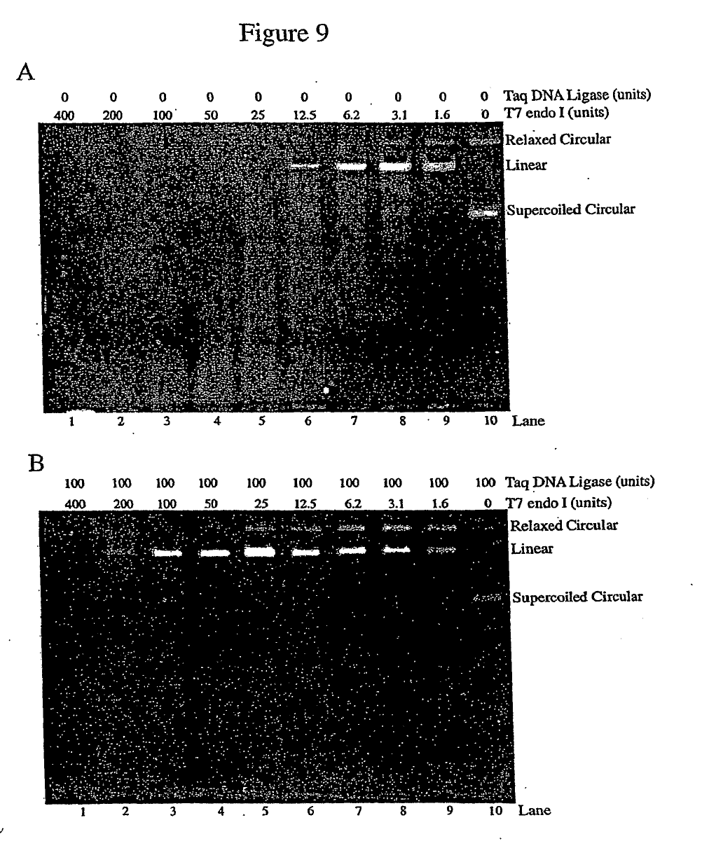

The combined effect of a DNA ligase and a DNA polymerase together optionally with an enzyme that recognizes and cleaves at heteroduplex sites (T7 endonuclease I and mutants thereof) contained within a universal enzyme mixture results in repairs of nicks, gaps, heteroduplexes and chimera in the DNA thus enhancing yield and fidelity of polynucleotide-copying and amplification reactions. Example 8 and FIG. 9 demonstrates the beneficial effects of using T7 endonuclease I and a DNA ligase as illustrative of the above. Addition of a DNA ligase to a reaction mixture containing T7 endonuclease I or mutant thereof permits the use of increased concentrations of the endonuclease without non-specific degradation of DNA. This approach does not require quantitation of DNA and avoids the extra steps after the PCR reaction required by Lowell, et al. Biotechniques 28:676-681 (2000); and Smith, et al. Proc. Natl. Acad. Sci. USA 94:6847-6850 (1997).

For some polynucleotides, the nature of the damage might be known. By way of illustration, a mixture of enzymes can be selected according to section (b) above for repairing the specific damage. Where the damage is unknown or the sources are mixed, the universal mix described herein including in the Examples may be employed.

### DNA Microarrays

DNA microarrays are a powerful methodology used to analyze DNA samples (Lipshutz et al. Curr Opinion in Structural Biology 4:376-380 (1994); Kozal, et al. Nat Med 2(7):753-9 (1996)). The amount and quality of information from microarray analysis of damaged DNA would benefit from first repairing the damaged DNA.

### Amplification

Where polynucleotide-copying leads to DNA polymerase-dependent amplification, short amplicons that are less than about 500 bases in length (as short as 100 base pairs) or long amplicons that are greater than 500 bases or as much as about 100 kb may be amplified (for PCR, RT-PCR and qPCR amplification). Other types of amplification can produce amplicons having a wide range of sizes. For example, polynucleotides having a size as small as 100 bases or as large as a whole genome (3 billion bases for humans) can be amplified. The limitation of size of amplicon is determined by the amplification protocol.

Pre-incubation of a sample polynucleotide using methods described herein improve the reproducibility and accuracy of the amplified product. Amplification protocols that benefit from the above described pre-incubation include PCR, Strand-Displacement Amplification (SDA) (U.S. Pat. Nos. 5,455,166 and 5,470,723); Helicase-Dependent Amplification (HDA) (U.S. Publication No. 2004-0058378-A1); Transcription-Mediated Amplification (TMA) (Guatelli et al., Proc. Natl. Acad. Sci. USA 87:1874-1878 (1990)); Rolling Circle Amplification (RCA) which generates multiple copies of a sequence for the use in in vitro DNA amplification adapted from in vivo rolling circle DNA replication (see, for example, Fire and Xu, Proc. Natl. Acad. Sci. USA 92:4641-4645 (1995); Lui, et al., J. Am. Chem. Soc. 118:1587-1594 (1996); Lizardi, et al., Nature Genetics 19:225-232 (1998)); and whole genome amplification methods. (Hawkins et al. Current Opinions in Biotechnology 13:65-67 (2002)). Other types of polynucleotide synthesis include primer extension reactions such as sequencing reactions.

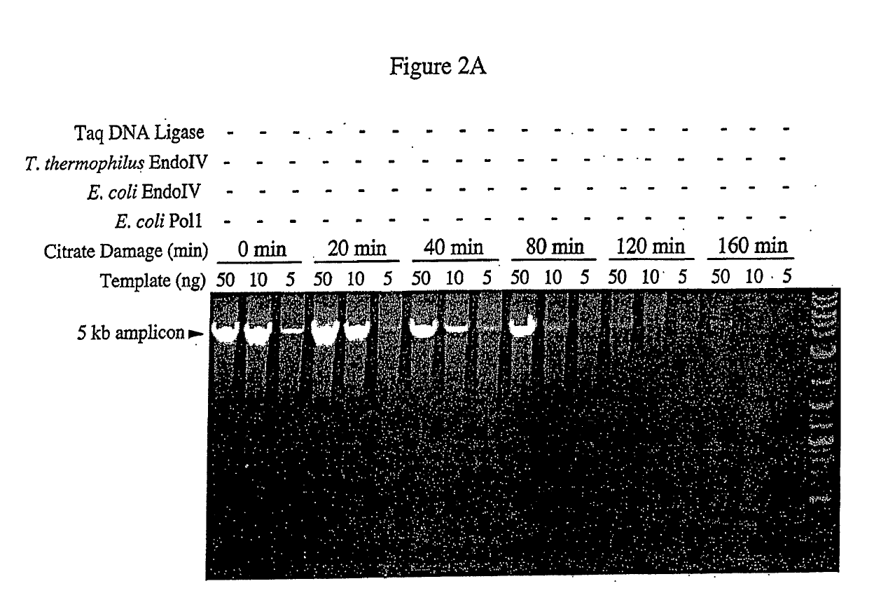

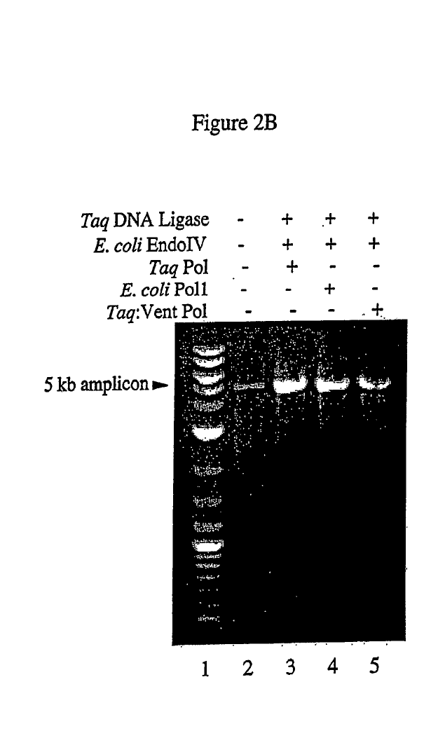

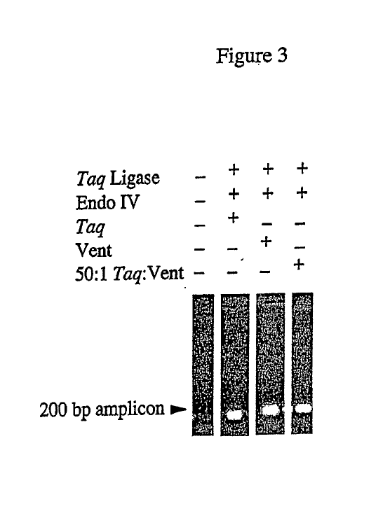

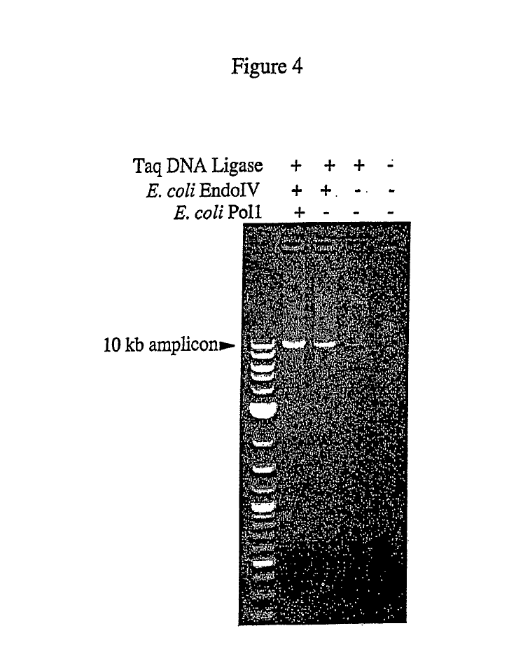

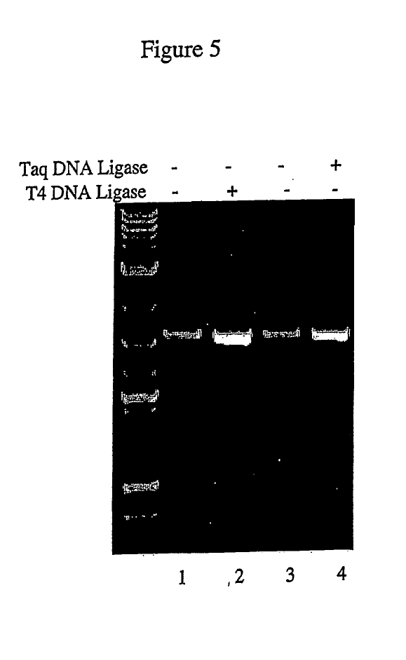

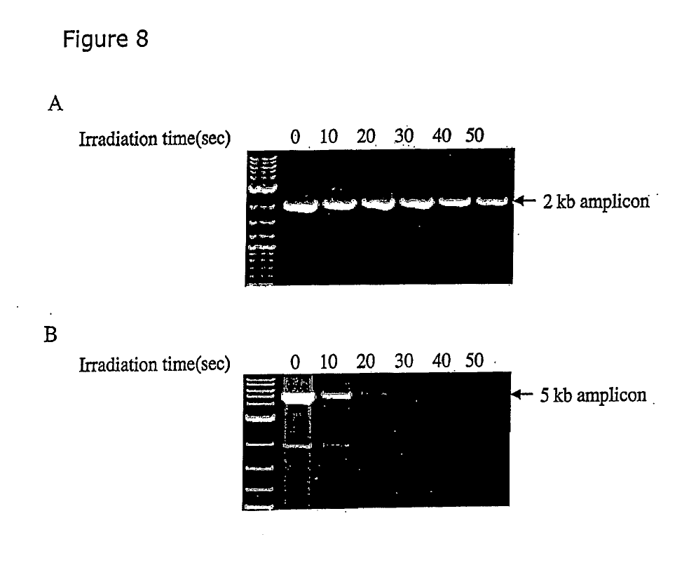

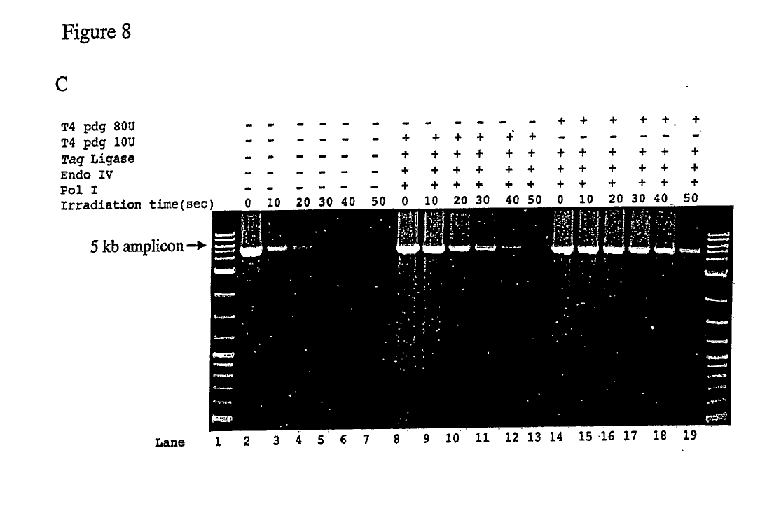

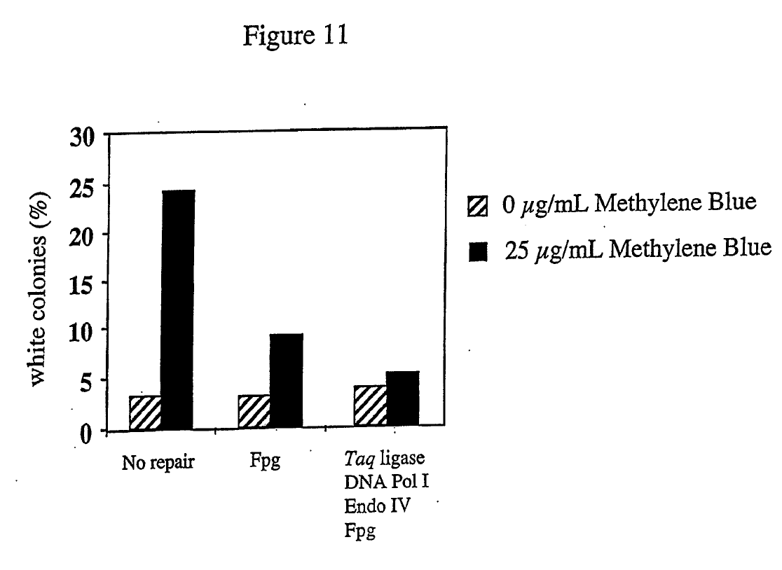

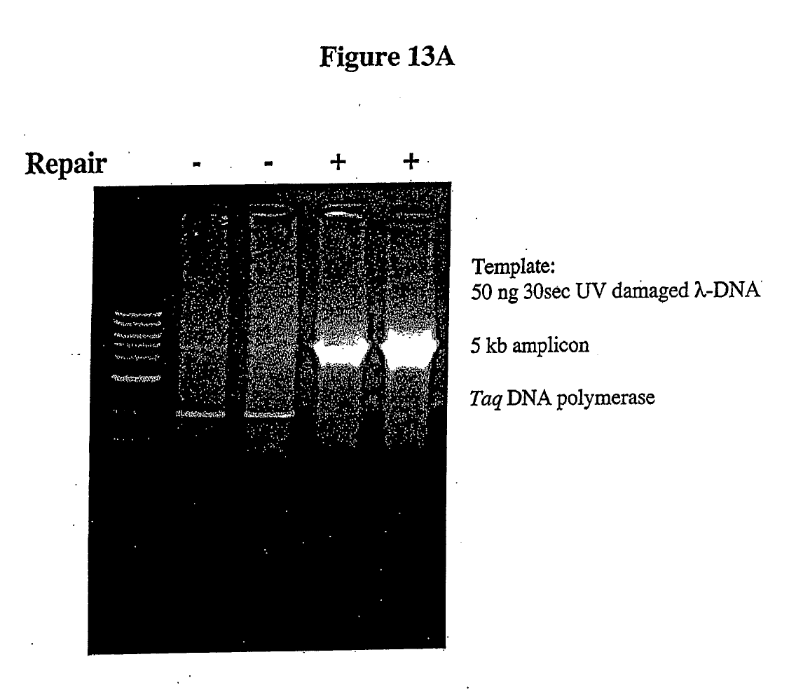

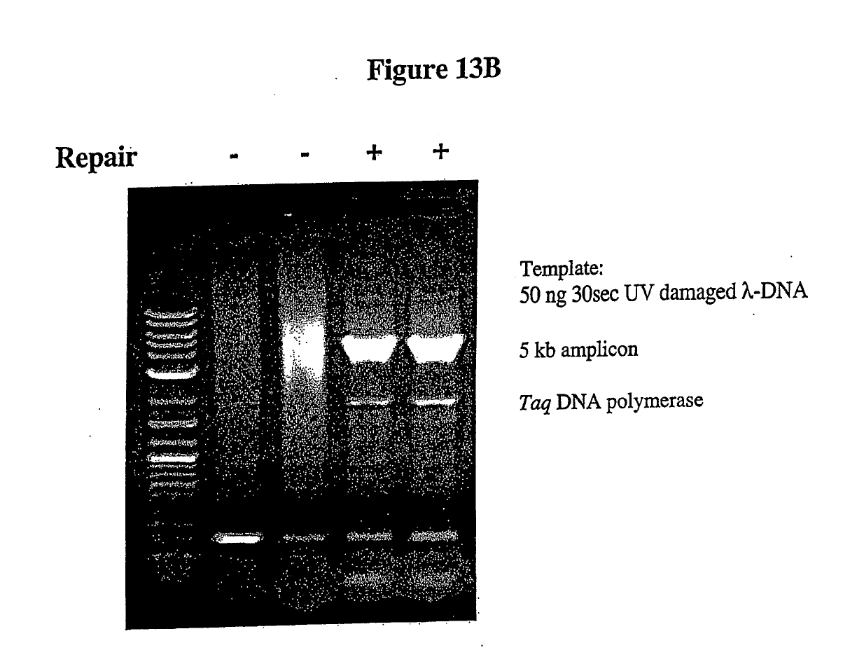

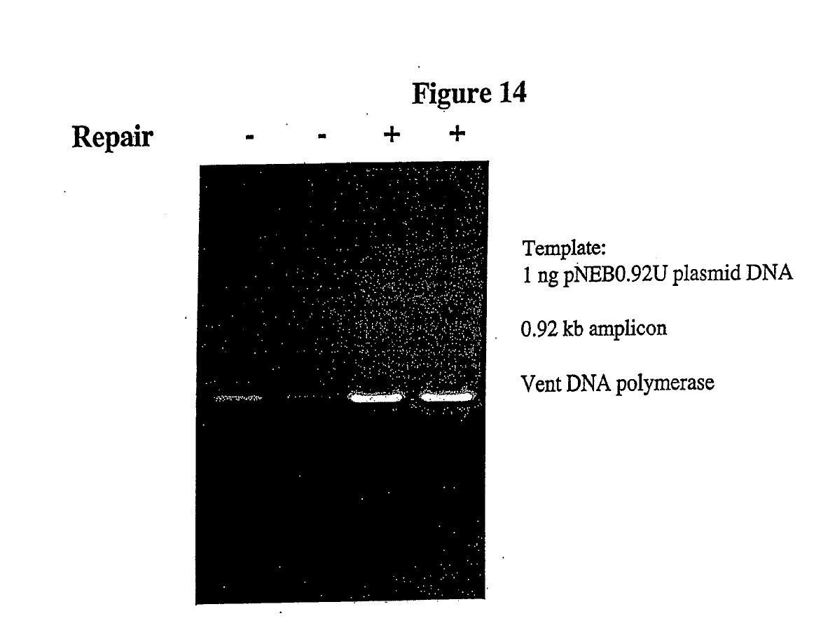

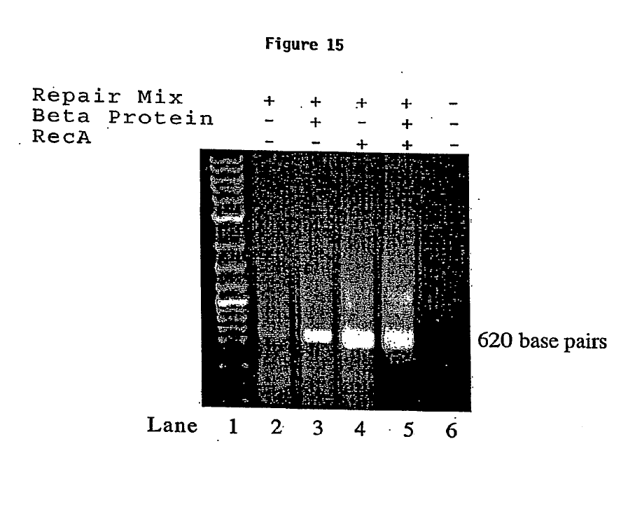

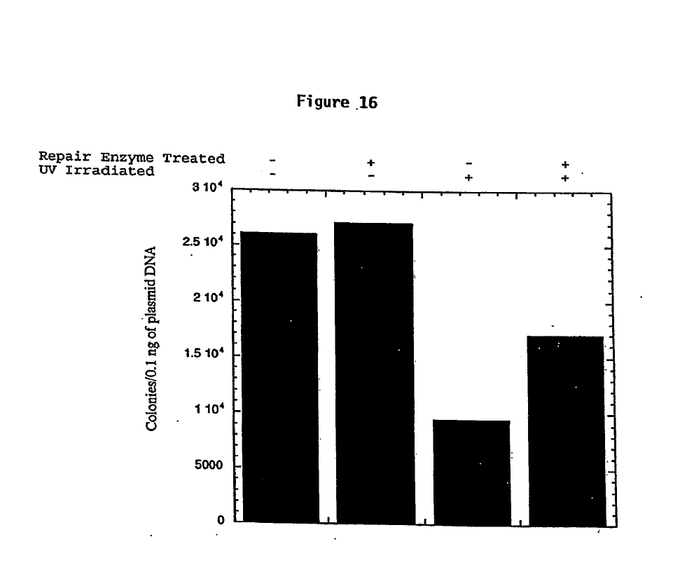

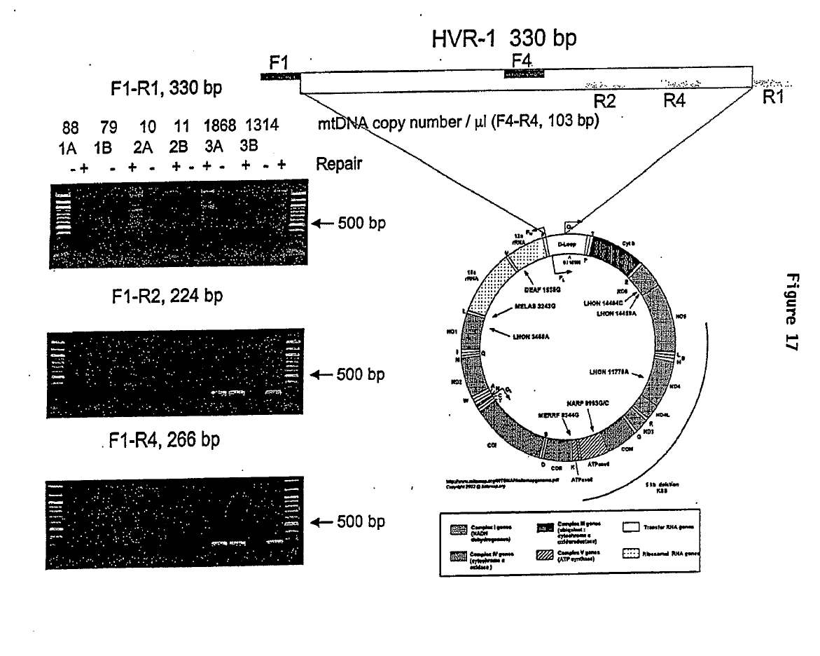

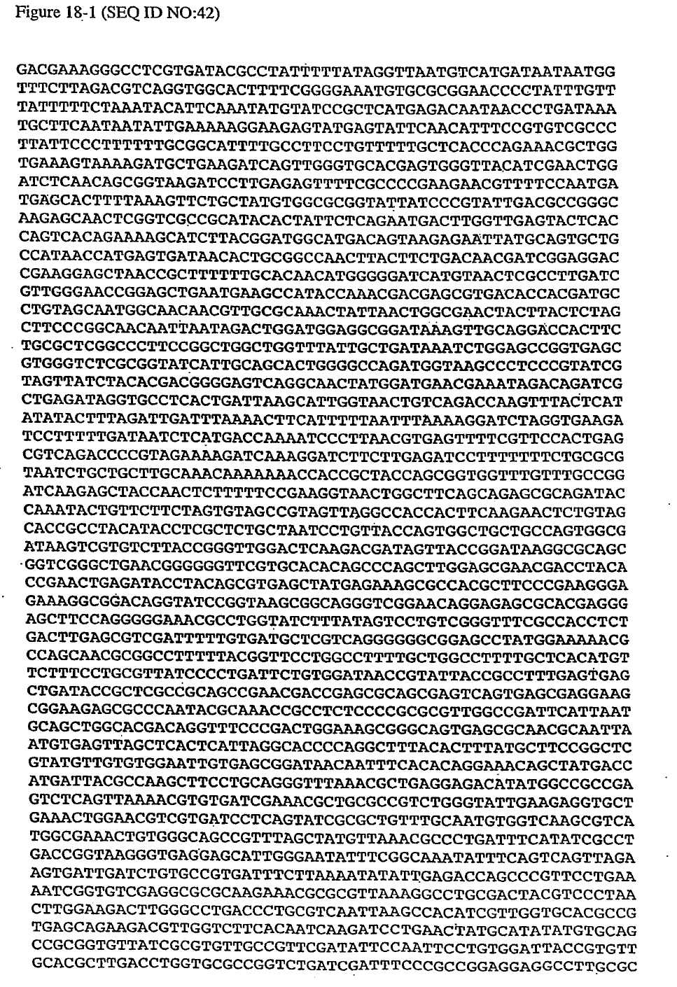

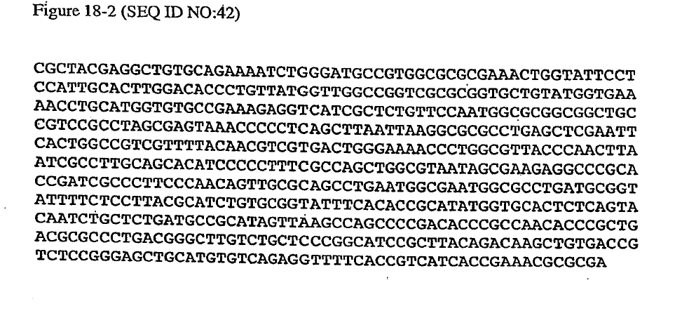

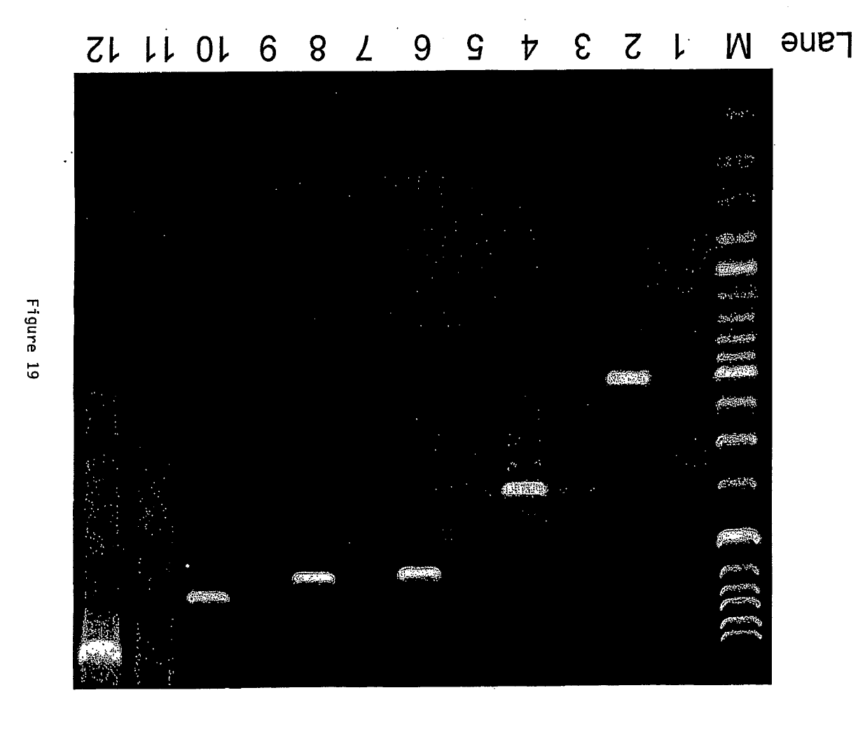

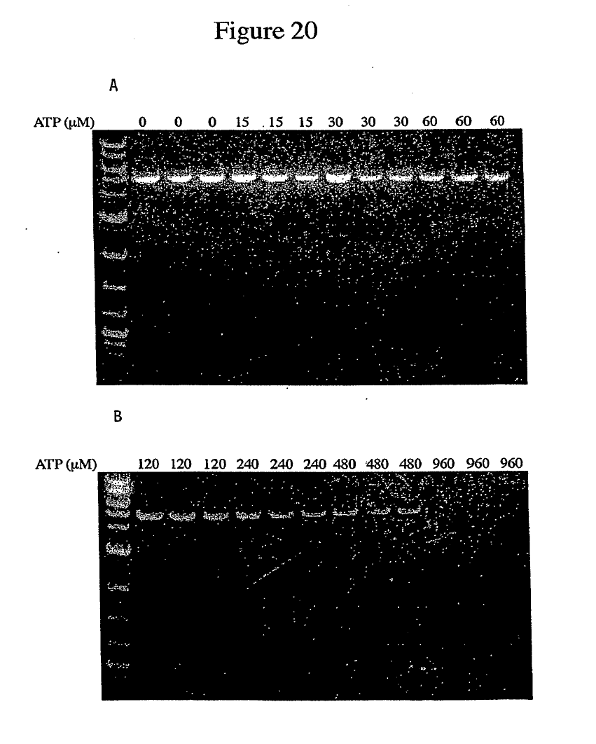

Embodiments of the invention are illustrated with Examples 1-20. These examples show that:
(I) Polynucleotide repair enhances amplicon yield (see Examples 1, 2, 3, 4, 5, 6, 7, 9, 13, 14, 17, 18 and 19). Associated with these examples, FIGS. 1B-1D and 4 show enhanced amplicon yield from heat-damaged lambda DNA after pre-incubation with a repair mixture; FIG. 2B shows enhanced amplicon yield from pH damaged DNA; FIG. 8C shows enhanced amplicon yield from UV-damaged DNA after treatment with various repair mixes. FIG. 14 shows enhanced amplicon yield from uracil containing plasmids after preincubation with a repair mixture. FIG. 15 shows increased yield of a 620 base pair DNA by repairing short oligonucleotides and amplifying the repaired DNA. FIG. 3 shows enhanced amplicon yield from krill DNA extracted from an ethanol sample after pre-treatment with a repair mixture; FIG. 5 shows enhanced amplicon yield from environmental DNA after pretreatment with a repair mixture; FIG. 17 shows increased amplicon yield of cave bear DNA. FIG. 19 shows the effect of pre-incubation of a repair mix on DNA samples that had been damaged by UV radiation, heat, oxidation, and by pH.
(II) Polynucleotide repair enhances fidelity of copies from a polynucleotide template (Examples 8, 9, 10, and 11). This is also illustrated in FIG. 11. FIG. 11 shows that cloning an amplicon of the lacZ gene results in a much lower percentage of white colonies if the damaged template is repaired prior to PCR. The lower the percentage of white colonies the greater the number of amplicons with the correct sequence.
(III) Polynucleotide repair facilitates down stream processing for example, for transformation, see Example 15, for cloning, see Example 16, and for sequencing, see Example 11; this is also illustrated in FIG. 16. FIG. 16 shows that more E. coli transformants are recovered from UV-damaged plasmid if it is first treated with a DNA repair mix.
(IV) Improved parameters for repair, such as incubation at a single temperature, see Example 12, a broad range of repair from a single mix, see Example 19, control of co-factor inhibition, see Example 20 and minimizing undesirable exonuclease affects, see Example 21. This is also illustrated in FIGS. 13, 19, and 20. FIG. 13 shows that incubation at a single temperature, either room temperature, 13A or 4° C., 13B, was able rescue amplification from UV-damaged DNA. FIG. 19 demonstrates that a single enzyme mix can repair a broad range of damages. FIG. 20 shows that an optimal ATP concentration can be found to minimize PCR inhibition by ATP.

All references cited herein, as well as U.S. application Ser. No. 11/255,290 filed Oct. 20, 2005 and U.S. provisional application Ser. Nos. 60/620,896 filed Oct. 21, 2004, 60/646,728 filed Jan. 24, 2005, 60/673,925 filed Apr. 21, 2005, and 60/791,056 filed Apr. 11, 2006, are incorporated by reference.

### EXAMPLES

### Example 1

### Enhancing Amplicon Yields from DNA Damaged by Heat Treatment

An assay was developed for optimizing the use of selected reagents to repair DNA prior to amplification.

### Generation of Various Extents of Heat Damage

Various amounts of DNA damage were induced by heat treatment. This was achieved as follows: 100 μL lambda DNA (NEB#N3011, NEB, Ipswich, Mass.) at 0.5 mg/ml was aliquoted into separate tubes for heat treatment at 99° C. for 30 sec, 60 sec, 90 sec, 120 sec, and 180 sec, respectively in a PE2700 thermal cycler. A sample was used as a template for amplification without pretreatment.

The remaining damaged DNA was pretreated by the mixture of enzymes as follows: The damaged DNA templates were incubated at room temperature in the following mixture for 10 minutes:

DNA (5 ng, 2 ng and 1 ng);

100 μM dNTPs (NEB#M0447, NEB, Ipswich, Mass.);

1 mM NAD⁺ (Sigma#N-7004, Sigma, St. Louis, Mo.);

80 units Taq DNA ligase (NEB#M0208, NEB, Ipswich, Mass.) or 40-100 units of E. coli DNA ligase (NEB#M0205S, NEB, Ipswich, Mass.)

0.1 units E. coli DNA polymerase I (E. coli pol I) NEB#M0209, NEB, Ipswich, Mass.);

10 units E. coli endonuclease IV (NEB#M0304, NEB, Ipswich, Mass.) or 10 units of Tth endonuclease IV;

1× Thermopol buffer (NEB#B9004, NEB, Ipswich, Mass.) to a final volume of 96 μL.

At the end of the reaction, the samples were transferred to ice and then amplified.

### DNA Amplification Reaction

DNA amplification of lambda was performed using the following primers: CGAACGTCGCGCAGAGAAACAGG (L72-5R) (SEQ ID NO:1) and CCTGCTCTGCCGCTTCACGC (L30350F) (SEQ ID NO:2) according to the method of Wang et al. Nucl. Acids Res. 32: 1197-1207 (2004).

4 μl of amplification mixture was added to 96 μl of the above repair mixture. The amplification mixture contained 100 μM dNTPs, 5 units Taq DNA polymerase, 0.1 units Vent® (exo+) DNA polymerase, 5×10⁻¹¹ M primer L72-5R and 5×10⁻¹¹ M primer L30350F in 1× Thermopol buffer.

To correct for any enzyme storage buffer effects, when a repair enzyme was omitted from a reaction, the appropriate volume of its storage buffer was added to the reaction. In all cases, the amplification reactions were processed in a thermal cycler using the following parameters: 20 sec at 95° C. for 1 cycle followed by 5 sec at 94° C., then 5 min at 72° C. for 25 cycles. The size of the amplicon being amplified was 5 kb.

The results of amplification of DNA (5 kb) were determined by 1% agarose gel electrophoresis. 6× loading dye (Molecular Cloning: A Laboratory Manual, 3^{rd} ed., eds. Sambrook and Russell, Cold Spring Harbor Press, Cold Spring Harbor, N.Y., 2001, pp. 5.4-5.17) was added to the 100 μl amplification reactions. 20 μl of this solution was then loaded onto the agarose gel along with 1 μg of 2-log ladder (NEB#N3200, NEB, Ipswich, Mass.) as a size standard.

The amount of amplified DNA for each sample was compared by gel electrophoresis and the results are shown in FIGS. 1A-D. When the samples were treated with a mixture of enzymes after heat treatment but prior to amplification, significant enhancement of amplification yields was achieved (FIGS. 1B, 1C and 1D).

### Example 2

### Enhanced Amplicon Yields from DNA with Low pH-Induced Abasic Sites Following Pretreatment with an Enzyme Mixture

Generation of Various Extents of Damage Resulting from Abasic Sites

To assay the extent of repair of damaged DNA, various amounts of DNA damage was first induced by acidic pH. This was achieved as follows:

DNA was depurinated as described by Ide, H., et al. Biochemistry 32(32):8276-83 (1993). Lambda DNA (NEB#N3011, NEB, Ipswich, Mass.) was ethanol precipitated. The DNA was resuspended in depurination buffer (100 mM NaCl, 10 mM citrate, pH 5.0) at a concentration of 0.5 mg/ml and incubated at 70° C. for 0, 20, 40, 80, 120, and 160 minutes. The sample was then ethanol precipitated and resuspended in 0.01 M Tris, 0.001 M EDTA, pH 8.0. The DNA concentration was determined by measuring the A₂₆₀ of the DNA-containing solutions after calibrating with a buffer control.

Pretreatment of DNA with a Mixture of Enzymes

The damaged DNA was incubated at room temperature for 10 minutes in the following mixture:

DNA (2.5 ng of damaged DNA after 120 minute of low pH treatment);

100 μM dNTPs;

1 mM NAD⁺;

80 units Taq DNA ligase;

0.1 units Taq DNA polymerase or 0.1 units E. coli PolI (NEB#M0209, NEB, Ipswich, Mass.)) or 0.1 units Taq: 0.002 units of Vent® Pol, (NEB#M0254, NEB, Ipswich, Mass.));

10 units E. coli endonuclease IV;

1× Thermopol buffer to a final volume of 96 μl.

The above mixture was incubated at room temperature for 10 minutes and then transferred to ice prior to amplification.

### DNA Amplification Reaction

Amplification was performed as described in Example 1 to generate a 5 kb amplicon. Amplicon yields were increased as compared with negative controls (FIG. 2A) by treating lambda DNA containing abasic sites with the mixture of enzymes. The results are shown in FIG. 2B for a series of pretreatments using different enzyme mixtures. The enzyme mixtures were varied with respect to the DNA polymerase (E. coli pol I, Taq Vent® pol I, Taq pol) in the presence of Taq DNA ligase.

### Example 3

### Enhanced Amplicon Yields of DNA Extracted from an Intact Organism after Storage in a Preservative

Genomic DNA was isolated from Meganyctiphanes norvegica (Krill) as described in Bucklin, A. & Allen, L. D. Mol. Phylogenet. Evol. 30(3):879-882 (2004). The Krill had been stored in ethanol for about 5 years.

Pretreatment of the Krill DNA by a mixture of enzymes was carried out as follows:

50 ng of M. norvegica genomic DNA;

100 μM dNTPs;

1 mM NAD⁺;

40 units of Taq DNA ligase;

0.5 units Taq DNA polymerase, 0.2 units Vent® (exo+) DNA polymerase, or a Taq:Vent® (exo+) mix containing 0.05 units of Taq DNA polymerase and 0.001 units of Vent® (exo+);

10 units E. coli endonuclease IV;

1× Thermopol buffer to a final volume of 96 μl.

This reaction was incubated 15 minutes at room temperature before proceeding to the amplification step.

### DNA Amplification Reaction

The amplification primers corresponded to 52F and 233R as described in Bucklin, A. & Allen, L. D. Mol. Phylogenet. Evol. 30(3):879-82 (2004) generating a 200 by amplicon.

| | |
|---|---|
| 52F: | |
| TTTTTAGCAATACACTACACAGCAA | (SEQ ID NO: 3) |
| 233R: | |
| ATTACGCCAATCGATCACG | (SEQ ID NO: 4) |

Primers were added to a final concentration of 0.5 μM, and each dNTP to a final concentration of 200 μM. 1 μl of the 50:1 Taq:Vent® mix (5 units Taq DNA polymerase and 0.1 units Vent® (exo+) DNA polymerase added to the reaction) was then added to each reaction to a final volume of 100 μL.

For the control reaction (Lane 1), no endonuclease IV, Taq DNA ligase or pretreatment DNA polymerase was added. Volumes were adjusted accordingly. In reactions in which repair enzymes were omitted, the appropriate volume of enzyme storage buffer was added to control for buffer effects.

Cycling conditions were as follows: 30 sec at 94° C., 30 sec at 52° C. and 1 min 40 sec at 72° C. for 40 cycles. 25 μl (one quarter of the reaction) was prepared, loaded on a 1% agarose gel, electrophoresed, and visualized as described above.

Increased amplicon yield from krill genomic DNA was observed after preincubation of the samples using the enzyme mixtures described above (FIG. 3).

### Example 4

### Enhanced Yields of a Large (10 Kb) Amplicon from Heat-Damaged DNA

Heat-damaged DNA was prepared as described in Example 1. Lambda DNA was heated to 99° C. for 180 sec.

Pretreatment of damaged DNA by a mixture of enzymes was carried out as follows:

Lambda DNA (1 μg of 180 sec heat-treated DNA);

100 μM dNTPs;

1 mM NAD⁺;

80 units of Taq DNA ligase;

0.1 unit of E. coli PolI;

100 units of E. coli endonuclease IV;

1× Thermopol buffer to a volume of 96 μL.

The mixture was incubated for 10 minutes prior to amplification.

DNA amplification was performed as described in Example 1, except where specified below. Primers were added to the above 96 of preincubation mixture. Primer L71-10R (sequence GCACAGAAGCTATTATGCGTCCCCAGG) (SEQ ID NO:5) replaced L72-5R in Example 1. The iCycler thermal cycler program (Bio-Rad, Hercules, Calif.) was: 20 sec at 95° C. for 1 cycle, 5 sec at 95° C., 10 min at 72° C. for 25 cycles and then 10 min at 72° C. for 1 cycle. Amplicon size was 10 kb.

The DNA was visualized as described in Example 1 with the following exceptions. 20 μl of 6× loading buffer was added to the 100 μl amplification reaction. 10 μl of this solution was diluted to 100 μl with H₂O and 1× loading buffer. 20 μl of this was loaded into each lane. The gel was a 0.8% agarose gel. The results are shown in FIG. 4.

### Example 5

### Improved Amplification Yield of DNA from Environmental DNA (Extracted from Soil Samples)

Environmental DNA was isolated from the soil using an UltraClean Soil DNA Kit from MoBio Laboratories, Inc., Carlsbad, Calif. (catalog #12800-50).

Pretreatment of DNA with a DNA Ligase

A final volume of 100 μl containing 0.6 μg of environmental DNA isolated from soil and one of the two DNA ligases described below in (a) and (b) formed the reaction mixture. This reaction mixture was then incubated at room temperature for 15 min.

(a) 1× Taq DNA ligase buffer (NEB, Ipswich, Mass.) and 80 units of Taq DNA ligase.

(b) 1× T4 DNA ligase buffer (NEB, Ipswich, Mass.) and 800 units of T4 DNA ligase (NEB#M0202, NEB, Ipswich, Mass.).

1 μl of reaction mixture was used in the amplification reaction described below.

DNA Amplification Reaction

DNA amplification was performed using primers:

| | |
|---|---|
| GGGGGXAGAGTTTGATCMTGGCTCA | (SEQ ID NO: 6) |
| and | |
| GGGGGXTACGGYTACCTTGTTACGACTT | (SEQ ID NO: 7) |

(M=C or A, Y=C or T, X=8-oxo-Guanine). These primers target 16S rDNA having an amplicon size of 1.6 Kb.

The 50 μl reaction contained 10 pmol of each of the primers, 1 μl of the repaired environmental DNA, 200 μM dNTPs, 1× Thermopol buffer, and 1.25 units Taq DNA polymerase. The amplification was performed using the following cycling parameters: 5 min at 94° C. for 1 cycle, 30 sec at 94° C., 1 min at 55° C., 1 min 40 sec at 72° C. for 32 cycles, then 5 min at 72° C. for 1 cycle.

Gel electrophoresis was performed as described in Example 1. The results are shown in FIG. 5.

### Example 6

### Enhanced Amplicon Yield of Ultraviolet Light-Damaged DNA

To determine conditions for assaying the effectiveness of DNA repair, 50 μg lambda DNA (NEB#N3011, NEB, Ipswich, Mass.) was diluted in TE buffer (10 mM Tris-HCl, 1 mM EDTA, pH 7.5) to a concentration of 50 μg/ml and irradiated with 36 J/m² UV light for 0, 10, 20, 30, 40 and 50 sec.

Pretreatment of damaged DNA by a mixture of enzymes was carried out as follows:

The damaged DNA was incubated at room temperature for 15 minutes in the following mixture:

DNA (50 ng of lambda DNA-damaged for 0, 10, 20, 30, 40, or 50 seconds);

200 μM dNTPs;

1 mM NAD⁺;

400 units Taq DNA ligase;

0.1 units E. coli DNA polymerase I;

10 units E. coli endonuclease IV;

80 units or 10 units T4 PDG (also referred to as T4 endonuclease V); (Trevigen, Gaithersburg, Md.);

1× Thermopol buffer

Adjust volume with water to 50 μl.

After the 15 minutes incubation, the 50 μl reaction mixture was added to 50 μl of an amplification solution. The amplification solution consisted of 40 pmol of each primer (L72-5R and L30350F as described in Example 1 or L72-2R (the DNA sequence was CCATGATTCAGTGTGCCCGTCTGG) (SEQ ID NO:8), 1× Thermopol buffer, 1 mM NAD⁺, 200 μM dNTPs, 2.5 units Taq DNA polymerase (NEB#M0267, NEB, Ipswich, Mass.), and H₂O to a final volume of 50 μL. Combining the 50 μL repair reaction with the 50 μl amplification solution gave a final volume of 100 μl.

The 100 μl solutions were placed into a thermal cycler.

For the L72-5R and L30350F primer combination:
5 min at 94° C. for 1 cycle; 30 sec at 94° C., 60 sec at 58° C., and 4 min at 72° C. for 30 cycles; 5 min at 72° C. for 1 cycle.
For the L72-2R and L30350F primer combination:
5 min at 94° C. for 1 cycle; 30 sec at 94° C., 60 sec at 58° C., and 2 min at 72° C. for 30 cycles; 5 min at 72° C. for 1 cycle.

The presence of amplification product was visualized on a 1.8% agarose gel using ethidium bromide. The size of any band was compared against a lane containing the 2-log ladder (NEB#N3200S, NEB, Ipswich, Mass.) size standards. The results are shown in FIG. 8.

### Example 7

### Enhanced Amplicon Yield of DNA Using the Nucleotide Excision Repair Proteins, UvrA, UvrB and UvrC

Increased amplicon yield from krill genomic DNA is determined after pre-incubation of the samples using an enzyme mixture containing proteins involved in nucleotide excision repair.

Pretreatment of stored DNA by a mixture of enzymes is carried out as follows:

Stored DNA is incubated for 1-180 minutes at 4-37° C. in the following mixture:

DNA: 50 ng of M. norvegica genomic DNA;

100 μM dNTPs;

1 mM ATP;

400 units of Taq DNA ligase;

0.1 units E. coli DNA polymerase I;

10 nM E. coli UvrA, 250 nM E. coli UvrB (or mutant UvrB*), plus or minus 50 nM E. coli UvrC;

1× Thermopol buffer to a final volume of 96 μl.

* for mutant UvrB, see Zou, Y., et al. Biochemistry 43:4196-4205 (2004).

DNA amplification reactions are conducted as described in Example 3.

### Example 8

### Increasing Sequence Accuracy of a DNA after Removal of Incorrect Nucleotides on at Least One Strand by Means of Enzyme Cleavage of Heteroduplexes

A. Adding Taq DNA ligase to T7 endonuclease I permitted the use of an increased concentration of T7 endonuclease I in a DNA preparation without randomly degrading the DNA.

The assay relied on treating a supercoiled DNA containing a cruciform structure with increasing amounts of T7 endonuclease I.

0, 1.6, 3.1, 6.2, 12.5, 25, 50, 100, 200, or 400 units of T7 endonuclease I (NEB#M0302, NEB, Ipswich, Mass.) was added to 50 μl reactions composed of 1 μg of pUC(AT) (Guan, C., et. al. Biochemistry 43:4313-4322 (2004)) and 1× NEBuffer 2 (NEB#B7002S, NEB, Ipswich, Mass.). Plasmid pAT25tetA can be used in place of pUC(AT) (Parkinson, M. J. & Lilley, D. M. J. Mol. Biol. 270:169-178 (1997) and Bowater, R. P., et. al. Biochemistry 33:9266-9275 (1994)). Another set of reactions were set up simultaneously and used the same components as described above with the addition of 1 mM NAD⁺ (Sigma catalog#N-7004, Sigma, St. Louis, Mo.) and 100 units of Taq DNA ligase (using a stock of NEB#M0208 at a concentration of 100 u/μl, NEB, Ipswich, Mass.). All reactions were incubated at 37° C. for 60 minutes.

The results were analyzed by running the reactions on a 0.9% TBE agarose gel, stained with ethidium bromide, and visualized using UV light (see FIGS. 9A and 9B). With no T7 endonuclease I present, the pUC(AT) plasmid produced 2 bands on the gel corresponding to the supercoiled form (lower band) and the relaxed circular form (upper band).

T7 endonuclease I resolved the supercoiled pUC(AT) into the relaxed circular form and a linear form that ran intermediate to the supercoiled and relaxed circular forms. At certain T7 endonuclease I to DNA ratios, a smear was produced indicating that the T7 endonuclease I had degraded the DNA by non-specific enzymatic activity. The presence of Taq DNA ligase significantly increased the usable T7 endonuclease I to DNA ratio. This ratio is further improved by substituting T7 endonuclease I with the mutant T7 endonuclease I described in International Publication No. WO 2005/052124.

B. The use of Method A to remove heterduplexes from PCR reactions.

Isolation of DNA from soil and amplification of the purified DNA is performed as described in Example 5 with the optional addition of 5 units T7 endonuclease I or mutant thereof. When T7 endonuclease I or mutant thereof is added, an additional amplification cycle is added (37° C. for 15 minutes for 1 cycle). The last step is to allow the T7 endonuclease I to cleave any heteroduplexes formed.

Gel electrophoresis is performed as described in Example 1. Heteroduplex DNA is visualized on the gel as described in Lowell, J. L. & Klein, D. A. Biotechniques 28:676-681 (2000). Absence of heteroduplex DNA in the presence of T7 endonuclease I or mutants thereof shows the effectiveness of T7 endonuclease I or mutants thereof with a DNA ligase.

Unit definitions are described with the product description for each of the enzymes recited herein in the NEB catalog, NEB, Ipswich, Mass. For example, the unit definition for T7 endonuclease I or mutant thereof is the amount of enzyme required to convert greater than 90% of 1 μg of supercoiled plasmid into greater than 90% linear DNA in a reaction volume of 50 μl in 1 hour at 37° C.

The T7 endonuclease I to DNA ratio can be increased without increasing non-specific cleavage of DNA in the presence of DNA ligase.

### Example 9

### Enhancing the Sequence Accuracy of a DNA Amplification Reaction after Oxidative Damage

Generating DNA with Oxidative Damage

The pWB407 DNA (Kermekchiev, M. B. et al. Nucl. Acids Res. 31:6139-47 (2003)) was subjected to oxidative damage. The damage was incurred using a combination of methylene blue (MB) and visible light as described previously (Sattler, et al. Arch. Biochem Biophys. 376(1):26-3 (2000)). Plasmid DNA (200 μg/ml in distilled water) was spotted on parafilm stretches (50 μl drops). MB was added to the drops to a final concentration ranging from 0 to 50 (0, 3, 6, 12.5, 25 and 50) μg/ml (100 μl final volume). Plates with these parafilm stretches were placed on ice and illuminated for 8 min. with a 1×100-W lamp. The MB-light-treated DNA was precipitated, dried, and then re-suspended in 50 μl of TE buffer (pH 8.0). Final DNA concentration was determined by the absorbance of light at 260 nm.

### DNA Amplification Conditions

A portion of pWB407 that contained the lacZ gene was amplified using primers 316-138, TGTCGATCAGGATGATCTGGACGAAGAGC (SEQ ID NO:9), and 316-137, CGAAAGCTTTCAAGGATCTTACCGCTGTTGAGA (SEQ ID NO:10). Primers 316-138 and 316-137 were based on the previously-described primers Kfd-29 and H3Bla34, respectively (Kermekchiev, M. B. et al. Nucl. Acids Res. 31:6139-47 (2003)). The 100 μL PCR reactions contained either 10 or 50 ng of template DNA, indicated where appropriate, and 40 picomoles of each primer. The cycling conditions utilized varied with the thermal stability of the DNA polymerase used for amplification.

Cycling conditions when using Taq DNA polymerase (NEB cat#M0267S, NEB, Ipswich, Mass.) were an initial denaturation step of 5 min at 94° C. for 1 cycle, then 30 sec at 94° C., 60 sec at 58° C., and 3 min 30 sec at 72° C. for 30 cycles, and finally 5 minutes at 72° C.

Cycling conditions when using Phusion™ DNA polymerase (NEB cat#F-530S, NEB, Ipswich, Mass.) were an initial denaturation step of 30 sec at 98° C. for 1 cycle, then 10 sec at 98° C., 30 sec at 62° C., and 1 min 30 sec at 72° C. for 30 cycles, and finally 5 min at 72° C.

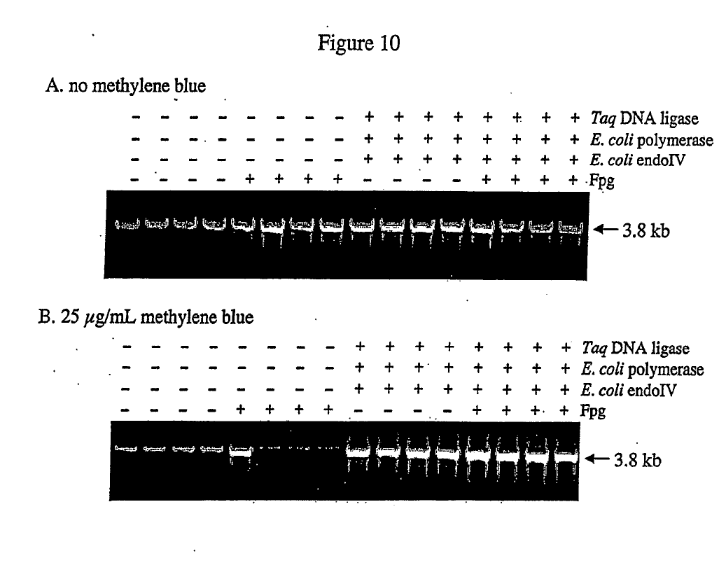

The reaction outcomes were analyzed by loading 25 μL of the reaction mixture on a 1.6% agarose gel, prepared, electrophoresed and visualized as described above (FIG. 10). The marker used was the 2-log DNA ladder (NEB cat#N3200S, NEB, Ipswich, Mass.).

### Amplification Accuracy Determination

The accuracy of DNA amplification from the pWB407 template was determined as described by Barnes, et al. Gene 112:29-35 (1992) and Kermekchiev, et al. Nucl. Acids Res. 31:6139-47 (2003). Amplicons containing the lacZ gene were generated from plasmids pWB407 that had been subjected to differing amounts of oxidative damage. The oxidative damage was performed using methylene blue as described above. The PCR reactions were performed using 50 ng of template as described above. After cycling, 10 units of the restriction endonuclease, DpnI (NEB, Ipswich, Mass.), was added to each 100 μL PCR reaction and incubated for 2 hours at 37° C. This step eliminated the original template plasmid. Next, the resulting amplification products were extracted with phenol/chloroform and precipitated using isopropanol (Molecular Cloning: A Laboratory Manual, 3^{rd} ed., eds. Sambrook and Russell, Cold Spring Harbor Press, Cold Spring Harbor, N.Y., 2001, pp. 6.25, A8.12-A8.24). Precipitated products were re-suspended in H₂O and cut with the restriction endonucleases StyI and HindIII using conditions recommended by the manufacturer (NEB, Ipswich, Mass.). The DNA digestion reactions were stopped by inactivating the HindIII and StyI enzymes by heating to 65° C. for 20 min. The restriction digestion products were purified using a microcon YM-100 column (Millipore, Billerica, Mass.) to eliminate short DNA fragments.

The repair reaction mixtures in a total of 50 μl contained 10 or 50 ng of pWB407 amplicons +/− methylene blue incubation. The repair reactions contained 20 mM Tris-HCl (pH 8.8 at 25° C.), 10 mM KCl, 10 mM (NH₄)₂SO₄, 2 mM MgSO₄, 0.1% Triton X-100, 1 mM NAD⁺, 200 μM dNTPs (dATP, dTTP, dCTP, and dGTP), and various repair enzyme mixtures.

The repair enzyme mixtures used separately or in various combinations in a total volume of 50 μL were:

0.4 units Fpg, NEB cat#M0240S, NEB, Ipswich, Mass.);

200 units Taq DNA ligase;

0.1 units E. coli DNA polymerase I;

10 units E. coli endonuclease IV;

1 mM NAD⁺;

100 μM dNTPs;

1× Thermopol buffer.

The reactions were incubated at 25° C. for 15 minutes. After the incubation, 50 μL of a PCR mix (20 mM Tris-HCl (pH 8.8 at 25° C.), 10 mM KCl, 10 mM (NH₄)₂SO₄, 2 mM MgSO₄, 0.1% Triton X-100, 1 mM NAD⁺, 200 μM dNTPs (dATP, dTTP, dCTP, and dGTP), and either 2.5 units Taq DNA polymerase (NEB cat#M0267S, NEB, Ipswich, Mass.) or 1 unit of Phusion™ DNA polymerase was added to the 50 μL repair reaction and this new solution was subjected to thermocycling conditions for PCR. The amplicons from these reactions were purified and restriction enzyme digested as described for other amplicons above.

The amplicons were cloned into the pWB407 plasmid. Plasmid pWB407 was prepared by digestion with the restriction endonucleases StyI and HindIII followed by a 30-minute incubation at 37° C. with 1 unit/μg DNA of antarctic phosphatase (NEB cat#M0289S, NEB, Ipswich, Mass.). The dephosphorylated pWB407 vector backbone was purified by agarose gel electrophoresis. Gel extraction was performed with a QIAquick Gel Extraction Kit (Qiagen, Valencia, Calif.).

The digested amplicons were ligated into the prepared pWB407 plasmids in 30 μL reactions using approximately 0.1 μg vector DNA and about 0.5 μg amplicon. T4 DNA ligase was used to perform the ligation following the manufacturers recommended conditions (NEB, Ipswich, Mass.). Ligation products were electroporated into E. coli strain WB441 (Barnes, W. Gene 112:29-35 (1992)). The selective indicator plates used were LB plates containing 50 μg/ml ampicilin and 80 μg/ml Xgal. Before plating, the bacteria were incubated in rich broth for 1 hour at 37° C. to allow expression of the ampicilin resistance. Control transformations lacking DNA ligase treatment resulted in zero colonies. Colonies were scored for blue color after one day at 37° C., and one or two days at 25° C. The results are shown in FIGS. 10A-10B and 11.

### Example 10

### Enhancing the Sequence Accuracy of a DNA Amplification Reaction after Deamination Damage

### Generating Deaminated DNA

The DNA subjected to deamination is pWB407 (Kermekchiev, et al. Nucl Acids Res 31: 6139-6147 (2003)). The damage is incurred using random mutagenesis with nitrous acid as described in Van, W. et al. J. Virol. 77(4):2640-50 (2003). Nitrous acid can deaminate guanine in DNA to xanthine, cytosine to uracil, and adenine to hypoxanthine.

Plasmid DNA (2 μg) is treated with 0.7 M NaNO₂ in 1M acetate buffer, pH 4.6. The reaction is terminated at various time points by addition of 4 volumes of ice-cold 1 M Tris-HCl (pH 7.9). The plasmid DNA is alcohol precipitated, dried and then resuspended in 100 μL of TE buffer.

### Pretreatment Reaction to Repair Deaminated Bases

The repair enzyme mixtures used separately or in various combinations in a total volume of 50 μL are:

(a)

1 unit Human Aag;

2 units endonuclease (NEB cat #M0268S), NEB, Ipswich, Mass.;

2 units endonuclease V (NEB cat #M03055), NEB, Ipswich, Mass.;

2 units UDG (NEB cat #M0280S), NEB, Ipswich, Mass.;

200 units Taq DNA ligase;

0.1 units E. coli DNA polymerase I;

10 units E. coli endonuclease IV;

1 mM NAD⁺;

100 μM dNTPs;

1× Thermopol buffer.

(b)

2 units endonuclease V (NEB cat #M0305S), NEB, Ipswich, Mass.;

2 units UDG (NEB cat #M02805), NEB, Ipswich, Mass.;

200 units Taq DNA ligase;

0.1 units E. coli DNA polymerase I;

10 units E. coli endonuclease IV;

1 mM NAD⁺;

100 μM dNTPs;

1× Thermopol buffer.

(c)

2 units endonuclease V (NEB cat #M0305S), NEB., Ipswich, Mass.;

200 units Taq DNA ligase;

0.1 units E. coli DNA polymerase I;

10 units E. coli endonuclease IV;

1 mM NAD⁺;

100 μM dNTPs;

1× Thermopol buffer.

(d)

1 unit Human Aag, NEB, Ipswich, Mass.;

2 units endonuclease III (NEB cat #M0268S), NEB, Ipswich, Mass.;

200 units Taq DNA ligase;

0.1 units E. coli DNA polymerase I;

10 units E. coli endonuclease IV;

1 mM NAD⁺;

100 μM dNTPs;

1× Thermopol buffer.

(e)

1 unit Human Aag, NEB, Ipswich, Mass.;

2 units UDG (NEB cat #M02805), NEB, Ipswich, Mass.;

200 units Taq DNA ligase;

0.1 units E. coli DNA polymerase I;

10 units E. coli endonuclease IV;

1 mM NAD⁺;

100 μM dNTPs;

1× Thermopol buffer.

(f)

1 unit Human Aag, NEB, Ipswich, Mass.;

2 units endonuclease V (NEB cat #M03055), NEB, Ipswich, Mass.;

200 units Taq DNA ligase;

0.1 unit E. coli DNA polymerase I;

10 units E. coli endonuclease IV;

1 mM NAD⁺;

100 μM dNTPs;

1× Thermopol buffer.

The amplification reaction conditions and amplification accuracy determination are performed as described in Example 9.

### Example 11

### Repair of DNA Prior to Use in DNA Sequencinq Reactions to Increase the Sensitivity of the Sequencinq Reactions

The sensitivity of the sequencing reaction is intended to mean that the amount of template DNA having a correct sequence prior to sequencing results in reduced background noise and increased signal. This makes possible longer and/or more complete sequence reads. The improved fidelity of the sequence read is an additional benefit. The beneficial use of a repair mix such as described below can be observed for sequencing methods in general. For example, sequencing methods include 454 sequencing, single molecule sequencing, Sanger sequencing and Maxam-Gilbert sequencing.

Two DNA samples are subjected to DNA sequencing before and after DNA repair. The two DNA samples are UV-treated for 40 seconds (see Example 6) and lambda DNA is exposed to light in the presence of 25 μg/mL methylene blue.

Prior to use in the DNA sequencing reaction the DNA to be sequenced is contacted with one or more repair enzymes under conditions that permit activity of the repair enzymes. For example, 0.5 μg template DNA for sequencing is mixed with NEB Thermopol buffer to 1× concentration (NEB, Ipswich, Mass.) (1× concentration of Thermopol buffer contains 20 mM Tris-HCl, pH 8.8 at 25° C., 10 mM KCl, 10 mM (NH₄)₂SO₄, 2 mM MgSO₄, 0.1% Triton X-100) and incubated for 15 minutes at room temperature with a DNA repair mixture (200 units Taq DNA ligase, 0.1 units E. coli pol I, 1 unit T4 PDG, 15 units endonuclease IV, 2.5 units endonuclease VIII, 0.1 unit Fpg, and optionally 0.5 unit E. coli UDG) in a volume of 100 μL. The repaired DNA is either used immediately for sequencing or is purified and concentrated using a commercial kit (a Qiagen, Inc., Valencia, Calif. kit, for example) prior to DNA sequencing. The sequencing reaction may be performed by the classical Sanger sequencing reactions or by methods described in U.S. Publication No. 2005/0100932, U.S. Pat. No. 6,897,023, or Margulies, et al. Nature 437(7057):376-80 (2005).

The sensitivity of the sequencing reaction and fidelity of the results are improved as a result of the pre-incubation with the repair mixture.

### Example 12

### A Multi-Enzyme Repair Mix for Repairing Damaged DNA is Effective at a Single Temperature

Lambda DNA was treated by 30s irradiation with UV (see Example 6). L72-5R (SEQ ID NO:1) and L30350F (SEQ ID NO:2) primers were selected for amplifying a 5 kilobase amplicon from the UV-treated lambda DNA either with or without prior repair. The DNA repair mix contained 200 units/μL Taq DNA ligase, 0.1 units/μL E. coli pol I, 1 unit/μL T4 PDG, 15 units/μL endonuclease IV, 0.5 unit/μL E. coli UDG, 2.5 units/μL endonuclease VIII, and 0.1 unit/μL Fpg stored in 20 mM Tris-HCl, pH 7.5 at 25° C., 100 mM NaCl, and 50% glycerol. Fifty ng of the 30 s UV-treated lambda DNA was added to thermocycler tubes each containing, 1× Thermopol buffer, 100 μM dNTPs, and 0.5 mM NAD⁺. 1 μL of the repair enzyme mix was added to 4 out of the 8 tubes and all were brought to a final volume of 47 μL with H₂O. Two tubes containing repair enzymes and 2 tubes lacking the enzymes were incubated at room temperature for 15 minutes. The remaining solutions were incubated overnight at 4° C. After the indicated incubation times, the primers (1 μM), dNTPs (100 μM), and 2.5 units Taq DNA polymerase were added to each thermocycler tube and the solutions placed into a Mycycler thermocycler running the program (Bio-Rad, Hercules, Calif.): 95° C. for 2 min, one cycle; 95° C. for 10 sec, 60° C. for 30 sec and 72° C. for 5 min, 25 cycles; 72° C. for 5 min, one cycle; and a 4° C. hold. 25 μL of each reaction was analyzed on a 1% agarose gel.

In contrast to the findings of others (U.S. Publication No. 2006/0014154), which required the use of multiple different temperatures to achieve repair, incubation of UV-damaged DNA with the above repair mixture at room temperature for 15 minutes or 4° C. overnight produced an amplification product of the correct size (FIGS. 13A and 13B).

### Example 13

### Repair of Plasmid DNA Containing Multiple Uracils and Amplification Using Vent® DNA Polymerase

Plasmid pNEB0.92U was purified from E. coli CJ236 (NEB# E4141S, NEB, Ipswich, Mass.). The sequence is shown in FIG. 18. Because E. coli CJ236 lacks dUTPase and uracil-N-glycosylase, this plasmid contains uracils randomly distributed throughout its sequence. The archaeal DNA polymerase Vent® DNA polymerase is inhibited by uracil containing templates. Amplification of a 920 base amplicon from pNEB0.92U DNA was examined using primers S1224S (CGCCAGGGTTTTCCCAGTCACGAC) (SEQ ID NO:12 and S1233S (AGCGGATAACAATTTCACACAGGA) (SEQ ID NO:13) either with or without prior repair. The DNA repair mix contained 200 units/μL Taq DNA ligase, 0.1 units/μL E. coli PolI, 1 unit/μL T4 PDG, 15 units/μL endonuclease IV, 0.5 unit/μL E. coli UDG, 2.5 units/μL endonuclease VIII, and 0.1 unit/μL Fpg and stored in 20 mM Tris-HCl, pH 7.5 at 25° C., 100 mM NaCl, and 50% glycerol. One μL of the repair enzyme mix was added to 2 of 4 thermocycler tubes each containing 0.5 ng pNEB0.92U, 1× Thermopol buffer, 100 μM dNTPs, and 0.5 mM NAD⁺. All were brought to a final volume of 45 μL with H₂O. The reaction solutions were incubated at room temperature for 15 minutes after which the primers (final concentration: 0.4 μM), dNTPs (final concentration: 100 μM), and 1 unit Vent® DNA polymerase were added to each tube. The solutions were placed into a Mycycler thermocycler running the program: 95° C. for 2 min, one cycle; 95° C. for 10 sec, 65° C. for 30 sec and 72° C. for 1 min, 25 cycles; 72° C. for 5 min, one cycle; then a 4° C. hold. 25 μL of each reaction was examined by electrophoresis on a 1% agarose gel.

Amplification from pNEB0.92U without removal of uracils using Vent® DNA polymerase produced a barely detectable product of the desired size. Treatment of the pNEB0.92U with the repair mix significantly increased the amount of amplicon produced from this plasmid using Vent® DNA polymerase (FIG. 14).

### Example 14

### Enhanced Amplicon Yield from DNA Fragments

The template in this reaction was a set of 20 overlapping synthetic single strand oligonucleotides with an average size of approximately 45 nucleotides. The oligonucleotide sequences are shown below:

NEB oligo No. 316-219 (NEB, Ipswich, MA):

(SEQ ID NO: 14)

GGCGGCCTCGAGGCGAAACGCCGCAACTGCTTTCCGGGCGATACC
NEB oligo No. 316-220 (NEB, Ipswich, MA):

(SEQ ID NO: 15)

CGGCACGCCATCAATCTGCACCAGAATGCGGGTATCGCCCGGAAA
NEB oligo No. 316-221 (NEB, Ipswich, MA):

(SEQ ID NO: 16)

ATTGATGGCGTGCCGCAGAAAATTACCCTGCGCGAACTGTATGAA
NEB oligo No. 316-222 (NEB, Ipswich, MA):

(SEQ ID NO: 17)

CATGTTTTCATAGCGTTCATCTTCAAACAGTTCATACAGTTCGCG
NEB oligo No. 316-223 (NEB, Ipswich, MA):

(SEQ ID NO: 18)

CGCTATGAAAACATGGTGTATGTGCGCAAAAAACCGAAACGCGAA
NEB oligo No. 316-224 (NEB, Ipswich, MA):

(SEQ ID NO: 19)

GGTTTCCAGGTCAATGCTATACACTTTAATTTCGCGTTTCGGTTT
NEB oligo No. 316-227 (NEB, Ipswich, MA):

(SEQ ID NO: 20)

ATTGACCTGGAAACCGGCAAAGTGGTGCTGACCGATATTGAAGAT
NEB oligo No. 316-228 (NEB, Ipswich, MA):

(SEQ ID NO: 21)

CAGATGATCGGTCGCCGGCGCTTTAATCACATCTTCAATATCGGT
NEB oligo No. 316-229 (NEB, Ipswich, MA):

(SEQ ID NO: 22)

GCGACCGATCATCTGATTCGCTTTGAACTGGAAGATGGCCGCAGC
NEB oligo No. 316-230 (NEB, Ipswich, MA):

(SEQ ID NO: 23)

CAGCACCGGATGATCCACGGTGGTTTCAAAGCTGCGGCCATCTTC
NEB oligo No. 316-233 (NEB, Ipswich, MA):

(SEQ ID NO: 24)

GATCATCCGGTGCTGGTGTATGAAAACGGCCGCTTTATTGAAAAA
NEB oligo No. 316-234 (NEB, Ipswich, MA):

(SEQ ID NO: 25)

TTTATCGCCTTCTTTCACTTCAAACGCGCGTTTTTCAATAAAGCG
NEB oligo No. 316-237 (NEB, Ipswich, MA):

(SEQ ID NO: 26)

AAAGAAGGCGATAAAGTGCTGGTGAGCGAACTGGAACTGGTGGAA
NEB oligo No. 316-238 (NEB, Ipswich, MA):

(SEQ ID NO: 27)

TTTCGGGTTATCCTGGCTGCTGCTGCTCTGTTCCACCAGTTCCAG
NEB oligo No. 316-239 (NEB, Ipswich, MA):

(SEQ ID NO: 28)

CAGGATAACCCGAAAAACGAAAACCTGGGCAGCCCGGAACATGAT
NEB oligo No. 316-240 (NEB, Ipswich, MA):

(SEQ ID NO: 29)

ATATTTAATGTTTTTAATTTCCAGCAGCTGATCATGTTCCGGGCT
NEB oligo No. 316-247 (NEB, Ipswich, MA):

(SEQ ID NO: 30)

AAAAACATTAAATATGTGCGCGCGAACGATGATTTTGTGTTTAGCCTG
NEB oligo No. 316-248 (NEB, Ipswich, MA):

(SEQ ID NO: 31)

TTAATAATCACGTTATGATATTTTTTCGCGTTCAGGCTAAACACAAA
NEB oligo No. 316-265 (NEB, Ipswich, MA):

(SEQ ID NO: 32)

TAACGTGATTATTAACGAAAACATTGTGACCCATGCGTGCGATG
NEB oligo No. 316-266 (NEB, Ipswich, MA):

(SEQ ID NO: 33)

GCCGCCCTGCAGACCGGTCAGATCTTCATCGCCATCGCACGCATGGG

The assembly reaction consisted of two parts: an assembly step and an amplification step. For the assembly step the standard reaction was 50 μL and contained 70 nM of each oligo, 100 μM dNTP, 0.5 mM NAD⁺, 10 mM Tris-HCl, pH 7.5 at 25° C., 2 mM MgCl₂, and 50 mM NaCl. No enzymes were added to the control reaction. The first experimental reaction also contained 400 units Taq DNA ligase, 0.1 units E. coli pol I, 5 units T4 PDG, and 20 units endonuclease IV. The second set of reactions contained enzymes used in the first reaction and lambda beta protein (Kmiec, et al. J. Biol. Chem. 256:12636-12639 (1981); Rybalchenko, N., et al, Proc. Natl. Acad. Sci. USA 101:17056-17060 (2004)) added at a 1:1 beta protein:nucleotide mole ratio. The third reaction set contained 400 units Taq DNA ligase, 0.1 units E. coli pol I, 5 units T4 PDG, 20 units endonuclease IV, 0.3 mM ATP, and a 3:1 nucleotide to RecA mole ratio. The RecA was from E. coli (NEB catalog #M0249L, NEB, Ipswich, Mass.). The fourth reaction contained 400 units Taq DNA ligase, 0.1 units E. coli pol I, 5 units T4 PDG, 20 units endonuclease IV, 0.3 mM ATP, a 3:1 nucleotide to RecA mole ratio, and a 1:1 beta protein to nucleotide mole ratio. Glycerol content in each reaction was controlled for. The reaction mixtures were incubated for 30 minutes at room temperature.

After room temperature incubation, 5 μL of the assembly reaction was amplified using 200 μM dNTPs, 500 nM in oligonucleotides 316-219 and 316-266, 6 mM in MgSO₄, 1 unit of Vent® DNA Polymerase, and 1× Thermopol buffer final volume of 100 μL. The reactions were mixed and placed in a Mycycler (Bio-Rad, Hercules, Calif.) and the following thermal cycler touchdown program was used: 94° C. for 2 minutes (1 cycle); 94° C. for 30 seconds, 72° C.-62° C. (decreasing 1° C. per cycle) for 30 seconds, 72° C. for 45 seconds (10 cycles); 94° C. for 30 seconds, 62° C. for 30 seconds, 72° C. for 45 seconds (20 cycles); 72° C. for 5 minutes (1 cycle), and a 4° C. hold. Each reaction was performed in duplicate. 11 μL of 10× sample buffer was added to each sample and 25 μL was loaded onto a 1% agarose gel for electrophoresis.

The results are shown in FIG. 15. When the assembly reaction contained no added repair proteins, no amplification product was detected after the amplification step. However, when repair proteins were added in the assembly reactions, the correct 620 by amplicon was obtained from the amplification step. Inclusion of lambda beta protein and/or E. coli RecA further increased the yield of amplicon. It was concluded that in a system in which a DNA template is composed of fragments, the inclusion of DNA repair proteins facilitates the ability to produce an amplicon. Furthermore, this effect is enhanced when some of those DNA repair proteins are known to be involved in DNA recombination.

### Example 15

### Enhanced Transformation Efficiency with Damaged Plasmid DNA for E. coli

The plasmid pUC19 (GenBank Accession #L09137) was applied to a 1% agarose gel and electrophoresed in the presence of ethidium bromide until the plasmid had moved into the gel. The DNA in the gel was subjected to 254 nm UV light for 60 seconds. After the UV exposure a gel plug containing the pUC19 plasmid was excised. The plasmid was extracted from the gel plug using a Qiagen gel extraction kit (Qiagen, Valencia, Calif.). 30 ng of UV-irradiated DNA or non-irradiated DNA in a final volume of 25 μL was treated with 50 units E. coli DNA ligase (NEB#M0205S, NEB, Ipswich, Mass.), 0.1 units E. coli PolI, 5 units T4 PDG, and 20 units endonuclease IV in a buffer of 1× Thermopol buffer (NEB#B9004S, NEB, Ipswich, Mass.) with added NAD⁺ (Sigma product #N-7004, Sigma-Aldrich, St. Louis, Mo.) and dNTPs (NEB#NO447S, NEB, Ipswich, Mass.) to 0.5 mM and 100 μM, respectively. The reaction was incubated 15 minutes at room temperature before using the DNA to transform E. coli DH-5 alpha (NEB#C2991H, NEB, Ipswich, Mass.). As a control, both UV-irradiated and non-irradiated DNA were treated as above in the absence of added enzymes. The DH5 alpha cells were transformed with UV-irradiated and non-irradiated plasmid DNA that had been treated with repair enzymes or not so treated. The transformation was performed by heat shocking the E. coli in the presence of plasmid DNA. 50 μL of E. coli and plasmid were incubated on ice for 30 minutes before a 30 second incubation at 42° C. The transformation reaction was then placed on ice for 2 minutes before plating the cells on LB agar plates containing 100 μg/mL ampicillin. LB agar plates with differing dilutions of each transformation were placed in a 37° C. incubator overnight to determine the transformation efficiency of the plasmid.

Plasmid pUC19 that had been subjected to UV-irradiation and not repaired had a significantly reduced transformation efficiency when compared to undamaged pUC19 plasmid that had been treated with a repair enzyme mix (see FIG. 16).

### Example 16

### Simultaneous Repair and Blunting of DNA for Subsequent Ligation Required for PCR, Cloning or Immobilization

DNA libraries are commonly made from the environment, tissues, or cell cultures (Brady, S. F., et al. Applied and Environmental Microbiology, 70(11):6865-6870 (2004); Current Protocols in Molecular Biology, Vol. 1, Ausubel, F., et al (editors), John Wiley & Sons, Inc., Hoboken, N.J.; Chapter 5: “Construction of Recombinant DNA Libraries” (2004); Courtois, S., et al., Applied and Environmental Microbiology, 69(1):49-55 (2003); U.S. Pat. No. 6,444,426). These libraries are routinely created by shearing the DNA from the desired source by sonication, enzymatic treatment, or nebulization, preparing the DNA ends and ligating the mixture to oligonucleotides or plasmid DNA (Weinmann, A. S., Molecular and Cellular Biology, 21(20):6820-6832 (2001)). Ligation to oligonucleotides permits subsequent PCR or immobilization on arrays containing DNA sequences complimentary to the ligated oligo. Ligation to a plasmid permits the propagation in a heterologous host. A recent use of libraries is in chromatin immunoprecipitation (Guenther, M. G., et al. Proc. Natl. Acad. Sci. USA 102(24):8603-8608 (2005); Ren, B., et al. Genes Dev. 16:245-256 (2002); and Odom, D. T., et al. Science 303:1378-1381 (2004)). As part of preparing DNA ends for blunt end ligation, researchers often use a DNA polymerase such as T4 DNA polymerase. However, an enzyme mix is provided here that can not only repair damage that the DNA may have acquired during purification, preparation and storage, but can also create blunt ends. This enzyme mix includes a DNA ligase, an effective amount of an AP endonuclease, a proof-reading DNA polymerase and any cofactors necessary to allow enzyme activity. Preferably, the mix is composed of a DNA ligase, a proof-reading DNA polymerase, an apurinic/apyrimidinic endonuclease, UDG, FPG, and T4 PDG.

As an example, Chromatin IP (ChIP) is performed on HeLa cell DNA using antibodies to E2F1, E2F2, E2F3, E2F4, E2F5, or E2F6 as described previously (Weinmann, A. S. Molecular and Cellular Biology 21(20):6820-6832 (2001)). The cloning of ChIP enriched DNA is as described previously (Weinmann, A. S. Molecular and Cellular Biology 21(20):6820-6832 (2001)). The use of T4 DNA polymerase alone to blunt the DNA is replaced by an enzyme mixture containing at least a combination of a DNA ligase and a proof-reading DNA polymerase. For example, the DNA is incubated with 400 units Taq DNA ligase, 0.1 units E. coli DNA polymerase I, 20 units E. coli endonuclease IV, 5 units T4 PDG in 1× Thermopol buffer supplemented with 0.5 mM NAD⁺ and 100 μM dNTPs at room temperature for 15 minutes. Prior to the ligation step the blunted and repaired DNA can be incubated at 75° C. for 20 minutes to inactivate the E. coli DNA polymerase I.

The mix of a proof-reading DNA polymerase in the reaction mix is able to blunt the DNA ends for subsequent ligation to either primers or a plasmid.

### Example 17

### Enhanced Amplicon Yield from Fragmented DNA. Production of Larger DNA Pieces from Fragmented DNA for Downstream Processes Such as Amplification, DNA Sequencing, Microarray Analysis, and Hybridization Analysis

Fragmented DNA, from 0.1-1000 ng, is incubated with a recombination/DNA annealing proficient protein, such as E. coli RecA (NEB# M0249S, NEB, Ipswich, Mass.; West, S. C. Ann. Rev. Biochem. 61, 603-640 (1992)) and/or lambda beta protein (Rybalchenko, N., et al. Proc. Natl. Acad. Sci. USA, 101(49):17056-17060 (2004); Kmeic, E., & Holloman, W. K., J. Biol. Chem. 256(24):12636-12639 (1981)) in a standard reaction buffer and any required cofactors in a final volume of 5-1000 μL. An example of a standard reaction buffer is 10 mM Tris-HCl, pH 7.5 at 25° C., 2 mM MgCl₂, and 50 mM NaCl. When using RecA, 1 mM ATP is included in the standard reaction. Either simultaneous with or subsequent to the incubation with the RecA and/or beta protein the DNA is also contacted with a repair mix composed of at least a DNA ligase activity a DNA polymerase activity and any required cofactors, i.e., ATP, NAD⁺ and dNTPs. The repair mix contains 400 units Taq DNA ligase and 0.1 units E. coli pol I and, in addition, 5 units T4 PDG, 20 units endonuclease IV, 0.5 units E. coli UDG, 2.5 units endonuclease VIII, and/or 0.1 unit Fpg are added. Prior to incubation with the repair proteins the DNA fragments may be heat-denatured and the temperature reduced to less than 39° C. For example, the DNA in reaction buffer may be heated to 98° C. for 5 minutes then cooled down to less than 39° C. A standard reaction volume is 5 to 1000 μL and the incubation time is 1 to 60 minutes at 4-37° C. Typically, the RecA or beta protein is used at a 0.5:1 to 5:1 nucleotide to protein mole ratio.

Modifications to the above method include substituting RecA and/or beta protein with thermostable equivalents. Some examples of these proteins are ttRecA (Kato R, & Kuramitsu S., Eur J Biochem. 259(3):592-601 (1999)), Taq RecA, Tma RecA, and Apy RecA (Wetmur, J. G., et al. J Biol. Chem. 269(41):25928-35 (1994)). The use of thermostable proteins means that thermostable RecA or beta-like protein can be mixed with the DNA during the denaturation step. Any co-factors required for the protein activity are also included. In addition, repair enzymes as described above are added prior to or after denaturation. Note that for thermostable recombination proteins (RecA or beta-like protein) the proteins can be added to the reaction mixture for 1-60 minutes at 45° C.-75° C. to permit the optimal activity before the addition of non-thermostable repair proteins at temperatures of less than 39° C.

The repaired DNA can then be used in a subsequent process, for example PCR. For example, as a test system human genomic DNA is fragmented using sonication and size fractionated to give average fragment sizes clustered around 200 bp. 500 ng of the size-fractionated material is treated as described above. A titration of 5-100 ng of this repaired material is used in PCR reactions using primers that reliably generate 1, 2, and 4 kb amplicons from undamaged human genomic DNA. Examples of a primer set are

| | |
|---|---|
| DNMT-R: | |
| GGGGCACCTTCTCCAACTCATACT, | (SEQ ID NO: 34) |
| DNMT-1Fb: | |
| cctcatttggggaggggttatct, | (SEQ ID NO: 35) |
| DNMT-2Fc: | |
| cctgaaacaaggttgtggcatagc, | (SEQ ID NO: 36) |
| and | |
| DNMT-4Fb: | |
| gagtgagttgaaagtgctccataca. | (SEQ ID NO: 37) |

The same template titration is performed with the fragmented DNA. When the non-repaired and repaired DNA are compared, the repaired templates permit a visible amplicon on an agarose gel, visualized with UV light and ethidium bromide, to be generated with at least two fold lower amounts of template DNA.

The use of RecA and/or beta protein-like activities in conjunction with at least DNA ligase and DNA polymerase activities results in the detection of PCR amplicons at lower template amounts as compared to unrepaired DNA.

### Example 18

### Amplification of DNA from Stored Ancient Cave Bear Tissue Samples after Repair of DNA Damage

In contrast to modern material, the DNA extracted from ancient bones shows a variety of types of damage. The most common type of damage is fragmentation caused by single stand breaks, which lead to a reduced average molecule length of the extracted DNA, as well as non-enzymatic attacks such as irradiation and reactive oxygen species. (See: Hoss, et al. Nucleic Acids Res. 24(7):1304-7 (1996)). Repairing ancient DNA (aDNA) damage is important to improve the utility of the extracted DNA.

An ancient DNA was extracted as described in Pääbo Proc Natl Acad Sci USA 86(6):1939-43 (1989). Amplification of a 330 bp cave bear DNA (˜44,000 years old) was performed using primers CB F1 (CTATTTAAACTATTCCCTGGTACATAC) (SEQ ID NO:38) and CB R1 (GGAGCGAGAGGTACACGT) (SEQ ID NO:39) either with or without prior repair. The DNA repair mix was composed of 200 units/μL Taq DNA ligase, 0.1 units/μL E. coli PolI, 1 unit/μL T4 PDG, 15 units/μL endonuclease IV, 0.5 unit/μL E. coli UDG, 2.5 units/μL endonuclease VIII, and 0.1 unit/μL Fpg and stored in 20 mM Tris-HCl, pH 7.5 at 25° C., 100 mM NaCl, and 50% gycerol. To 2 of 4 thermocycler tubes each containing 2 μl aDNA, 1× Phusion™ DNA polymerase buffer, 100 μM dNTPs, and 0.5 mM NAD⁺ was added 1 μL of the repair enzyme mix. All were brought to a final volume of 45 μL with H₂O. The reaction solutions were incubated at room temperature for 15 minutes after which the primers (to 0.4 μM), dNTPs (to 100 μM), and 1 unit Phusion™ DNA polymerase were added to each. The solutions were placed into a Mycycler thermocycler (Bio-Rad, Hercules, Calif.) running the program: 98° C. for 30 s, one cycle; 98° C. for 10 sec, 58° C. for 20 s and 72° C. for 20 s, 30 cycles; 72° C. for 5 min, one cycle; then a 4° C. hold. The repaired PCR amplified DNA and control PCR amplified DNA (no repair) was used immediately in a second PCR amplification using nested primers. The amplification reaction with the nested primers used 1× Taq Master Mix (Catalog #M0270S, NEB, Ipswich, Mass.), 2 μL of the previous amplification, and primers CB F1 (CTATTTAAACTATTCCCTGGTACATAC) (SEQ ID NO:40) and CB F3 (GCCCCATGCATATAAGCATG) (SEQ ID NO:41) at a final concentration of 0.2 μM. The total reaction volume was 50 μL. The reaction was analyzed by applying 5 μl of each reaction to a 1% agarose gel, prepared, electrophoresed and visualized as described above.

The amount of mitochondrial DNA in the cave bear bone samples was estimated with the TaqMan® assay (Applied Biosystems, Foster City, Calif.) using primers 5′-AAAATGCCCTTTGGATCTTAAA-3′ (SEQ ID NO:43) and 5′-ACTGCTGTATCCCGTGGG-3′ (SEQ ID NO:44).

The amplified DNA is either used immediately in the DNA sequencing methodology or subjected to DNA purification and concentration. After purification the DNA is subjected to DNA sequencing (see Example 11).

Amplification from cave bear DNA using Phusion™ DNA polymerase and Taq DNA polymerase in nested PCR produced a detectable product of the desired amplicon size in one sample (CB3A). Treatment with the repair mix produced another amplicon from sample CB3B. Sequence analysis of amplicons from treated and untreated cave bear DNAs (CB3B sample) will reveal whether treatment with the repair mix significantly helped to remove PCR amplification errors associated with DNA modifications described in Hoss, et al. Nucleic Acids Res. 24(7):1304-7 (1996).

Treatment of the cave bear DNA template with the repair enzyme mix permitted Phusion™ DNA polymerase to more effectively produce the desired amplicon and to remove PCR amplification errors (FIG. 17).

### Example 19

### Repair of Various Types of DNA Damage by a Single Repair Mixture

Damage to DNA was induced by ultraviolet radiation, heat or acidic pH. A seven-enzyme mixture was found to effectively repair the damaged DNA.

### (a) Generating UV-Damaged DNA.

(i) Lambda DNA was damaged by exposure to UV as described in Example 6, with the exception that the UV incubation time was 5 minutes. The UV lamp output was 14.6 milliwatts/cm² The UV light intensity was measured using a UVX Digital Radiometer made by UVP, Inc., San Gabriel, Calif. The concentration of DNA was 50 ng/μL.

(ii) Human genomic DNA (Catalog #70572-3, Novagen, Madison, Wis.) was UV-damaged as described in Example 6 for 20 seconds. The concentration of DNA was 50 ng/μL.

### (b) Generating Acid pH and UV-Damaged Lambda DNA.

Acid and UV-damaged lambda DNA was generated by first treating the DNA in low pH as described in Example 2. The DNA was incubated for 120 minutes at 70° C. The damaged DNA was diluted to 50 ng/μL and exposed to UV light for 30s as described in Example 6.

### (c) Generating Acid pH Damaged Lambda DNA

(i) A preparation of lambda DNA (500 ng/ul) was treated as described in Example 2 to cause acid damage. The final concentration of acid damaged DNA was 232 ng/ul.

### (d) Generating Heat-Damaged Lambda DNA.

Lambda DNA was damaged by heat treatment as described in Example 1. The 180 second time point only was used in this example. The DNA concentration was 500 ng/μL.

### (e) Generating Oxidized Plasmid DNA.

Plasmid pWB407 was oxidized as described in Example 9. The amount of methylene blue in the reaction was 12 ng/μL (CHECK THIS). The DNA concentration was 50 ng/μL.

### DNA Repair

DNA which was damaged as described above was treated with a DNA repair mix prior to PCR.

The repair mix was formed from a cocktail of enzymes where 1 ul of the mixture contained:

200 units Taq DNA ligase
0.01 units-200 units Bst DNA polymerase*
0.01 units-5000 units E. coli endonuclease IV
0.01 units-200 units T4 PDG
0.001 units-1000 units E. coli UDG
0.001-1000 units E. coli endonuclease VIII
0.001-5 units E. coli Fpg
* Bst unit assay definition is described in the NEB catalog for the Bst DNA polymerase (full length). Also see Aliotta et al. (1996) Genet. Anal. 12:185-95.

The storage buffer for the repair enzyme cocktail was 20 mM Tris, pH 7.5, containing 100 mM NaCl and 50% glycerol. 1 μL of the repair cocktail was used per repair reaction in this Example. 100 μM dNTPs and 0.5 mM NAD⁺ were added to the cocktail.

The buffer used in the repair reaction was varied according to which DNA polymerase was selected for PCR. For example, ThermoPol buffer (NEB, Ipswich, Mass.) is preferably used for Taq DNA polymerase and therefore 1× Thermopol buffer was selected for the repair mix for UV-damaged lambda DNA, heat-damaged lambda DNA, acid and UV-damaged lambda DNA, and low pH damaged lambda DNA. GC buffer was preferably used for Phusion™ DNA polymerase and therefore 1× GC buffer (NEB, Ipswich, Mass.) was selected for the repair mix that involved methylene blue damaged pWB407 and UV-damaged human genomic DNA prior to amplification with Phusion™ DNA. After incubation at the above described temperatures and times the reactions were placed on ice.

UV irradiated lambda DNA and oxidized pWB407 were incubated with the DNA repair mix at either 37° C. for 5 minutes. Heat-damaged lambda DNA, UV-damaged human DNA, acid and UV-damaged lambda DNA, and low pH damaged lambda DNA were alternatively incubated at room temperature for 10-15 minutes. The reaction volume was 48.5 μL.

The amount of DNA in each repair reaction was as follows: 1 ng for UV-damaged lambda DNA, heat-damaged lambda DNA, and acid and UV-damaged lambda DNA; or

10 ng of oxidized lambda DNA; or
50 ng of UV-damaged human genomic DNA and low pH damaged lambda DNA.

The negative control reaction in which no repair occurred was treated as above except that the repair enzymes were not added. However, the appropriate volume of enzyme storage buffer was used.

For all amplification reactions described below, thermocycling was carried out as follows:

A Bio-Rad Mycycler was used that ran the following program: 2 min at 95° C. for 1 cycle, then 10 sec at 95° C., 30 sec at 65° C., and 1 min at 72° C. for 25 cycles, and finally 5 min at 72° C. for 1 cycle and a 4° C. hold (Bio-Rad, Hercules, Calif.).

The product of amplification was visualized on a 1% agarose gel stained with ethidium bromide (see FIG. 19).

DNA amplification reactions

(a) Amplification from the UV-damaged lambda DNA. DNA amplification was performed using primers L30350F (SEQ ID NO:2) and GATGACGCATCCTCACGATAATATCCGG (L71-1R) (SEQ ID NO:47) according to the method of Wang et al. Nucl. Acids Res. 32:1197-1207 (2004).

Primers (final concentration 0.4 uM) were added to the repair mix containing UV-damaged lambda DNA or a negative control. 100 μM dNTPs (final concentration 200 μM) and 2.5 units of Taq DNA polymerase were also added. The volume of the reaction mix was 50 μL.

A Bio-Rad Mycycler was used that ran the following program: 2 min at 95° C. for 1 cycle, then 10 sec at 95° C., 30 sec at 65° C., and 1 min at 72° C. for 25 cycles, and finally 5 min at 72° C. for 1 cycle and a 4° C. hold (Bio-Rad, Hercules, Calif.).

(b) Amplification from Heat-Damaged Lambda DNA.

DNA amplification was performed using primers L30350F (SEQ ID NO:2) and L72-2R (SEQ ID NO:8) according to the method of Wang et al. Nucl. Acids Res. 32:1197-1207 (2004).

Primers (final concentration 0.4 uM) were added to the repair mix containing heat-damaged lambda DNA or a negative control. 100 μM dNTPs (final concentration 200 uM) and 2.5 units of Taq DNA polymerase were also added. The volume of the reaction mix was 50 μL.

The reactions were transferred to a Bio-Rad Mycycler that ran the following program: 2 min at 95° C. for 1 cycle, then 10 sec at 95° C., 30 sec at 65° C., and 2 min at 72° C. for 25 cycles, and finally a 4° C. hold (Bio-Rad, Hercules, Calif.).

(c) Amplification from Oxidized pWB407 Plasmid DNA

DNA amplification was performed using primers 316-138 (SEQ ID NO:9) and 316-137 (SEQ ID NO:10).

Primers (final concentration 0.4 uM) were added to the repair mix containing oxidized pWB407 DNA or a negative control. 100 μM dNTPs (final concentration 200 uM) and 1 unit of Phusion™ DNA polymerase were also added. The volume of the reaction mix was 50 μL.

The reactions were transferred to a Bio-Rad Mycycler that ran the following program: 30 sec at 98° C. for 1 cycle, then 10 sec at 98° C., 20 sec at 68° C., and 1 min 15 sec at 72° C. for 30 cycles, and finally 5 min at 72° C. for 1 cycle and a 4° C. hold (Bio-Rad, Hercules, Calif.).

(d) Amplification from UV-Damaged Human Genomic DNA

DNA amplification was performed using primers DNMT-4Fb (SEQ ID NO:37) and DNMT-R (SEQ ID NO:34).

Primers (final concentration 0.4 μM) were added to the repair mix containing UV-damaged human DNA or a negative control. 100 μM dNTPs (final concentration 200 uM) and 2.5 units of Taq DNA polymerase were also added. The volume of the reaction mix was 50 μL.

The reactions were transferred to a Bio-Rad Mycycler that ran the following program: 30 sec at 98° C. for 1 cycle, then 10 sec at 98° C., 30 sec at 68.5° C., and 2 min at 72° C. for 30 cycles, and finally 5 min at 72° C. for 1 cycle and a 4° C. hold (Bio-Rad, Hercules, Calif.).

(e) Amplification from Acid and UV-Damaged Lambda DNA.

DNA amplification was performed using primers L30350F (SEQ ID NO:2) and L72-5R (SEQ ID NO:1) according to the method of Wang et al. Nucl. Acids Res. 32:1197-1207 (2004).

Primers (final concentration 0.4 μM) were added to the repair mix containing acid and UV-damaged lambda DNA or a negative control. 100 μM dNTPs (final concentration 200 μM), 2.5 units of Taq DNA polymerase and 0.05 units of Vent® DNA polymerases were also added. The volume of the reaction mix was 50 μL.

The reactions were transferred to a Bio-Rad Mycycler that ran the following program: 2 min at 95° C. for 1 cycle, then 30 sec at 94° C., 30 sec at 63° C., and 5 min at 72° C. for 25 cycles, and finally 5 min at 72° C. for 1 cycle and a 4° C. hold (Bio-Rad, Hercules, Calif.).

(f) Amplification from Low pH Damaged Lambda DNA

DNA amplification was performed using primers L30350F (SEQ ID NO:2) and L71-10R (SEQ ID NO:5) according to the method of Wang et al. Nucl. Acids Res. 32:1197-1207 (2004).

To the low-pH-damaged lambda DNA repair reaction and negative control was added the primers to 0.4 μM of each, 100 μM dNTPs, and 2.5 units of Taq and 0.05 units of Vent® DNA polymerases. The final PCR volume was 50 μL.

The reactions were transferred to a Bio-Rad Mycycler that ran the following program: 20 sec at 95° C. for 1 cycle, then 5 sec at 95° C. and 10 min at 72° C. for 30 cycles, and finally 5 min at 72° C. for 1 cycle and a 4° C. hold (Bio-Rad, Hercules, Calif.).

### Example 20

### Detrimental Effect of ATP on PCR Amplification

Primers L30350F (SEQ ID NO:2) and L72-5R (SEQ ID NO:1) were used in PCR to amplify a 5 kb amplicon from lambda phage DNA in the presence of differing concentrations of adenosine triphosphate (ATP, Sigma Chemical Company, St. Louis, Mo., catalog #A-2383). The 50 μL PCR reactions contained 50 picograms lambda DNA, 1× HF buffer (NEB #F-518, NEB, Ipswich, Mass.), 200 μM dNTPs (NEB #F560PL, NEB, Ipswich, Mass.), 0.5 μM primer L30350F, 0.5 μM L72-5R, 1 unit Phusion™ DNA polymerase (NEB#F530PL, NEB, Ipswich, Mass.), ATP to the concentration indicated in the lanes in FIG. 20, and H₂O to bring the volume to 50 μL.

Thermocycling was performed using a Bio-Rad Mycycler that ran the following program: 30 sec at 98° C. for 1 cycle, then 5 sec at 98° C., 1 min 15 sec at 72° C. for 25 cycles, and finally 5 min at 72° C. for 1 cycle and a 4° C. hold (Bio-Rad, Hercules, Calif.).

The product of amplification was visualized on a 1% agarose gel stained with ethidium bromide (see FIG. 20). The broad range molecular weight marker (NEB#N3200S, NEB, Ipswich, Mass.) was applied to the left most lane in each gel.

### Example 21

### Determining an Effective Amount of AP Endonuclease Activity

The effective concentration of AP endonuclease for use in a repair reaction is a concentration that results in specific endonuclease activity at AP sites while avoiding non-specific degradation resulting from exonuclease activity. A range of effective concentrations has been determined using the following experimental protocol.

A synthetic oligonucleotide with a uracil base inserted near the middle of the sequence was base-paired with a complementary DNA to generate a double-stranded template. The uracil base was excised by UDG in the reaction to create an AP site that could then be acted upon by the AP endonuclease to be tested. The oligonucleotide with the uracil group was labeled at both the 5′ and 3′ ends so that it's size could be monitored by gel electrophoresis. An effective AP endonuclease concentration is one that detectably cleaves the oligonucleotide at the generated AP site, but that does not detectably degrade the oligonucleotide non-specifically.

A typical reaction used 1, for example, oligonucleotide 287: GATTTCATTTTTATTUATAACTTTACTTATATTGT (SEQ ID NO:45) and oligonucleotide 288: CAATATAAGTAAAGTTATAAATAAAAATGAAATC (SEQ ID NO:46). Oligonucleotide 287 was labeled at both the 5′ and 3′ ends. The oligonucleotides were annealed to form double-stranded DNA. The test reactions contained 0.5 pmol/μL annealed DNA substrate, 0.05 units/μL UDG, and 1× test buffer. The test buffer could be, for example, NEBuffer 3 (NEB, Ipswich, Mass.). 1× NEBuffer 3 was composed of 100 mM NaCl, 50 mM Tris-HCl, 10 mM MgCl₂, 1 mM DTT, pH 7.9 at 25° C. (NEB, Ipswich, Mass.). A serial dilution of AP endonuclease activity was made in these reaction conditions. The final reaction volume was made 10 μL with H₂O. The reactions were incubated at a chosen temperature, typically the temperature of optimal activity of the AP endonuclease, for 1 hour. The reactions were stopped by adding stop dye to 1×. A typical 5× stop dye was composed of 12% ficoll, 0.01% bromphenol blue, 0.02% xylene cyanol, 7M Urea, 50% formamide, 1% SDS, 89 mM Tris,

2 mM EDTA, and 89 mM borate, pH 8.3. The reactions were analyzed by denaturing gel electrophoresis.

1. A method for repairing a damaged polynucleotide so as to enhance at least one of fidelity and yield of a copied or amplified product of the polynucleotide, comprising:
(a) incubating the polynucleotide in a reaction mixture comprising: an effective amount of at least one apurinic/apyrimidinic (AP) endonuclease; a DNA ligase; and at least one of NAD⁺ or ATP as a cofactor; and
(b) enhancing at least one of fidelity and yield of the copied or amplified product.

2. A method according to claim 1, wherein the polynucleotide is DNA.

3. A method according to claim 1, wherein the ligase is an NAD⁺-dependent ligase and the reaction mixture contains NAD⁺.

4. A method according to claim 3, wherein the ligase is Taq DNA ligase or E. coli DNA ligase.

5. A method according to claim 1, wherein step (a) further comprises: amplifying the polynucleotide in the reaction mixture.

6. A method according to claim 1, wherein the polynucleotide in step (a) is amplified without denaturing or removing the at least one AP endonuclease in the reaction mixture.

7. A method according to claim 6, wherein amplification occurs by means selected from the group consisting of: PCR amplification, helicase-dependent amplification, strand-displacement amplification, rolling circle amplification and whole genome amplification.

8. A method according to claim 5, wherein the polynucleotide for amplification is in a size range of 50 nucleotides to 100,000 nucleotides.

9. A method according to claim 1, wherein the polynucleotide is obtained from a source selected from the group consisting of: a natural source, preserved biological material, forensic evidence, ancient material of biological origin, a tissue biopsy and chemical synthesis.

10. A method according to claim 1, wherein the damaged polynucleotide is characterized by one or more types of damage selected from AP sites, mutagenized nucleotides, modified nucleotides, nicks, gaps, DNA-DNA or DNA-protein cross-links, fragmentation and DNA-RNA crosslinks.

11. A method according to claim 1, wherein the at least one AP endonuclease comprises: an endonuclease obtainable from a bacterium, a mammal, an archaea or a virus.

12. A method according to claim 1, wherein the at least one AP endonuclease comprises an endonuclease obtainable from E. coli, human cells or Thermococcus species.

13. A method according to claim 1, wherein the reaction mixture further comprises: a DNA polymerase.

14. A method according to claim 13, wherein the DNA polymerase is a Family A polymerase.

15. A method according to claim 13, wherein the DNA polymerase is a Family B polymerase.

16. A method according to claim 13, wherein the DNA polymerase is a member of the Y family of DNA polymerases.

17. A method according to claim 13, wherein the DNA polymerase is selected from the group consisting of: a Taq DNA polymerase, an E. coli DNA polymerase, a Bst DNA polymerase, and a phage T4 DNA polymerase.

18. A method according to claim 13, wherein the DNA polymerase is selected from E. coli pol IV, E. coli pol V, human pol kappa, human pol eta, Sso Dpo4, Sac Dbh, Sce pol zeta, a phage T7 DNA polymerase and human pol iota.

19. A method according to claim 1, wherein the reaction mixture further comprises: a T7 endonuclease I or mutant thereof.

20. A method according to claim 1 or 13, wherein the reaction mixture further comprises: T4 pyrimidine dimer glycosylase (PDG).

21. A method according to claim 1 or 13, wherein the reaction mixture further comprises: formamidopyrimidine [fapy]-DNA glycosylase (Fpg).

22. A method according to claim 1 or 13, wherein the reaction mixture further comprises: at least one of UvrA, UvrB and UvrC and optionally UvrD or Cho.

23. A method according to claim 1 or 13, wherein the reaction mixture further comprises: at least one of endonuclease VIII, endonuclease V or endonuclease III.

24. A method according to claim 1 or 13, wherein the reaction mixture further comprises: at least one of uracil DNA glycosylase (UDG) or alkyl adenine DNA glycosylase (Aag).

25. A method according to claim 1, wherein incubating the polynucleotide in the reaction mixture is accomplished at a substantially single temperature so as to enhance at least one of yield or fidelity.

26. A kit, comprising: two or more enzymes for forming a reaction mixture wherein at least one of the enzymes is a DNA ligase, and at least one of the enzymes is an AP endonuclease at a concentration in the range of 0.0001 units/μl to 100 units/μl in the reaction mixture, the two or more enzymes being formulated for addition to a damaged polynucleotide preparation to enhance repair of the polynucleotide; and instructions for its use.

27. A polynucleotide repair mixture, comprising: an effective amount of at least one AP endonuclease, a DNA ligase, and a DNA polymerase, wherein the repair mixture can be added to a polynucleotide and the polynucleotide can be amplified without removing or degrading the polynucleotide repair mixture; and wherein the repair mixture enhances at least one of yield and fidelity of a copied polynucleotide.

28. A polynucleotide repair mixture according to claim 27, wherein the DNA ligase is an E. coli DNA ligase or a Taq DNA ligase.

29. A polynucleotide repair mixture according to claim 27, wherein the AP endonuclease is E. coli endonuclease IV.

30. A polynucleotide repair mixture according to claim 27, wherein the DNA polymerase is Bst DNA polymerase.

31. A polynucleotide repair mixture according to claim 27, further comprising: T4 PDG.

32. A polynucleotide repair mixture according to claim 27, further comprising: E. coli Fpg.

33. A polynucleotide repair mixture according to claim 27, further comprising: at least one of UvrA, UvrB, UvrC and optionally UvrD or Cho.

34. A polynucleotide repair mixture according to claim 27, further comprising: at least one of endonuclease VIII, endonuclease V or endonuclease III.

35. A polynucleotide repair mixture according to claim 27, further comprising: at least one of UDG and Aag.

36. A polynucleotide repair mixture according to claim 27, further comprising: a PDG, a UDG, an endonuclease VIII and an Fpg.

37. A polynucleotide repair mixture according to claim 36, wherein one or more of the DNA ligase, DNA polymerase, AP endonuclease, PDG, UDG, endonuclease VIII and Fpg is obtained from E. coli.

38. A polynucleotide repair mixture according to claim 37, wherein the AP endonuclease, endonuclease VIII, UDG, and Fpg are obtained from E. coli.

39. A polynucleotide repair mixture according to claim 37, wherein the PDG is T4 PDG, the DNA ligase is Taq DNA ligase, and the polymerase is Bst DNA polymerase.

40. A polynucleotide repair mixture according to claim 39, wherein the effective concentration of T4 PDG is in the range of 0.0001 units/μl to 4 units/μl.

41. A polynucleotide repair mixture according to claim 39, wherein the effective concentration of Taq DNA ligase is in the range of 0.00001 units/μl to 100 units/μl.

42. A polynucleotide repair mixture according to claim 39, wherein the effective concentration of Bst DNA polymerase is in the range of 0.00001 units/μl to 2 units/μl.

43. A polynucleotide repair mixture according to claim 29, wherein the effective concentration of E. coli endonuclease IV is in the range of 0.0001 units/μl to 100 units/μl.

44. A polynucleotide repair mixture according to claim 34, wherein the effective concentration of endonuclease VIII is in the range of 0.00001 units/μl to 20 units/μl.

45. A polynucleotide repair mixture according to claim 35, wherein the effective concentration of UDG is in the range of 0.00001 units/μl to 20 units/μl.

46. A polynucleotide repair mixture according to claim 32, wherein the effective concentration of Fpg is in the range of 0.000001 units/μl to 0.1 units/μl.

47. A polynucleotide repair mixture, comprising: Bst DNA polymerase, AP endonuclease, endonuclease VIII, a DNA ligase, Fpg, PDG and UDG.

48. A polynucleotide repair mixture according to claim 47, wherein the Bst polymerase has a concentration in the range of 0.00001 units/μl to 2 units/μl, the AP endonuclease has a concentration in the range of 0.0001 units/μl to 100 units/μl, the endonuclease VIII has a concentration in the range of 0.00001 units/μl to 20 units/μl, the ligase has a concentration in the range of 0.00001 units/μl to 100 units/μl, the Fpg has a concentration in the range of 0.000001 units/μl to 0.1 units/μl, the PDG has a concentration in the range of 0.0001 units/μl to 4 units/μl and UDG has a concentration in the range of 0.00001 units/μl to 20 units/μl.

49. A method for cloning or sequencing a polynucleotide fragment, comprising:
repairing sequence errors in the polynucleotide fragment by means of a polynucleotide repair mixture according to claim 27; and
cloning or sequencing the polynucleotide fragment.

50. A method according to claim 49, wherein the repair mixture is capable of blunt-ending the polynucleotide fragment for cloning into a vector.

51. A method for enhancing the yield of a copied or amplified polynucleotide, comprising:
(a) obtaining at least a first pair and a second pair of primers wherein the second pair of primers is nested within the first set of primers when hybridized to the polynucleotide;
(b) subjecting the polynucleotide to a polynucleotide repair mixture according to claim 27;
(c) amplifying the polynucleotide with the first set of primers;
(d) amplifying the product of (c) with the second set of primers; and
(e) obtaining an enhanced yield of amplified polynucleotide.

52. A method according to claim 49 or 51, wherein the polynucleotide repair mixture further comprises: a PDG, an endonuclease VIII, Fpg, and optionally UDG and wherein the AP endonuclease is an endonuclease IV.

53. A method according to claim 52, wherein the ligase is Taq DNA ligase, and the PDG is T4 PDG, and the DNA polymerase, the endonuclease IV, endonuclease VIII, Fpg and optionally UDG are obtained from E. coli.

54. A method for sequencing a polynucleotide, comprising:
(a) contacting the polynucleotide with the polynucleotide repair mixture of claim 27; and
(b) sequencing the polynucleotide.

55. A method for copying or amplifying a fragmented DNA, comprising:
(a) contacting the fragmented DNA with the repair mixture of claim 27;
(b) optionally adding a recombination-competent protein; and
(c) amplifying or copying the fragmented DNA.

56. A method according to claim 55, wherein the recombination-competent protein is an E. coli RecA or phage lambda beta protein.


SEQUENCE LISTING (47 sequences)

SEQ ID NO: 1 - DNA, 23 bp, artificial
  primer
cgaacgtcgcgcagagaaacagg

SEQ ID NO: 2 - DNA, 20 bp, artificial
  primer
cctgctctgccgcttcacgc

SEQ ID NO: 3 - DNA, 25 bp, artificial
  primer
tttttagcaatacactacacagcaa

SEQ ID NO: 4 - DNA, 19 bp, artificial
  primer
attacgccaatcgatcacg

SEQ ID NO: 5 - DNA, 27 bp, artificial
  primer
gcacagaagctattatgcgtccccagg

SEQ ID NO: 6 - DNA, 25 bp, artificial
  primer
ggggggagagtttgatcmtggctca

SEQ ID NO: 7 - DNA, 28 bp, artificial
  primer
ggggggtacggytaccttgttacgactt

SEQ ID NO: 8 - DNA, 24 bp, artificial
  primer
ccatgattcagtgtgcccgtctgg

SEQ ID NO: 9 - DNA, 29 bp, artificial
  primer
tgtcgatcaggatgatctggacgaagagc

SEQ ID NO: 10 - DNA, 33 bp, artificial
  primer
cgaaagctttcaaggatcttaccgctgttgaga

SEQ ID NO: 11 - DNA, 813 bp, unknown
  Thermus thermophilus endonuclease IV
atgccgcgctacgggttccacctttccatcgccgggaaaaagggcgtggccggggcggtggaggaggccaccgccctcggcctcaccgctttccagatcttcgccaaaagcccgcggagctggcgcccaagggccctctccccggccgaggtggaggccttccgcgccttaagggaggcctccgggggcctccccgccgtgatccacgcctcctacctggtcaacctgggggcggagggggagctttgggagaagagcgtggcgagcctggcggacgacctggagaaggccgccctcctcggggtggagtacgtggtcgtccaccccggctcgggccgccccgagcgggtcaaggaaggggccctcaaggccctgcgcctcgccggcgtccgctcccgccccgtcctcctcgtggagaacaccgccgggggcggggagaaggtgggggcgcggtttgaggagctcgcctggctcgtggcggacacccccctccaggtctgcctggacacctgccacgcctacgccgccgggtacgacgtggccgaggaccccttgggggtcctggacgccctggaccgggccgtgggcctggagcgggtgcccgtggtccacctcaacgactccgtgggcggcctcggaagccgcgtggaccaccacgcccacctcctccagggaaagatcggggaggggctcaagcgcgtcttcttggacccgaggctcaaggaccgggtcttcatcctggaaacccccaggggaccggaggaggacgcctggaacctccgggtcctcagggcctggctcgaggaggcctaa

SEQ ID NO: 12 - DNA, 24 bp, artificial
  primer
cgccagggttttcccagtcacgac

SEQ ID NO: 13 - DNA, 24 bp, artificial
  primer
agcggataacaatttcacacagga

SEQ ID NO: 14 - DNA, 45 bp, unknown
  synthetic oligo
ggcggcctcgaggcgaaacgccgcaactgctttccgggcgatacc

SEQ ID NO: 15 - DNA, 45 bp, unknown
  synthetic oligo
cggcacgccatcaatctgcaccagaatgcgggtatcgcccggaaa

SEQ ID NO: 16 - DNA, 45 bp, unknown
  synthetic oligo
attgatggcgtgccgcagaaaattaccctgcgcgaactgtatgaa

SEQ ID NO: 17 - DNA, 45 bp, unknown
  synthetic oligo
catgttttcatagcgttcatcttcaaacagttcatacagttcgcg

SEQ ID NO: 18 - DNA, 45 bp, unknown
  synthetic oligo
cgctatgaaaacatggtgtatgtgcgcaaaaaaccgaaacgcgaa

SEQ ID NO: 19 - DNA, 45 bp, unknown
  synthetic oligo
ggtttccaggtcaatgctatacactttaatttcgcgtttcggttt

SEQ ID NO: 20 - DNA, 46 bp, unknown
  synthetic oligo
attgacctggaaaccggcaaagtggtgctgaccgatattagaagat

SEQ ID NO: 21 - DNA, 45 bp, unknown
  synthetic oligo
cagatgatcggtcgccggcgctttaatcacatcttcaatatcggt

SEQ ID NO: 22 - DNA, 45 bp, unknown
  synthetic oligo
gcgaccgatcatctgattcgctttgaactggaagatggccgcagc

SEQ ID NO: 23 - DNA, 45 bp, unknown
  synthetic oligo
cagcaccggatgatccacggtggtttcaaagctgcggccatcttc

SEQ ID NO: 24 - DNA, 45 bp, unknown
  synthetic
gatcatccggtgctggtgtatgaaaacggccgctttattgaaaaa

SEQ ID NO: 25 - DNA, 45 bp, unknown
  synthetic oligo
tttatcgccttctttcacttcaaacgcgcgtttttcaataaagcg

SEQ ID NO: 26 - DNA, 45 bp, unknown
  synthetic oligo
aaagaaggcgataaagtgctggtgagcgaactggaactggtggaa

SEQ ID NO: 27 - DNA, 45 bp, unknown
  synthetic oligo
tttcgggttatcctggctgctgctgctctgttccaccagttccag

SEQ ID NO: 28 - DNA, 45 bp, unknown
  synthetic oligo
caggataacccgaaaaacgaaaacctgggcagcccggaacatgat

SEQ ID NO: 29 - DNA, 45 bp, unknown
  synthetic oligo
atatttaatgtttttaatttccagcagctgatcatgttccgggct

SEQ ID NO: 30 - DNA, 48 bp, unknown
  synthetic oligo
aaaaacattaaatatgtgcgcgcgaacgatgattttgtgtttagcctg

SEQ ID NO: 31 - DNA, 47 bp, unknown
  synthetic oligo
ttaataatcacgttatgatattttttcgcgttcaggctaaacacaaa

SEQ ID NO: 32 - DNA, 44 bp, unknown
  synthetic oligo
taacgtgattattaacgaaaacattgtgacccatgcgtgcgatg

SEQ ID NO: 33 - DNA, 47 bp, unknown
  synthetic oligo
gccgccctgcagaccggtcagatcttcatcgccatcgcacgcatggg

SEQ ID NO: 34 - DNA, 24 bp, artificial
  primer
ggggcaccttctccaactcatact

SEQ ID NO: 35 - DNA, 23 bp, artificial
  primer
cctcatttggggaggggttatct

SEQ ID NO: 36 - DNA, 24 bp, artificial
  primer
cctgaaacaaggttgtggcatagc

SEQ ID NO: 37 - DNA, 25 bp, artificial
  primer
gagtgagttgaaagtgctccataca

SEQ ID NO: 38 - DNA, 27 bp, artificial
  primer
ctatttaaactattccctggtacatac

SEQ ID NO: 39 - DNA, 18 bp, artificial
  primer
ggagcgagaggtacacgt

SEQ ID NO: 40 - DNA, 27 bp, artificial
  primer
ctatttaaactattccctggtacatac

SEQ ID NO: 41 - DNA, 20 bp, artificial
  primer
gccccatgcatataagcatg

SEQ ID NO: 42 - DNA, 3474 bp, unknown
  plasmid pNEB0.92U
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttcctgcagggtttaaacgctgaggagacatatggccgccgagtctcagttaaaacgtgtgatcgaaacgctgcgccgtctgggtattgaagaggtgctgaaactggaacgtcgtgatcctcagtatcgcgctgtttgcaatgtggtcaagcgtcatggcgaaactgtgggcagccgtttagctatgttaaacgccctgatttcatatcgcctgaccggtaagggtgaggagcattgggaatatttcggcaaatatttcagtcagttagaagtgattgatctgtgccgtgatttcttaaaatatattgagaccagcccgttcctgaaaatcggtgtcgaggcgcgcaagaaacgcgcgttaaaggcctgcgactacgtccctaacttggaagacttgggcctgaccctgcgtcaattaagccacatcgttggtgcacgccgtgagcagaagacgttggtcttcacaatcaagatcctgaactatgcatatatgtgcagccgcggtgttatcgcgtgttgccgttcgatattccaattcctgtggattaccgtgttgcacgcttgacctggtgcgccggtctgatcgatttcccgccggaggaggccttgcgccgctacgaggctgtgcagaaaatctgggatgccgtggcgcgcgaaactggtattcctccattgcacttggacaccctgttatggttggccggtcgcgcggtgctgtatggtgaaaacctgcatggtgtgccgaaagaggtcatcgctctgttccaatggcgcggcggctgccgtccgcctagcgagtaaaccccctcagcttaattaaggcgcgcctgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 43 - DNA, 22 bp, artificial
  primer
aaaatgccctttggatcttaaa

SEQ ID NO: 44 - DNA, 18 bp, artificial
  primer
actgctgtatcccgtggg

SEQ ID NO: 45 - DNA, 35 bp, unknown
  primer
gatttcatttttattuataactttacttatattgt

SEQ ID NO: 46 - DNA, 34 bp, unknown
  primer
caatataagtaaagttataaataaaaatgaaatc

SEQ ID NO: 47 - DNA, 28 bp, unknown
  primer
gatgacgcatcctcacgataatatccgg